(19) United States
(12) Patent Application Publication
(10) Pub. No.: US 2007/0191393 A1
(43) Pub. Date: Aug. 16, 2007

(54) Macrocyclic anilinopyrimidines with substituted sulphoximine as selective inhibitors of cell cycle kinases

(76) Inventors: Ulrich Lucking, Berlin (DE); Gerhard Siemeister, Berlin (DE); Benjamin Bader, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/648,891

(22) Filed: Jan. 3, 2007

Related U.S. Application Data
(60) Provisional application No. 60/835,862, filed on Aug. 7, 2006.

(30) Foreign Application Priority Data
Jan. 3, 2006 (EP) 06090001.6

(51) Int. Cl. A61K 31/519; C07D 487/04

(52) U.S. Cl. 514/257; 540/472

(57) ABSTRACT

The invention relates to macrocyclic anilinopyrimidines with substituted sulphoximine of the general formula I, processes for their preparation, and their use as medicaments.

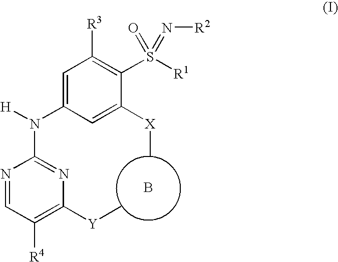

This application claims the benefit of the filing date of U.S. Provisional Application Ser. No. 60/835,862 filed Jan. 3, 2006.

The invention relates to macrocyclic anilinopyrimidines with substituted sulphoximine, processes for their preparation, and their use as medicaments.

Many biological processes such as, for example, DNA replication, energy metabolism, cell growth or cell differentiation in eukaryotic cells are regulated by reversible phosphorylation of proteins. The degree of phosphorylation of a protein has an influence inter alia on the function, localization or stability of proteins. The enzyme families of protein kinases and protein phosphatases are responsible respectively for the phosphorylation and dephosphorylation of proteins.

The sequential activity of the cell cycle kinases from the families of cyclin-dependent kinases (CDK), of polo-like kinases (Plk) and of Aurora kinases controls the division and thus the replication of a cell. These cell cycle kinases are therefore particularly interesting targets for the development of small inhibitory molecules which can be used for the treatment of cancer or other disorders which are caused by disturbances of cell proliferation.

Cell cycle kinases can be differentiated in terms of the phase of the cell cycle regulated by them:

a) Type 1 Cell Cycle Kinases

Type 1 cell cycle kinases mean in the context of the present invention all cell cycle kinases whose activity is not restricted to mitosis.

Type 1 cell cycle kinases include substantially the cyclin-dependent kinases (cdk) and the polo-like kinases (Plk).

b) Type 2 Cell Cycle Kinases

Type 2 cell cycle kinases mean in the context of the present invention all cell cycle kinases whose activity in the cell cycle is restricted to the M phase (mitosis).

The type 2 cell cycle kinases include substantially the Aurora kinases.

Inhibition of type 1 cell cycle kinases, such as CDK or Plk, precludes hitting the tumour cell in the more sensitive M phase because it is already arrested in an earlier phase of the cell cycle.

There is thus a need for compounds which are selective against type 1 cell cycle kinases, such as CDK, and inhibit type 2 cell cycle kinases.

There is furthermore a need for structures which, besides the selectivity against type 1 cell cycle kinases and inhibition of type 2 cell cycle kinases, inhibit tumour growth through inhibition of one or more further kinases (multi-target tumour growth inhibitors=MTGI).

Additional inhibition of the following kinase families is preferred:
receptor tyrosine kinases which regulate angiogenesis (angiogenic receptor tyrosine kinases), such as, for example, the receptor tyrosine kinases which are involved in the vascular endothelial growth factor (VEGF)/VEGF receptor system, fibroblast growth factor (FGF)/FGF receptor system, in the Eph ligand/EphB4 system, and in the Tie ligand/Tie system,
receptor tyrosine kinases whose activity contributes to the proliferation of cells (proliferative receptor tyrosine kinases), such as, for example, receptor tyrosine kinases which are involved in the platelet-derived growth factor (PDGF) ligand/PDGF receptor system, c-kit ligand/c-kit receptor system and in the FMS-like tyrosine kinase 3 (Flt-3) ligand/Flt-3 system,
checkpoint kinases which monitor the ordered progression of cell division, such as, for example, ATM and ATR, Chk1 and Chk2, Mps1, Bub1 and BubR1,
kinases whose activity protects the cell from apoptosis (anti-apoptotic kinases, kinases in so-called survival pathways), such as, for example, Akt/PKB, PDK1, IkappaB kinase (IKK), Pim1, and integrin-linked kinase (ILK),
kinases which are necessary for the migration of tumour cells (migratory kinases), such as, for example, focal adhesion kinase (FAK) and Rho kinase (ROCK).

The structures of the following patent applications form the structurally close prior art:

WO 2002/096888 discloses anilinopyrimidine derivatives as inhibitors of cyclin-dependent kinases. A sulphoximine substitutent is not disclosed for the aniline.

WO 2004/026881 discloses macrocyclic anilinopyrimidine derivatives as inhibitors of cyclin-dependent kinases. A possible sulphoximine substitutent for the aniline is disclosed only unsubstituted on the nitrogen atom of the sulphoximine.

WO 2005/037800 discloses open anilinopyrimidine derivatives as inhibitors of cyclin-dependent kinases. A sulphoximine substitutent is not disclosed for the aniline.

It is common to all these structures of the prior art that they inhibit type 1 cell cycle kinases, i.e. are not selective against type 2 cell cycle kinases.

Starting from this prior art, it is the object of the present invention to provide inhibitors of the cell cycle having specifically selected kinase selectivities.

There is a particular need for compounds which are selective against type 1 cell cycle kinases, such as cyclin-dependent kinases, and simultaneously inhibit type 2 cell cycle kinases, such as Aurora.

The object of the present application is achieved by compounds of the general formula I which have a sulphoximine substitutent substituted on the nitrogen.

It has surprisingly been found that substitution of the sulphoximine nitrogen leads to compounds which selectively inhibit type 2 cell cycle kinases, in particular inhibit Aurora and, at the same time, are selective against type 1 cell cycle kinases, such as cyclin-dependent kinases.

Substitution of the sulphoximine nitrogen atom moreover opens up the possibility of providing compounds which, besides inhibiting type 2 cell cycle kinases, inhibit a further kinase, so that tumour growth is efficiently inhibited, in particular a kinase from the kinase families of the receptor tyrosine kinases, of checkpoint kinases, of anti-apoptotic kinases or of migratory kinases.

The object of the present invention is achieved by compounds of the general formula I 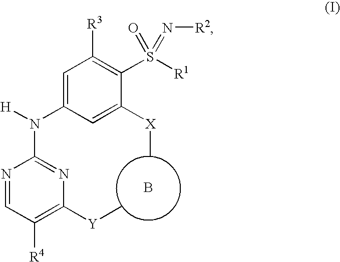
in which
B is a prop-1,3-ylene, but-1,4-ylene, pent-1,5-ylene or hex-1,6-ylene group which may be substituted one or more times, identically or differently, by
   (i) hydroxy, —NR¹¹R¹², cyano, halogen, —CF₃, —C₁-C₆-alkoxy, —NR¹³—C(O)—C₁-C₃-alkyl, —NR¹³—SO₂—C₁-C₃-alkyl, —OCF₃ and/or
   (ii) a C₁-C₆-alkyl radical which is optionally substituted one or more times, identically or differently, by hydroxy, —NR¹¹R¹², cyano, halogen, —CF₃, —C₁-C₆-alkoxy, —NR¹³—C(O)—C₁-C₃-alkyl, —NR¹³—SO₂—C₁-C₃-alkyl or —OCF₃,
R¹ is
   (i) a C₁-C₆-alkyl radical which is optionally substituted one or more times, identically or differently, by hydroxy, —NR¹¹R¹², cyano, halogen, C₁-C₆-alkoxy, —CF₃ and/or —OCF₃, or
   (ii) a C₃-C₇-cycloalkyl ring which is optionally substituted one or more times, identically or differently, by hydroxy, —NR¹¹R¹², cyano, halogen, —CF₃, C₁-C₆-alkoxy, —OCF₃ and/or C₁-C₆-alkyl, or
   (iii) a C₆-aryl ring which is optionally substituted one or more times, identically or differently, by hydroxy, —NR¹¹R¹², cyano, halogen, —CF₃, C₁-C₆-alkoxy, —OCF₃ and/or C₁-C₆-alkyl, or
   (iv) a heteroaryl ring which is optionally substituted one or more times, identically or differently, by hydroxy, —NR¹¹R¹², cyano, halogen, —CF₃, C₁-C₆-alkoxy, —OCF₃ and/or C₁-C₆-alkyl and has 5 or 6 ring atoms,
R² is R⁵, —SO₂—R⁶, —C(O)O—R⁶, —C(O)—R⁶, —C(O)—NR¹¹R¹², —C(S)—NR¹¹R¹², —Si(R⁷R⁸R⁹), —R¹⁰—Si(R⁷R⁸R⁹) or —SO₂—R¹⁰—Si(R⁷R⁸R⁹),
R³ is
   (i) hydrogen, hydroxy, halogen, cyano, —CF₃, C₁-C₆-alkoxy —OCF₃ or —NR¹¹R¹², or
   (ii) a C₁-C₆-alkyl radical which is optionally substituted one or more times, identically or differently, by halogen, hydroxy, C₁-C₆-alkoxy or the group —NR¹¹R¹², or
   (iii) a C₁-C₆-alkoxy group which is optionally substituted one or more times, identically and/or differently, by halogen, hydroxy, C₁-C₆-alkoxy or the group —NR¹¹R¹², or
   (iv) a C₃-C₇-cycloalkyl ring which is optionally substituted one or more times, identically or differently, by halogen, hydroxy, C₁-C₆-alkoxy, the group —NR¹¹R¹² and/or C₁-C₆-alkyl,
R⁴ is
   (i) halogen, cyano, nitro, —NR¹¹R¹², —CF₃, C₁-C₆-alkoxy or —OCF₃ or
   (ii) a C₁-C₆-alkyl, C₂-C₆-alkenyl or C₂-C₆-alkynyl radical which is optionally substituted one or more times, identically or differently, by hydroxy, —NR¹¹R¹², cyano, halogen, C₁-C₆-alkoxy, —CF₃ and/or —OCF₃, or
   (iii) a C₆-aryl ring which is optionally substituted one or more times, identically or differently, by hydroxy, —NR¹¹R¹², cyano, halogen, —CF₃, C₁-C₆-alkoxy, —OCF₃ and/or C₁-C₆-alkyl, or
   (iv) a heteroaryl ring which is optionally substituted one or more times, identically or differently, by hydroxy, —NR¹¹R¹², cyano, halogen, —CF₃, C₁-C₆-alkoxy, —OCF₃ and/or C₁-C₆-alkyl and has 5 or 6 ring atoms,
X is —S—, —S(O)—, —NH— or —O—,
Y is —NR¹³—, —S—, —S(O)—, or —O—,
   where
   R¹⁵ may be a
      (i) C₁-C₆-alkyl radical or
      (ii) C₃-C₇-cycloalkyl radical or
      (iii) C₃-C₆-alkenyl radical or
      (iv) C₃-C₆-alkynyl radical or
      (v) C₆-aryl ring or
      (vi) heteroaryl ring having 5 or 6 ring atoms, each of which may optionally be substituted one or more times, identically or differently, by hydroxy, —NR¹¹R¹², cyano, halogen, —CF₃, C₁-C₆-alkoxy and/or —OCF₃,
   R⁶ may be a
      (i) C₁-C₆-alkyl radical or
      (ii) C₃-C₇-cycloalkyl radical or
      (iii) C₂-C₆-alkenyl radical or
      (iv) C₂-C₆-alkynyl radical or
      (v) C₆-aryl ring or
      (vi) heteroaryl ring having 5 or 6 ring atoms, each of which may optionally be substituted one or more times, identically or differently, by hydroxy, —NR¹¹R¹², cyano, halogen, —CF₃, C₁-C₆-alkoxy and/or —OCF₃,
   R⁷, R⁸
   and R⁹ may be independently of one another
      (i) a C₁-C₆-alkyl radical, and/or
      (ii) a C₆-aryl ring,
   R¹⁰ is a C₁-C₃-alkylene group, and
   R¹¹ and R¹² may be independently of one another
      (i) hydrogen and/or
      (ii) a C₁-C₆-alkyl radical, a C₃-C₇-cycloalkyl radical, a C₂-C₆-alkenyl radical, and/or
      (iii) a C₆-aryl ring and/or
      (iv) a heteroaryl ring having 5 or 6 ring atoms, where (ii), (iii) and (iv) may optionally be substituted one or more times, identically or differently, by hydroxy, —NR¹³R¹⁴, cyano, halogen, —CF₃, C₁-C₆-alkoxy and/or —OCF₃, or
   R¹¹ and R¹² together with the nitrogen atom form a heterocyclyl ring which has 3 to 7 ring atoms and may optionally be substituted one or more times, identically or differently, by hydroxy, —NR¹³R¹⁴, cyano, halogen, —CF₃, C₁-C₆-alkoxy and/or —OCF₃ and may comprise a further heteroatom, and
   R¹³ and R¹⁴ are independently of one another hydrogen or a C₁-C₆-alkyl radical which is optionally substituted one or more times, identically or differently, by hydroxy, —NH₂, cyano, halogen, —CF₃, C₁-C₆-alkoxy and/or —OCF₃,
      and the salts, diastereomers and enantiomers thereof.

The following definitions underlie the present application:

Cₙ-Alkyl Radical:

Monovalent, straight-chain or branched, saturated hydrocarbon radical having n carbon atoms.

A C₁-C₆ alkyl radical includes inter alia for example:
methyl-, ethyl-, propyl-, butyl-, pentyl-, hexyl-, iso-propyl-, iso-butyl-, sec-butyl-, tert-butyl-, iso-pentyl-, 2-methylbutyl-, 1-methylbutyl-, 1-ethylpropyl-, 1,2-dimethylpropyl-, neo-pentyl-, 1,1-dimethylpropyl-, 4-methylpentyl-, 3-methylpentyl-, 2-methylpentyl-, 1-methylpentyl-, 2-ethylbutyl-, 1-ethylbutyl-, 3,3-dimethylbutyl-, 2,2-dimethylbutyl-, 1,1-dimethylbutyl-, 2,3-dimethylbutyl-, 1,3-dimethylbutyl-1,2-dimethylbutyl-.

A methyl, ethyl or propyl radical is preferred.

The alkyl radical may optionally be substituted one or more times, identically or differently, by hydroxy, —NR¹¹R¹², cyano, halogen, —CF₃, C₁-C₆-alkoxy and/or —OCF₃.

hydroxy is preferred.

Cₙ-Alkenyl Radical:

monovalent, straight-chain or branched hydrocarbon radical having n carbon atoms and at least one double bond.

A C₂-C₆ alkenyl radical includes inter alia for example:
vinyl-, allyl-, (E)-2-methylvinyl-, (Z)-2-methylvinyl-, homoallyl-, (E)-but-2-enyl-, (Z)-but-2-enyl-, (E)-but-1-enyl-, (Z)-but-1-enyl-, pent-4-enyl-, (E)-pent-3-enyl-, (Z)-pent-3-enyl-, (E)-pent-2-enyl-, (Z)-pent-2-enyl-, (E)-pent-1-enyl-, (Z)-pent-1-enyl-, hex-5-enyl-, (E)-hex-4-enyl-, (Z)-hex-4-enyl-, (E)-hex-3-enyl-, (Z)-hex-3-enyl-, (E)-hex-2-enyl-, (Z)-hex-2-enyl-, (E)-hex-1-enyl-, (Z)-hex-1-enyl-, isopropenyl-, 2-methylprop-2-enyl-, 1-methylprop-2-enyl-, 2-methylprop-1-enyl-, (E)-1-methylprop-1-enyl-, (Z)-1-methylprop-1-enyl-, 3-methyl but-3-enyl-, 2-methylbut-3-enyl-, 1-methylbut-3-enyl-, 3-methylbut-2-enyl-, (E)-2-methylbut-2-enyl-, (Z)-2-methylbut-2-enyl-, (E)-1-methylbut-2-enyl-, (Z)-1-methylbut-2-enyl-, (E)-3-methylbut-1-enyl-, (Z)-3-methylbut-1-enyl-, (E)-2-methylbut-1-enyl-, (Z)-2-methylbut-1-enyl-, (E)-1-methylbut-1-enyl-, (Z)-1-methylbut-1-enyl-, 1,1-dimethylprop-2-enyl-, 1-ethylprop-1-enyl-, 1-propylvinyl-, 1-isopropylvinyl-, 4-methylpent-4-enyl-, 3-methylpent-4-enyl-, 2-methylpent-4-enyl-, 1-methylpent-4-enyl-, 4-methylpent-3-enyl-, (E)-3-methylpent-3-enyl-, (Z)-3-methylpent-3-enyl-, (E)-2-methylpent-3-enyl-, (Z)-2-methylpent-3-enyl-, (E)-1-methylpent-3-enyl-, (Z)-1-methylpent-3-enyl-, (E)-4-methylpent-2-enyl-, (Z)-4-methylpent-2-enyl-, (E)-3-methylpent-2-enyl-, (Z)-3-methylpent-2-enyl-, (E)-2-methylpent-2-enyl-, (Z)-2-methylpent-2-enyl-, (E)-1-methylpent-2-enyl-, (Z)-1-methylpent-2-enyl-, (E)-4-methylpent-1-enyl-, (Z)-4-methylpent-1-enyl-, (E)-3-methylpent-1-enyl-, (Z)-3-methylpent-1-enyl-, (E)-2-methylpent-1-enyl-, (Z)-2-methylpent-1-enyl-, (E)-1-methylpent-1-enyl-, (Z)-1-methylpent-1-enyl-, 3-ethylbut-3-enyl-, 2-ethylbut-3-enyl-, 1-ethylbut-3-enyl-, (E)-3-ethylbut-2-enyl-, (Z)-3-ethylbut-2-enyl-, (E)-2-ethylbut-2-enyl-, (Z)-2-ethylbut-2-enyl-, (E)-1-ethylbut-2-enyl-, (Z)-1-ethylbut-2-enyl-, (E)-3-ethylbut-1-enyl-, (Z)-3-ethylbut-1-enyl-, 2-ethylbut-1-enyl-, (E)-1-ethylbut-1-enyl-, (Z)-1-ethylbut-1-enyl, 2-propylprop-2-enyl-, 1-propylprop-2-enyl-, 2-isopropylprop-2-enyl-, 1-isopropylprop-2-enyl-, (E)-2-propylprop-1-enyl-, (Z)-2-propylprop-1-enyl-, (E)-1-propylprop-1-enyl-, (Z)-1-propylprop-1-enyl-, (E)-2-isopropylprop-1-enyl-, (Z)-2-isopropylprop-1-enyl-, (E)-1-isopropylprop-1-enyl-, (Z)-1-isopropylprop-1-enyl-, (E)-3,3-dimethylprop-1-enyl-, (Z)-3,3-dimethylprop-1-enyl-, 1-(1,1-dimethylethyl)ethenyl.

A vinyl or allyl radical is preferred.

The alkenyl radical may optionally be substituted one or more times, identically or differently, by hydroxy, —NR¹¹R¹², cyano, halogen, —CF₃, C₁-C₆-alkoxy and/or —OCF₃.

Hydroxy is preferred.

Cₙ-Alkynyl Radical:

Monovalent, straight-chain or branched hydrocarbon radical having n carbon atoms and at least one triple bond.

A C₂-C₆ alkynyl radical includes inter alia for example:
ethynyl-, prop-1-ynyl-, prop-2-ynyl-, but-1-ynyl-, but-2-ynyl-, but-3-ynyl-, pent-1-ynyl-, pent-2-ynyl-, pent-3-ynyl-, pent-4-ynyl-, hex-1-ynyl-, hex-2-ynyl-, hex-3-ynyl-, hex-4-ynyl-, hex-5-ynyl-, 1-methylprop-2-ynyl-, 2-methylbut-3-ynyl-, 1-methylbut-3-ynyl-, 1-methylbut-2-ynyl-, 3-methylbut-1-ynyl-, 1-ethylprop-2-ynyl-, 3-methylpent-4-ynyl-, 2-methylpent-4-ynyl-, 1-methylpent-4-ynyl-, 2-methylpent-3-ynyl-, 1-methylpent-3-ynyl-, 4-methylpent-2-ynyl-, 1-methylpent-2-ynyl-, 4-methylpent-1-ynyl-, 3-methylpent-1-ynyl-, 2-ethylbut-3-ynyl-, 1-ethylbut-3-ynyl-, 1-ethylbut-2-ynyl-, 1-propylprop-2-ynyl-, 1-isopropylprop-2-ynyl-, 2,2-dimethyl-but-3-ynyl-, 1,1-dimethylbut-3-ynyl-, 1,1-dimethylbut-2-ynyl- or a 3,3-dimethylbut-1-ynyl-.

An ethynyl, prop-1-ynyl or prop-2-ynyl radical is preferred.

The alkynyl radical may optionally be substituted one or more times, identically or differently, by hydroxy, —NR¹¹R¹², cyano, halogen, —CF₃, C₁-C₆-alkoxy and/or —OCF₃.

Hydroxy is preferred.

Cₙ-Alkylene:

Divalent, straight-chain or branched hydrocarbon group having n carbon atoms.

A C₁-C₆-alkylene group includes inter alia for example:
methylene-(—CH₂—), ethylidene-(—CH(CH₃)—), ethylene-(—CH₂CH₂—), prop-1,3-ylene-(—CH₂CH₂CH₂—), prop-1,2-ylene-(—CH₂CH(CH₃)—), but-1,4-ylene-(—CH₂CH₂CH₂CH₂—), pent-1,5-ylene-(—CH₂CH₂CH₂CH₂CH₂—) or hex-1,6-ylene-(—CH₂CH₂CH₂CH₂CH₂CH₂—).

The alkylene group may optionally be substituted one or more times, identically or differently, by hydroxy, —NR¹¹R¹², cyano, halogen, —CF₃, C₁-C₆-alkoxy and/or —OCF₃.

Hydroxy is preferred.

The following unbranched alkylene groups are provided for B: prop-1,3-ylene-(—CH₂CH₂CH₂—), but-1,4-ylene-(—CH₂CH₂CH₂CH₂—), pent-1,5-ylene-(—CH₂CH₂CH₂CH₂CH₂—) or hex-1,6-ylene-(—CH₂CH₂CH₂CH₂CH₂CH₂—).

A prop-1,3-ylene, but-1,4-ylene or a pent-1,5-ylene group is preferred for B. A but-1,4-ylene group is particularly preferred for B.

The alkylene group B may optionally be substituted one or more times, identically or differently, by hydroxy, —NR¹¹R¹², cyano, halogen, —CF₃, —C₁-C₆-alkoxy, —NR¹³—C(O)—C₁-C₃-alkyl, —NR¹³—SO₂—C₁-C₃-alkyl, —OCF₃ and/or one or more C₁-C₆-alkyl radicals which are optionally substituted one or more times, identically or differently, by hydroxy, —NR¹¹R¹², cyano, halogen, —CF₃, —C₁-C₆-alkoxy, —NR¹³—C(O)—C₁-C₃-alkyl, —NR¹³—SO₂—C₁-C₃-alkyl or —OCF₃.

Hydroxy and C₁-C₆-alkyl radicals which are optionally substituted one or more times, identically or differently, by hydroxy, —NR¹¹R¹², C₁-C₆-alkoxy, —NR¹³—C(O)—C₁-C₃-alkyl or —NR¹³—SO₂—C₁-C₃-alkyl are preferred as substitutents for B.

Cₙ-Cycloalkyl:

Monovalent, cyclic hydrocarbon radical having n carbon atoms.

C₃-C₇-Cycloalkyl ring includes:

cyclopropyl, cyclobutyl, cyclopentyl, cyclohexyl and cycloheptyl.

A cyclopropyl, cyclopentyl or a cyclohexyl ring is preferred.

The cycloalkyl ring may be optionally substituted one or more times, identically or differently, by hydroxy, —NR¹¹R¹², cyano, halogen, —CF₃, C₁-C₆-alkoxy, —OCF₃ and/or C₁-C₆-alkyl.

Cₙ-Alkoxy:

Straight-chain or branched Cₙ-alkyl ether of the formula —OR with R=alkyl.

A Cₙ-alkoxy radical may be substituted one or more times, identically or differently, by halogen, hydroxy, C₁-C₆-alkoxy or the group —NR¹¹R¹².

Cₙ-Aryl

Cₙ-Aryl is a monovalent, aromatic ring system without heteroatom having n carbon atoms.

C₆-Aryl is identical to phenyl.

Phenyl is preferred.

A Cₙ-aryl ring may be substituted one or more times, identically or differently, by hydroxy, —NR¹¹R¹², cyano, halogen, —CF₃, C₁-C₆-alkoxy, —OCF₃ and/or C₁-C₆-alkyl.

Heteroatoms

Heteroatoms are to be understood to include oxygen, nitrogen or sulphur atoms.

Heteroaryl

Heteroaryl is a monovalent, aromatic ring system having at least one heteroatom different from a carbon. Heteroatoms which may occur are nitrogen atoms, oxygen atoms and/or sulphur atoms. The valence bond may be on any aromatic carbon atom or on a nitrogen atom.

Heteroaryl rings having 5 ring atoms include for example the rings:
thienyl, thiazolyl, furanyl, pyrrolyl, oxazolyl, imidazolyl, pyrazolyl, isoxazolyl, isothiazolyl, oxadiazolyl, triazolyl, tetrazolyl and thiadiazolyl.

Heteroaryl rings having 6 ring atoms include for example the rings:
pyridyl, pyridazinyl, pyrimidinyl, pyrazinyl and triazinyl.

A heteroaryl ring having 5 or 6 ring atoms may be substituted one or more times, identically or differently, by hydroxy, —NR¹¹R¹², cyano, halogen, —CF₃, C₁-C₆-alkoxy, —OCF₃ and/or C₁-C₆-alkyl.

Heterocyclyl

Heterocyclyl in the context of the invention is a completely hydrogenated heteroaryl (completely hydrogenated heteroaryl-saturated heterocyclyl), i.e. a non-aromatic ring system having at least one heteroatom different from a carbon. Heteroatoms which may occur are nitrogen atoms, oxygen atoms and/or sulphur atoms. The valence bond may be on any carbon atom or on a nitrogen atom.

Heterocyclyl ring having 3 ring atoms includes for example:

aziridinyl.

Heterocyclyl ring having 4 ring atoms includes for example:

azetidinyl.

Heterocyclyl rings having 5 ring atoms include for example the rings:

pyrrolidinyl, imidazolidinyl and pyrazolidinyl.

Heterocyclyl rings having 6 ring atoms include for example the rings:

piperidinyl, piperazinyl, morpholinyl and thiomorpholinyl.

Heterocyclyl ring having 7 ring atoms includes for example:

azepanyl, [1,3]-diazepanyl, [1,4]-diazepanyl.

A heterocyclyl ring having 3 to 7 ring atoms may be substituted one or more times, identically or differently, by hydroxy, —NR¹¹R¹², cyano, halogen, —CF₃, C₁-C₆-alkoxy, —OCF₃ and/or C₁-C₆-alkyl.

Halogen

The term halogen includes fluorine, chlorine, bromine and iodine.

Bromine is preferred.

The substitutents —NR¹¹R¹² and —NR¹³R¹⁴ are optionally substituted amino groups,
where
R¹¹ and R¹² may be independently of one another
   (i) hydrogen and/or
   (ii) a C₁-C₆-alkyl radical, a C₃-C₇-cycloalkyl radical, a C₂-C₆-alkenyl radical, and/or
   (iii) a C₆-aryl ring and/or
   (iv) a heteroaryl ring having 5 or 6 ring atoms, where (ii), (iii) and (iv) may optionally be substituted one or more times, identically or differently, by hydroxy, —NR¹³R¹⁴, cyano, halogen, —CF₃, C₁-C₆-alkoxy and/or —OCF₃, or
R¹¹ and R¹² together with the nitrogen atom form a heterocyclyl ring which has 3 to 7 ring atoms and may optionally be substituted one or more times, identically or differently, by hydroxy, —NR¹³R¹⁴, cyano, halogen, —CF₃, C₁-C₆-alkoxy and/or —OCF₃ and may comprise a further heteroatom, and
R¹³ and R¹⁴ are independently of one another hydrogen or a C₁-C₆-alkyl radical which is optionally substituted one or more times, identically or differently, by hydroxy, —NH₂, cyano, halogen, —CF₃, C₁-C₆-alkoxy and/or —OCF₃.

R¹¹ and R¹² are preferably independently of one another hydrogen and/or C₁-C₆-alkyl radicals.

B in the general formula I may be:

a prop-1,3-ylene, but-1,4-ylene, pent-1,5-ylene or hex-1,6-ylene group, which may be substituted one or more times, identically or differently, by
(i) hydroxy, —NR¹¹R¹², cyano, halogen, —CF₃, C₁-C₆-alkoxy, —NR¹³—C(O)—C₁-C₃-alkyl, —NR¹³—SO₂—C₁-C₃-alkyl, —OCF₃ and/or
(ii) one or more C₁-C₆-alkyl radicals which are optionally substituted one or more times, identically or differently, by hydroxy, —NR¹¹R¹², cyano, halogen, —CF₃, C₁-C₆-alkoxy, —NR¹³—C(O)—C₁-C₃-alkyl, —NR¹³—SO₂—C₁-C₃-alkyl or —OCF₃.

Substituents preferred for B are hydroxy and/or one or more C₁-C₆-alkyl radicals which are optionally substituted one or more times, identically or differently, by hydroxy, —NR¹¹R¹², cyano, halogen, —CF₃, C₁-C₆-alkoxy, —NR¹³—C(O)—C₁-C₃-alkyl, —NR¹³—SO₂—C₁-C₃-alkyl or —OCF₃.

Substituents particularly preferred for B are hydroxy and/or one or more C₁-C₆-alkyl radicals which are optionally substituted one or more times, identically or differently, by hydroxy, —NR¹¹R¹², C₁-C₆-alkoxy, —NR¹³—C(O)—C₁-C₃-alkyl or —NR¹³—SO₂—C₁-C₃-alkyl.

B is preferably a prop-1,3-ylene, but-1,4-ylene or pent-1,5-ylene group which may be substituted one or more times, identically or differently, by hydroxy and/or one or more C₁-C₆-alkyl radicals which are optionally substituted one or more times, identically or differently, by hydroxy, —NR¹¹R¹², cyano, halogen, —CF₃, C₁-C₆-alkoxy, —NR¹³—C(O)—C₁-C₃-alkyl, —NR¹³—SO₂—C₁-C₃-alkyl or —OCF₃.

B is particularly preferably a but-1,4-ylene group which may be substituted one or more times, identically or differently, by hydroxy and/or one or more C₁-C₆-alkyl radicals which are optionally substituted one or more times, identically or differently, by hydroxy, —NR¹¹R¹², C₁-C₆-alkoxy, —NR¹³—C(O)—C₁-C₃-alkyl or —NR¹³—SO₂—C₁-C₃-alkyl.

R¹ in the general formula I may be:
(i) a C₁-C₆-alkyl radical which is optionally substituted one or more times, identically or differently, by hydroxy, —NR¹¹R¹², cyano, halogen, C₁-C₆-alkoxy, —CF₃ and/or —OCF₃, or
(ii) a C₃-C₇-cycloalkyl ring which is optionally substituted one or more times, identically or differently, by hydroxy, —NR¹¹R¹², cyano, halogen, —CF₃, C₁-C₆-alkoxy, —OCF₃ and/or C₁-C₆-alkyl, or
(iii) a C₆-aryl ring which is optionally substituted one or more times, identically or differently, by hydroxy, —NR¹¹R¹², cyano, halogen, —CF₃, C₁-C₆-alkoxy, —OCF₃ and/or C₁-C₆-alkyl, or
(iv) a heteroaryl ring which is optionally substituted one or more times, identically or differently, by hydroxy, —NR¹¹R¹², cyano, halogen, —CF₃, C₁-C₆-alkoxy, —OCF₃ and/or C₁-C₆-alkyl and has 5 or 6 ring atoms.

R¹ is preferably a C₁-C₆-alkyl radical which is optionally substituted one or more times, identically or differently, by hydroxy, —NR¹¹R¹², cyano, halogen, —CF₃, C₁-C₆-alkoxy and/or —OCF₃.

R² in the general formula I may be:

R⁵, —SO₂—R⁶, —C(O)O—R⁶, —C(O)—R⁶, —C(O)—NR¹¹R¹², —C(S)—NR¹¹R¹², —Si(R⁷R⁸R⁹), —R¹⁰—Si(R⁷R⁸R⁹) or —SO₂—R¹⁰—Si(R⁷R⁸R⁹),
where
R⁵ may be a
   (i) C₁-C₆-alkyl radical or
   (ii) C₃-C₆-alkenyl radical or
   (iii) C₃-C₆-alkynyl radical or
   (iv) C₆-aryl ring or
   (v) heteroaryl ring having 5 or 6 ring atoms, each of which may optionally be substituted one or more times, identically or differently, by hydroxy, —NR¹¹R¹², cyano, halogen, —CF₃, C₁-C₆-alkoxy and/or —OCF₃,
R⁶ may be a
   (i) C₁-C₆-alkyl radical or
   (ii) C₂-C₆-alkenyl radical or
   (iii) C₂-C₆-alkynyl radical or
   (iv) C₆-aryl ring or
   (v) heteroaryl ring having 5 or 6 ring atoms, each of which may optionally be substituted one or more times, identically or differently, by hydroxy, —NR¹¹R¹², cyano, halogen, —CF₃, C₁-C₆-alkoxy and/or —OCF₃,
R⁷, R⁸
and R⁹ may be independently of one another
   (i) a C₁-C₆-alkyl radical, and/or
   (ii) a C₆-aryl ring,
R¹⁰ is a C₁-C₃-alkylene group,
R¹¹ and R¹² may be independently of one another
   (i) hydrogen and/or
   (ii) a C₁-C₆-alkyl radical, a C₃-C₇-cycloalkyl radical, a C₂-C₆-alkenyl radical, and/or
   (iii) a C₆-aryl ring and/or
   (iv) a heteroaryl ring having 5 or 6 ring atoms, where (ii), (iii) and (iv) may optionally be substituted one or more times, identically or differently, by hydroxy, —NR¹³R¹⁴, cyano, halogen, —CF₃, C₁-C₆-alkoxy and/or —OCF₃, or
R¹¹ and R¹² together with the nitrogen atom form a heterocyclyl ring which has 3 to 7 ring atoms and may optionally be substituted one or more times, identically or differently, by hydroxy, —NR¹³R¹⁴, cyano, halogen, —CF₃, C₁-C₆-alkoxy and/or —OCF₃ and may comprise a further heteroatom, and
R¹³ and R¹⁴ are independently of one another hydrogen or a C₁-C₆-alkyl radical which is optionally substituted one or more times, identically or differently, by hydroxy, —NH₂, cyano, halogen, —CF₃, C₁-C₆-alkoxy and/or —OCF₃.

R² is preferably R⁵, —SO₂—R⁶, —C(O)O—R⁶, —C(O)—R⁶, —C(O)—NR¹¹R¹² or —SO₂—R¹⁰—Si(R⁷R⁸R⁹), where
R⁵, R⁶, R⁷,
R⁸ and R⁹ is independently of one another a C₁-C₆-alkyl radical which is optionally substituted one or more times, identically or differently, by hydroxy, —NR¹¹R¹², cyano, halogen, —CF₃, C₁-C₆-alkoxy and/or —OCF₃,
R¹⁰ is a C₁-C₃-alkylene group, and
R¹¹ and R¹² may be independently of one another
   (i) hydrogen and/or
   (ii) a C₁-C₆-alkyl radical,
   where (ii) is optionally substituted one or more times, identically or differently, by hydroxy,
   —NR¹³R¹⁴, cyano, halogen, —CF₃, C₁-C₆-alkoxy and/or —OCF₃, where
R¹³ and R¹⁴ may be independently of one another hydrogen or a C₁-C₆-alkyl radical which is optionally substituted one or more times, identically or differently, by hydroxy, —NH₂, cyano, halogen, —CF₃ and/or —OCF₃.

Likewise preferred for R² is:

—SO₂—R⁶, —C(O)O—R⁶, —C(O)—R⁶, —C(O)—NR¹¹R¹² or —SO₂—R¹⁰—Si(R⁷R⁸R⁹),

where
R⁶ is a C₁-C₆-alkyl radical,
R⁷, R⁸
and R⁹ may be independently of one another a C₁-C₆-alkyl radical,
R¹⁰ is a C₁-C₃-alkylene group,
R¹¹ and R¹² may be independently of one another
   (i) hydrogen and/or
   (ii) a C₁-C₆-alkyl radical, a C₃-C₇-cycloalkyl radical, a C₂-C₆-alkenyl radical, and/or
   (iii) a C₆-aryl ring and/or
   (iv) a heteroaryl ring having 5 or 6 ring atoms, where (ii), (iii) and (iv) may optionally be substituted one or more times, identically or differently, by hydroxy, —NR¹³R¹⁴, cyano, halogen, —CF₃, C₁-C₆-alkoxy and/or —OCF₃,
R¹³ and R¹⁴ are independently of one another a C₁-C₆-alkyl radical.

R² is particularly preferably:

—SO₂—R⁶, —C(O)O—R⁶, —C(O)—NR¹¹R¹² or —SO₂—R¹⁰—Si(R⁷R⁸R⁹),

where

R⁶, R⁷, R⁸ and R⁹ are independently of one another C₁-C₅-alkyl radicals,

R¹⁰ is a C₁-C₅-alkylene group, and

R¹¹ and R¹² may be independently of one another hydrogen and/or C₁-C₆-alkyl radicals.

R³ in the general formula I may be:
(i) hydrogen, hydroxy, halogen, cyano, —CF₃, C₁-C₆-alkoxy —OCF₃ or —NR¹¹R¹² or
(ii) a C₁-C₆-alkyl radical which is optionally substituted one or more times, identically or differently, by halogen, hydroxy, C₁-C₆-alkoxy or the group —NR¹¹R¹², or
(iii) a C₁-C₆-alkoxy group which is optionally substituted one or more times, identically and/or differently, by halogen, hydroxy, C₁-C₆-alkoxy or the group —NR¹¹R¹², or
(iv) a C₃-C₇-cycloalkyl ring which is optionally substituted one or more times, identically or differently, by halogen, hydroxy, C₁-C₆-alkoxy, the group —NR¹¹R¹² and/or C₁-C₆-alkyl,
where
R¹¹ and R¹² may be independently of one another
   (i) hydrogen and/or
   (ii) a C₁-C₆-alkyl radical, and/or
   (iii) a C₆-aryl ring and/or
   (iv) a heteroaryl ring having 5 or 6 ring atoms, where (ii), (iii) and (iv) may optionally be substituted one or more times, identically or differently, by hydroxy,
      —NR¹³R¹⁴, cyano, halogen, —CF₃, C₁-C₆-alkoxy and/or —OCF₃, or
R¹¹ and R¹² together with the nitrogen atom form a heterocyclyl ring which has 3 to 7 ring atoms and may optionally be substituted one or more times, identically or differently, by hydroxy, —NR¹²R¹⁴, cyano, halogen, —CF₃, C₁-C₆-alkoxy and/or —OCF₃, and may comprise a further heteroatom, and
R¹³ and R¹⁴ are independently of one another hydrogen or a C₁-C₆-alkyl radical which is optionally substituted one or more times, identically or differently, by hydroxy, —NH₂, cyano, halogen, —CF₃, C₁-C₆-alkoxy and/or —OCF₃.

R³ is preferably:
(i) hydrogen, hydroxy, halogen, C₁-C₆alkoxy, —NR¹¹R¹²
(ii) a —C₁-C₆-alkyl radical which is optionally substituted one or more times, identically or differently, by halogen, hydroxy, C₁-C₆-alkoxy or the group —NR¹¹R¹² or or
(iii) a C₁-C₆-alkoxy group which is optionally substituted one or more times, identically and/or differently, by halogen, hydroxy, C₁-C₆-alkoxy or the group —NR¹¹R¹².

R³ is particularly preferably hydrogen.

R⁴ in the general formula I may be:
(i) halogen, cyano, nitro, —NR¹¹R¹², —CF₃, C₁-C₆-alkoxy or —OCF₃ or
(ii) a C₁-C₆-alkyl, C₂-C₆-alkenyl or C₂-C₆-alkynyl radical which is optionally substituted one or more times, identically or differently, by hydroxy, —NR¹¹R¹², cyano, halogen, C₁-C₆-alkoxy, —CF₃ and/or —OCF₃ or
(iii) a C₆-aryl ring which is optionally substituted one or more times, identically or differently, by hydroxy, —NR¹¹R¹², cyano, halogen, —CF₃, C₁-C₆-alkoxy, —OCF₃ and/or C₁-C₆-alkyl or
(iv) a heteroaryl ring which is optionally substituted one or more times, identically or differently, by hydroxy, —NR¹¹R¹², cyano, halogen, —CF₃, C₁-C₆-alkoxy, —OCF₃ and/or C₁-C₆-alkyl and has 5 or 6 ring atoms,
   where
   R¹¹ and R¹² may be independently of one another
      (i) hydrogen and/or
      (ii) a C₁-C₆-alkyl radical, and/or
      (iii) a C₆-aryl ring and/or
      (iv) a heteroaryl ring having 5 or 6 ring atoms, where (ii), (iii) and (iv) may optionally be substituted one or more times, identically or differently, by hydroxy, —NR¹³R¹⁴, cyano, halogen, —CF₃, C₁-C₆-alkoxy and/or —OCF₃, or
   R¹¹ and R¹² together with the nitrogen atom form a heterocyclyl ring which has 3 to 7 ring atoms and may optionally be substituted one or more times, identically or differently, by hydroxy, —NR¹³R¹⁴, cyano, halogen, —CF₃, C₁-C₆-alkoxy and/or —OCF₃ and may comprise a further heteroatom, and
   R¹³ and R¹⁴ are independently of one another hydrogen or a C₁-C₆-alkyl radical which is optionally substituted one or more times, identically or differently, by hydroxy, —NH₂, cyano, halogen, —CF₃, C₁-C₆-alkoxy and/or —OCF₃.

R⁴ is preferably:
(i) halogen or —CF₃
(ii) a C₆-aryl ring which is optionally substituted one or more times, identically or differently, by hydroxy, —NR¹¹R¹², cyano, halogen, —CF₃, C₁-C₆-alkoxy, —OCF₃ and/or C₁-C₆-alkyl, or
(iii) a heteroaryl ring which is optionally substituted one or more times, identically or differently, by hydroxy, —NR¹¹R¹², cyano, halogen, —CF₃, C₁-C₆-alkoxy, —OCF₃ and/or C₁-C₆-alkyl and has 5 or 6 ring atoms, where
   R¹¹ and R¹² may be independently of one another
      (i) hydrogen and/or
      (ii) a C₁-C₆-alkyl radical,
      where (ii) is optionally substituted one or more times, identically or differently, by hydroxy, —NR¹³R¹⁴, cyano, halogen, —CF₃, C₁-C₆-alkoxy and/or —OCF₃, and
   R¹³ and R¹⁴ are independently of one another hydrogen or a C₁-C₆-alkyl radical which is optionally substituted one or more times, identically or differently, by hydroxy, —NH₂, cyano, halogen, —CF₃, C₁-C₆-alkoxy and/or —OCF₃.

R⁴ is particularly preferably halogen, in particular bromine or iodine.

X in the general formula I may be:

—S—, —S(O)—, —NH— or —O—.

X is preferably —NH— or —O—.

Y in the general formula I may be:—

—NR¹³—, —S—, —S(O)—, or —O—,

where R¹³ may be hydrogen or a C₁-C₆-alkyl radical which is optionally substituted one or more times, identically or differently, by hydroxy, —NH₂, cyano, halogen, —CF₃, C₁-C₆-alkoxy and/or —OCF₃.

Y is preferably —NH— or —S—, particularly preferably —NH—.

R⁵ in the general formula I may be:
(i) a C₁-C₆-alkyl radical or
(ii) a C₃-C₆-alkenyl radical or
(iii) a C₃-C₆-alkynyl radical or
(iv) a C₆-aryl ring or
(v) a heteroaryl ring having 5 or 6 ring atoms,
   each of which may optionally be substituted one or more times, identically or differently, by hydroxy, —NR¹¹R¹², cyano, halogen, —CF₃, C₁-C₆-alkoxy and/or —OCF₃.

R⁵ is preferably a C₁-C₆-alkyl or a C₃-C₆-alkenyl radical, each of which may optionally be substituted one or more times, identically or differently, by hydroxy, —NR¹¹R¹², cyano, halogen, —CF₃, C₁-C₆-alkoxy and/or —OCF₃.

R⁵ is particularly preferably a C₂-C₅-alkyl radical which may optionally be substituted one or more times, identically or differently, by hydroxy, —NR¹¹R¹² and/or C₁-C₆-alkoxy.

R⁶ in the general formula I may be:
(i) a C₁-C₆-alkyl radical or
(ii) a C₂-C₆-alkenyl radical or
(iii) a C₂-C₆-alkynyl radical or
(iv) a C₆-aryl ring or
(v) a heteroaryl ring having 5 or 6 ring atoms,
   each of which may optionally be substituted one or more times, identically or differently, by hydroxy, —NR¹¹R¹², cyano, halogen, —CF₃, C₁-C₆-alkoxy and/or —OCF₃.

R⁶ is preferably a C₁-C₆-alkyl radical which may optionally be substituted one or more times, identically or differently, by hydroxy, —NR¹¹R¹², cyano, halogen, —CF₃, C₁-C₆-alkoxy and/or —OCF₃.

R⁶ is particularly preferably a C₂-C₅-alkyl radical which may optionally be substituted one or more times, identically or differently, by hydroxy, —NR¹¹R¹², and/or C₁-C₆-alkoxy.

R⁷, R⁸ and R⁹ in the general formula I may be independently of one another:
(i) a C₁-C₆-alkyl radical, and/or
(ii) a C₆-aryl ring.

C₁-C₆-alkyl radicals are preferred for R⁷, R⁸ and R⁹.

R¹⁰ in the general formula I may be:

a C₁-C₃-alkylene group.

R¹⁰ is preferably ethylene.

R¹¹ and R¹² in the general formula I may be independently of one another:
(i) hydrogen and/or
(ii) a C₁-C₆-alkyl radical, a C₃-C₇-cycloalkyl radical, a C₂-C₆-alkenyl radical, and/or
(iii) a C₆-aryl ring and/or
(iv) a heteroaryl ring having 5 or 6 ring atoms,
   where (ii), (iii) and (iv) may optionally be substituted one or more times, identically or differently, by hydroxy, —NR¹³R¹⁴, cyano, halogen, —CF₃, C₁-C₆-alkoxy and/or —OCF₃, or

R¹¹ and R¹² together with the nitrogen atom form a heterocyclyl ring which has 3 to 7 ring atoms and may optionally be substituted one or more times, identically or differently, by hydroxy, —NR¹³R¹⁴, cyano, halogen, —CF₃, C₁-C₆-alkoxy and/or —OCF₃ and may comprise a further heteroatom, and where

R¹³ and R¹⁴ are independently of one another hydrogen or a C₁-C₆-alkyl radical which is optionally substituted one or more times, identically or differently, by hydroxy, —NH₂, cyano, halogen, —CF₃, C₁-C₆-alkoxy and/or —OCF₃.

R¹¹ and R¹² are preferably independently of one another:
(i) hydrogen and/or
(ii) a C₁-C₆-alkyl radical, and/or
(iii) a C₆-aryl ring and/or
(iv) a heteroaryl ring having 5 or 6 ring atoms,
   where (ii), (iii) and (iv) may optionally be substituted one or more times, identically or differently, by hydroxy, —NR¹³R¹⁴, cyano, halogen, —CF₃, C₁-C₆-alkoxy and/or —OCF₃, or

R¹¹ and R¹² together with the nitrogen atom form a heterocyclyl ring which has 3 to 7 ring atoms and may optionally be substituted one or more times, identically or differently, by hydroxy, —NR¹³R¹⁴, cyano, halogen, —CF₃, C₁-C₆-alkoxy and/or —OCF₃ and may comprise a further heteroatom, and where

R¹³ and R¹⁴ are independently of one another hydrogen or a C₁-C₆-alkyl radical which is optionally substituted one or more times, identically or differently, by hydroxy, —NH₂, cyano, halogen, —CF₃, C₁-C₆-alkoxy and/or —OCF₃.

R¹¹ and R¹² are more preferably independently of one another
(i) hydrogen and/or
(ii) a C₁-C₆-alkyl radical, a C₃-C₇-cycloalkyl radical, a C₂-C₆-alkenyl radical, and/or
(iii) a C₆-aryl ring and/or
(iv) a heteroaryl ring having 5 or 6 ring atoms, where (ii), (iii) and (iv) may optionally be substituted one or more times, identically or differently, by hydroxy, —NR¹³R¹⁴, cyano, halogen, —CF₃, C₁-C₆-alkoxy and/or —OCF₃ and R¹³ and R¹⁴ are independently of one another C₁-C₆-alkyl radicals.

R¹¹ and R¹² are preferably independently of one another hydrogen and/or C₁-C₆-alkyl radicals.

R¹³ and R¹⁴ may be independently of one another:

hydrogen or a C₁-C₆-alkyl radical which is optionally substituted one or more times, identically or differently, by hydroxy, —NH₂, cyano, halogen, —CF₃, C₁-C₆-alkoxy and/or —OCF₃.

R¹³ and R¹⁴ are preferably independently of one another hydrogen and/or a C₁-C₆-alkyl radical.

A preferred subgroup is formed by compounds of the general formula I according to Claim 1

in which
B is a but-1,4-ylene group,
R¹ is a C₁-C₆-alkyl radical,
R² is —SO₂—R⁶, —C(O)O—R⁶, —C(O)—R⁶, —C(O)—NR¹¹R¹² or —SO₂—R¹⁰—Si(R⁷R⁸R⁹),
R³ is hydrogen,
R⁴ is halogen or a heteroaryl ring having 5 or 6 ring atoms,
X is —NH— or —O—,
Y is —NR¹³—,
   where
   R⁶ is a C₁-C₆-alkyl, radical
   R⁷, R⁸
   and R⁹ may independently of one another be a C₁-C₆-alkyl radical,
   R¹⁰ is a C₁-C₃-alkylene group,
   R¹¹ and R¹² may be independently of one another
      (i) hydrogen and/or
      (ii) a C₁-C₆-alkyl radical, a C₃-C₇-cycloalkyl radical, a C₂-C₆-alkenyl radical and/or
      (iii) a C₆-aryl ring and/or
      (iv) a heteroaryl ring having 5 or 6 ring atoms, where (ii), (iii) and (iv) may optionally be substituted one or more times, identically or differently, by hydroxy, —NR¹³R¹⁴, cyano, halogen, —CF₃, C₁-C₆-alkoxy and/or —OCF₃,
R¹³ and R¹⁴ are independently of one another hydrogen and/or a C₁-C₆-alkyl radical,
   and the salts, diastereomers and enantiomers thereof.

A likewise preferred subgroup is formed by compounds according to general formula I

in which

B is a prop-1,3-ylene, but-1,4-ylene, pent-1,5-ylene or hex-1,6-ylene group,
R¹ is a C₁-C₅-alkyl radical,
R² is —SO₂—R⁵, —C(O)O—R⁶, —C(O)—NR¹¹R¹² or —SO₂—R¹⁰—Si(R⁷R⁸R⁹),
   where
   R⁵, R⁶, R⁷,
   R⁸ and R⁹ are independently of one another C₁-C₅-alkyl radicals,
   R¹⁰ is a C₁-C₅-alkylene group,
   R¹¹ and R¹² may be independently of one another hydrogen and/or C₁-C₆-alkyl radicals,
R³ is hydrogen, and
R⁴ is a halogen,
   and the salts, diastereomers and enantiomers thereof.

A particularly preferred subgroup is formed by compounds according to general formula I

in which

B is a but-1,4-ylene group,
R¹ is a methyl group,
R² is an —SO₂—R⁶, —C(O)O—R⁶, —C(O)—NHR⁶ or —SO₂—C₂H₄—Si(CH₃)₃, where R⁶ can be an ethyl or propyl radical,
R³ is hydrogen,
R⁴ is a halogen,
X is —O— or —NH—, and
Y is —NH—,
   and the salts, diastereomers and enantiomers thereof.

Likewise to be regarded as encompassed by the present invention are all compounds which result from every possible combination of the abovementioned possible, preferred and particularly preferred meanings of the substitutents.

Special embodiments of the invention moreover consist of compounds which result from combination of the meanings disclosed directly in the examples for the substitutents.

The following grouping of protein kinases underlies the application:
A. type 1 cell cycle kinases: CDKs, Plk
B. type 2 cell cycle kinases: Aurora
C. angiogenic receptor tyrosine kinases: a) VEGF-R, b) Tie, c) FGF-R, d) EphB4
D. proliferative receptor tyrosine kinases: a) PDGF-R, Flt-3, c-Kit
E. checkpoint kinases: a) ATM/ATR, b) Chk ½, c) TTK/hMps1, BubR1, Bub1
F. anti-apoptotic kinases a) AKT/PKB b) IKK c) PIM1, d) ILK
G. migratory kinases a) FAK, b) ROCK
   A. Type 1 Cell Cycle Kinases: CDK and Plk

The eukaryotic cycle of cell division ensures duplication of the genome and its distribution to the daughter cells by passing through a coordinated and regulated sequence of events. The cell cycle is divided into four consecutive phases: the G1 phase represents the time before DNA replication in which the cell grows and is sensitive to external stimuli. In the S phase, the cell replicates its DNA, and in the G2 phase it prepares itself for entry into mitosis. In mitosis (M phase), the replicated DNA is separated and cell division is completed.

The cyclin-dependent kinases (CDKs), a family of serine/threonine kinases whose members require the binding of a cyclin (Cyc) as regulatory subunit for their activation, drive the cell through the cell cycle. Different CDK/Cyc pairs are active in the different phases of the cell cycle. CDK/Cyc pairs which are important for the basic function of the cell cycle are, for example, CDK4(6)/CycD, CDK2/CycE, CDK2/CycA, CDK1/CycA and CDK1/CycB.

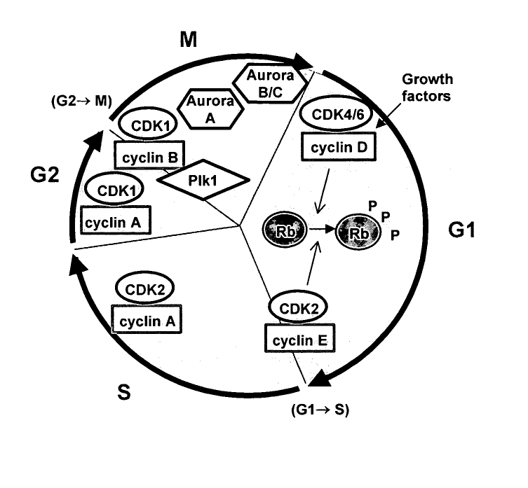

Entry into the cell cycle and passing through the restriction point, which marks the independence of a cell from further growth signals for completion of the initiated cell division, are controlled by the activity of the CDK4(6)/CycD and CDK2/CycE complexes. The essential substrate of these CDK complexes is the retinoblastoma protein (Rb), the product of the retinoblastoma tumour suppressor gene. Rb is a transcriptional corepresssor protein. Besides other mechanisms which are still substantially not understood, Rb binds and inactivates transcription factors of the E2F type, and forms transcriptional repressor complexes with histone deacetylases (HDAC) (Zhang H. S. et al. (2000). Exit from G1 and S phase of the cell cycle is regulated by repressor complexes containing HDAC-Rb-hSWI/SNF and Rb-hSWI/SNF. Cell 101, 79-89). Phosphorylation of Rb by CDKs releases bound E2F transcription factors which lead to transcriptional activation of genes whose products are required for DNA synthesis and progression through the S phase. An additional effect of Rb phosphorylation is to break up Rb-HDAC complexes, thus activating further genes. Phosphorylation of Rb by CDKs is to be equated with going beyond the restriction point. The activity of CDK2/CycE and CDK2/CycA complexes is necessary for progression through the S phase and completion thereof. After replication of the DNA is complete, the CDK1 in the complex with CycA or CycB controls the passing through of the G2 phase and the entry of the cell into mitosis (FIG. 1). In the transition from the G2 phase into mitosis, the polo-like kinase Plk1 contributes to activating CDK1. While mitosis is in progress, Plk1 is further involved in the maturation of the centrosomes, the construction of the spindle apparatus, the segregation of the chromosomes and the separation of the daughter cells.

B. Type 2 Cell Cycle Kinases: Aurora Kinases

The family of Aurora kinases consists in the human body of three members: Aurora-A, Aurora-B and Aurora-C. The Aurora kinases regulate important processes during cell division (mitosis).

Aurora-A is localized on the centrosomes and the spindle microtubules, where it phosphorylates various substrate proteins, inter alia kinesin Eg5, TACC, PP1. The exact mechanisms of the generation of the spindle apparatus and the role of Aurora-A therein are, however, still substantially unclear.

Aurora-B is part of a multiprotein complex which is localized on the centrosome structure of the chromosomes and, besides Aurora-B, comprises, inter alia, INCENP, survivin and borealin/dasra B (summarizing overview in: Vagnarelli & Earnshaw, Chromosomal passengers: the four-dimensional regulation of mitotic events. Chromosoma. 2004 November; 113(5):211-22. Epub 2004 Sep. 4). The kinase activity of Aurora-B ensures that all the connections to the microtubulin spindle apparatus are correct before division of the pairs of chromosomes (so-called spindle checkpoint). Substrates of Aurora-B are in this case, inter alia, histone H3 and MCAK. After separation of the chromosomes, Aurora-B alters its localization and can be found during the last phase of mitosis (cytokinesis) on the still remaining connecting bridge between the two daughter cells. Aurora-B regulates the severance of the daughter cells through phosphorylation of its substrates MgcRacGAP, vimentin, desmin, the light regulatory chain of myosin, and others.

Aurora-C is very similar in its amino acid sequence, localization, substrate specificity and function to Aurora-B (Li X et al. Direct association with inner centromere protein (INCENP) activates the novel chromosomal passenger protein, Aurora-C. J Biol. Chem. 2004 Nov. 5; 279(45):47201-11. Epub 2004 Aug. 16; Chen et al. Overexpression of an Aurora-C kinase-deficient mutant disrupts the Aurora-B/INCENP complex and induces polyploidy. J Biomed Sci. 2005; 12(2):297-310; Yan X et al. Aurora-C is directly associated with Survivin and required for cytokinesis. Genes to ells 2005 10, 617-626). The chief difference between Aurora-B and Aurora-C is the strong overexpression of Aurora-C in the testis (Tseng T C et al. Protein kinase profile of sperm and eggs: cloning and characterization of two novel testis-specific protein kinases (AIE1, AIE2) related to yeast and fly chromosome segregation regulators. DNA Cell Biol. 1998 October; 17(10):823-33.).

The essential function of the Aurora kinases in mitosis makes them target proteins of interest for the development of small inhibitory molecules for the treatment of cancer or other disorders which are caused by disturbances of cell proliferation. Convincing experimental data indicate that inhibition of the Aurora kinases in vitro and in vivo prevents the advance of cellular proliferation and induces programmed cell death (apoptosis). It has been possible to show this by means of (1) siRNA technology (Du & Hannon. Suppression of p160ROCK bypasses cell cycle arrest after Aurora-A/STK15 depletion. Proc Natl Acad Sci U S A. 2004 Jun. 15; 101(24):8975-80. Epub 2004 Jun. 3; Sasai K et al. Aurora-C kinase is a novel chromosomal passenger protein that can complement Aurora-B kinase function in mitotic cells. Cell Motil Cytoskeleton. 2004 December; 59(4):249-63) or (2) overexpression of a dominant-negative Aurora kinase (Honda et al. Exploring the functional interactions between Aurora B, INCENP, and survivin in mitosis. Mol Biol Cell. 2003 August; 14(8):3325-41. Epub 2003 May 29), and (3) with small chemical molecules which specifically inhibit Aurora kinases (Hauf S et al. The small molecule Hesperadin reveals a role for Aurora B in correcting kinetochore-microtubule attachment and in maintaining the spindle assembly checkpoint. J. Cell Biol. 2003 Apr. 28; 161(2):281-94. Epub 2003 Apr. 21.; Ditchfield C et al. Aurora B couples chromosome alignment with anaphase by targeting BubR1, Mad2, and Cenp-E to kinetochores. J. Cell Biol. 2003 Apr. 28; 161(2):267-80.).

Inactivation of Aurora kinases leads to (1) faulty or no development of the mitotic spindle apparatus (predominantly with Aurora-A inhibition) and/or (2) faulty or no separation of the sister chromatids through blocking of the spindle checkpoint (predominantly with Aurora-B/-C inhibition) and/or (3) incomplete separation of daughter cells (predominantly with Aurora-B/-C inhibition). These consequences (1-3) of the inactivation of Aurora kinases singly or as combinations lead eventually to aneuploidy and/or polyploidy and ultimately, immediately or after repeated mitoses, to a non-viable state or to programmed cell death of the proliferating cells (mitotic catastrophe).

Specific kinase inhibitors are able to influence the cell cycle at various stages. Thus, for example, blockade of the cell cycle in the G1 phase or in the transition from the G1 phase to the S phase is to be expected with a CDK4 or a CDK2 inhibitor (type 1 cell cycle kinases). In order now to be able to exploit the advantages of inhibition of Aurora kinases, such as the initiation of aberrant mitoses which lead to cell death, it is necessary for Aurora inhibitors to have a selectivity in relation to type 1 cell cycle kinases.

C. Angiogenic Receptor Tyrosine Kinases

Receptor tyrosine kinases and their ligands are crucial participants in a large number of cellular processes involved in the regulation of the growth and differentiation of cells. Of particular interest here are the vascular endothelial growth factor (VEGF)/VEGF receptor system, the fibroblast growth factor (FGF)/FGF receptor system, the Eph ligand/Eph receptor system, and the Tie ligand/Tie receptor system. In pathological situations associated with an increased formation of new blood vessels (neovascularization) such as, for example, neoplastic diseases, an increased expression of angiogenic growth factors and their receptors has been found. Inhibitors of the VEGF/VEGF receptor system, FGF/FGF receptor system (Rousseau et al., The tyrp1-Tag/tyrp1-FGFR1-DN bigenic mouse: a model for selective inhibition of tumor development, angiogenesis, and invasion into the neural tissue by blockade of fibroblast growth factor receptor activity. Cancer Res. 64,: 2490, 2004), of the EphB4 system (Kertesz et al., The soluble extracellular domain of EphB4 (sEphB4) antagonizes EphB4-EphrinB2 interaction, modulates angiogenesis and inhibits tumor growth. Blood. 2005 Dec. 1; [Epub ahead of print]), and of the Tie ligand/Tie system (Siemeister et al., Two independent mechanisms essential for tumor angiogenesis: inhibition of human melanoma xenograft growth by interfering with either the vascular endothelial growth factor receptor pathway or the Tie-2 pathway. Cancer Res. 59, 3185, 1999) are able to inhibit the development of a vascular system in tumours, thus cut the tumour off from the oxygen and nutrient supply, and therefore inhibit tumour growth.

D. Proliferative Receptor Tyrosine Kinases

Receptor tyrosine kinases and their ligands are crucial participants in the proliferation of cells. Of particular interest here are the platelet-derived growth factor (PDGF) ligand/PDGF receptor system, c-kit ligand/c-kit receptor system and the FMS-like tyrosine kinase 3 (Flt-3) ligand/Flt-3 system. In pathological situations associated with an increased growth of cells such as, for example, neoplastic diseases, an increased expression of proliferative growth factors and their receptors or kinase-activating mutations has been found. Inhibition of the enzymic activity of these receptor tyrosine kinases leads to a reduction of tumour growth. It has been possible to show this for example by studies with the small chemical molecule STI571/Glivec which inhibits inter alia PDGF-R and c-kit (summarizing overviews in: Oestmann A., PDGF receptors—mediators of autocrine tumor growth and regulators of tumor vasculature and stroma, Cytokine Growth Factor Rev. 2004 August; 15(4):275-86; Roskoski R., Signaling by Kit protein-tyrosine kinase—the stem cell factor receptor. Biochem Biophys Res Commun. 2005 Nov. 11; 337(1):1-13.; Markovic A. et al., FLT-3: a new focus in the understanding of acute leukemia. Int J Biochem Cell Biol. 2005 June; 37(6):1168-72. Epub 2005 Jan. 26.).

E. Checkpoint Kinases

Checkpoint kinases mean in the context of the present application cell cycle kinases which monitor the ordered progression of cell division, such as, for example, ATM and ATR, Chk1 and Chk2, Mps1, Bub1 and BubR1. Of particular importance are the DNA damage checkpoint in the G2 phase and the spindle checkpoint during mitosis.

The ATM, ATR, Chk1 and Chk2 kinases are activated by DNA damage to a cell, which activation and leads to arrest of the cell cycle in the G2 phase through inactivation of CDK1. (Chen & Sanchez, Chk1 in the DNA damage response: conserved roles from yeasts to mammals. DNA Repair 3, 1025, 2004). Inactivation of Chk1 causes loss of the G2 arrest induced by DNA damage, thereby leads to progression of the cell cycle in the presence of damaged DNA, and finally leads to cell death (Takai et al. Aberrant cell cycle checkpoint function and early embryonic death in Chk1 (−/−) mice. Genes Dev. 2000 Jun. 15; 14(12):1439-47; Koniaras et al. Inhibition of Chk1-dependent G2 DNA damage checkpoint radiosensitizes p53 mutant human cells. Oncogene. 2001 Nov. 8; 20(51):7453-63.; Liu et al. Chk1 is an essential kinase that is regulated by Atr and required for the G(2)/M DNA damage checkpoint. Genes Dev. 2000 Jun. 15; 14(12):1448-59.). Inactivation of Chk1, Chk2 or Chk1 and Chk2 prevents the G2 arrest caused by DNA damage and makes proliferating cancer cells more sensitive to DNA-damaging therapies such as, for example, chemotherapy or radiotherapy. Chemotherapies leading to DNA damage are, for example, substances inducing DNA strand breaks, DNA-alkylating substances, topoisomerase inhibitors, Aurora kinase inhibitors, substances which influence the construction of the mitotic spindles, hypoxic stress owing to a limited oxygen supply to a tumour (e.g. induced by anti-angiogenic medicaments such as VEGF kinase inhibitors).

A second essential checkpoint within the cell cycle controls the correct construction and attachment of the spindle apparatus to the chromosomes during mitosis. The kinases TTK/hMps1, Bub1, and BubR1 are involved in this so-called spindle checkpoint (summarizing overview in: Kops et al. On the road to cancer: aneuploidy and the mitotic checkpoint. Nat Rev Cancer. 2005 October; 5(10):773-85). These are localized on kinetochores of condensed chromosomes which are not yet attached to the spindle apparatus and inhibit the so-called anaphase-promoting complex/cyclosome (APC/C). Only after complete and correct attachment of the spindle apparatus to the kinetochores are the spindle checkpoint kinases Mps-1, Bub1, and BubR1 inactivated, thus activating APC/C and resulting in separation of the paired chromosomes. Inhibition of the spindle checkpoint kinases leads to separation of the paired chromosomes before all the kinetochores are attached to the spindle apparatus, and consequently to faulty chromosome distributions which are not tolerated by cells and finally lead to cell cycle arrest or cell death.

F. Anti-Apoptotic Kinases

Various mechanisms protect a cell from cell death during non-optimal living conditions. In tumour cells, these mechanisms lead to a survival advantage of the cells in the growing mass of the tumour, which is characterized by deficiency of oxygen, glucose and further nutrients, make it possible for tumour cells to survive without attachment to the extracellular matrix, possibly leading to metastasis, or lead to resistances to therapeutic agents. Essential anti-apoptotic signalling pathways include the PDK1-AKT/PKB signalling pathway (Altomare & Testa. Perturbations of the AKT signaling pathway in human cancer. Oncogene. 24, 7455, 2005), the NFkappaB signalling pathway (Viatour et al. Phosphorylation of NFkB and IkB proteins: implications in cancer and inflammation), the PIM1 signalling pathway (Hammerman et al. Pim and Akt oncogenes are independent regulators of hematopoietic cell growth and survival. Blood. 2005 105, 4477, 2005) and the integrin-linked kinase (ILK) signalling pathway (Persad & Dedhar. The role of integrin-linked kinase (ILK) in cancer progression. Cancer Met. Rev. 22, 375, 2003). Inhibition of the anti-apoptotic kinases such as, for example, AKT/PBK, PDK1, IkappaB kinase (IKK), PIM1, or ILK sensitizes the tumour cells to the effect of therapeutic agents or to unfavourable living conditions in the tumour environment. After inhibition of the anti-apoptotic kinases, tumour cells will react more sensitively to disturbances of mitosis caused by Aurora inhibition and undergo cell death in increased numbers.

G. Migratory Kinases

A precondition for invasive, tissue-infiltrating tumour growth and metastasis is that the tumour cells are able to leave the tissue structure through migration. Various cellular mechanisms are involved in regulating cell migration: integrin-mediated adhesion to proteins of the extracellular matrix regulates via the activity of focal adhesion kinase (FAK); control of the assembling of contractile actin filaments via the RhoA/Rho kinase (ROCK) signalling pathway (summarizing overview in M. C. Frame, Newest findings on the oldest oncogene; how activated src does it. J. Cell Sci. 117, 989, 2004).

The compounds according to the invention are effective for example
against cancer such as solid tumours, tumour growth or metastasis growth, especially:
ataxia-telangiectasia, basal cell carcinoma, bladder carcinoma, brain tumour, breast cancer, cervical carcinoma, tumours of the central nervous system, colorectal carcinoma, endometrial carcinoma, stomach carcinoma, gastrointestinal carcinoma, head and neck tumours, acute lymphocytic leukaemia, acute myelogenous leukaemia, chronic lymphocytic leukaemia, chronic myelogenous leukaemia, hairy cell leukaemia, liver carcinoma, lung tumour, non-small-cell lung carcinoma, small-cell lung carcinoma, B-cell lymphoma, Hodgkin's lymphoma, non-Hodgkin's lymphoma, T-cell lymphoma, melanoma, mesothelioma, myeloma, myoma, tumours of the oesophagus, oral tumours, ovarian carcinoma, pancreatic tumours, prostate tumours, renal carcinoma, sarcoma, Kaposi's sarcoma, leiomyosarcoma, skin cancer, squamous cell carcinoma, testicular cancer, thyroid cancer, connective tissue tumour of the gastrointestinal tissue, connective tissue sarcoma of the skin, hypereosinophilic syndrome, mast cell cancer,
for cardiovascular disorders such as stenoses, arterioscleroses and restenoses, stent-induced restenosis,
for angiofibroma, Crohn's disease, endometriosis, haemangioma.

Formulation of the compounds according to the invention to give pharmaceutical products takes place in a manner known per se by converting the active ingredient(s) with the excipients customary in pharmaceutical technology into the desired administration form.

Excipients which can be employed in this connection are, for example, carrier substances, fillers, disintegrants, binders, humectants, lubricants, absorbents and adsorbents, diluents, solvents, cosolvents, emulsifiers, solubilizers, masking flavours, colorants, preservatives, stabilizers, wetting agents, salts to alter the osmotic pressure or buffers.

Reference should be made in this connection to Remington's Pharmaceutical Science, 15th ed. Mack Publishing Company, East Pennsylvania (1980).

The pharmaceutical formulations may be

in solid form, for example as tablets, coated tablets, pills, suppositories, capsules, transdermal systems or

in semisolid form, for example as ointments, creams, gels, suppositories, emulsions or

in liquid form, for example as solutions, tinctures, suspensions or emulsions.

Excipients in the context of the invention may be, for example, salts, saccharides (mono-, di-, tri-, oligo- and/or polysaccharides), proteins, amino acids, peptides, fats, waxes, oils, hydrocarbons and their derivatives, where the excipients may be of natural origin or may be obtained by synthesis or partial synthesis.

Suitable for oral or peroral administration are in particular tablets, coated tablets, capsules, pills, powders, granules, pastilles, suspensions, emulsions or solutions.

Suitable for parenteral administration are in particular suspensions, emulsions and especially solutions.

Preparation of the Compounds According to the Invention

Sulphoximines generally have high stability in relation to structure and configuration (C. Bolm, J. P. Hildebrand, J. Org. Chem. 2000, 65, 169). These properties of the functional group frequently even allow drastic reaction conditions and enable simple derivatization of the sulphoximines on the imine nitrogen and the α carbon. Enantiopure sulphoximines are also used as auxiliaries in diastereoselective synthesis ((a) S. G. Pyne, Sulphur Reports 1992, 12, 57; (b) C. R. Johnson, Aldrichchimica Acta 1985, 18, 3). The preparation of enantiopure sulphoximines is described for example by racemate resolution with enantiopure camphor-10-sulphonic acid ((a) C. R. Johnson, C. W. Schroeck, J. Am. Chem. Soc. 1973, 95, 7418; (b) C. S. Shiner, A. H. Berks, J. Org. Chem. 1988, 53, 5543). A further method for preparing optically active sulphoximines consists of stereoselective imination of optically active sulphoxides ((a) C. Bolm, P. Müller, K. Harms, Acta Chem. Scand. 1996, 50, 305; (b) Y. Tamura, J. Minamikawa, K. Sumoto, S. Fujii, M. Ikeda, J. Org. Chem. 1973, 38, 1239; (c) H. Okamura, C. Bolm, Organic Letters 2004, 6, 1305).

Process Variant 1

The compounds according to the invention can be prepared by a process which is characterized by the following steps:
a) functionalization of position 4 of 2,4-dichloropyrimidine derivatives of the formula 1a by reaction with nucleophiles under basic conditions, where appropriate with use of a protective group for group X, which is eliminated again where appropriate after introduction of 1b into position 4 of 1a, 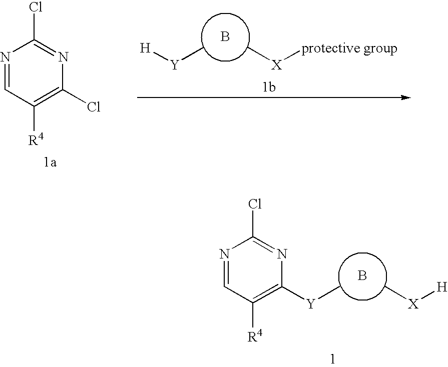
b) oxidation of a compound of the formula 2a to the sulphoxide of the formula 2b 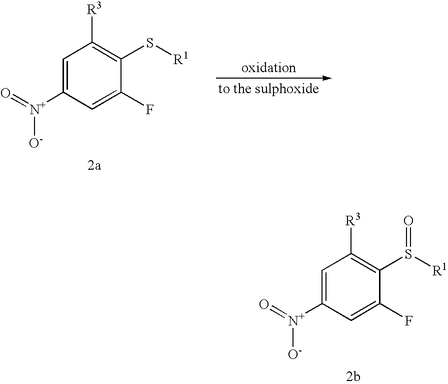
c₁) reaction of the compound of the formula 2b with sodium azide/sulphuric acid to give a compound of the formula 2c and N-functionalization of the sulphoximine to give a compound of the formula 2 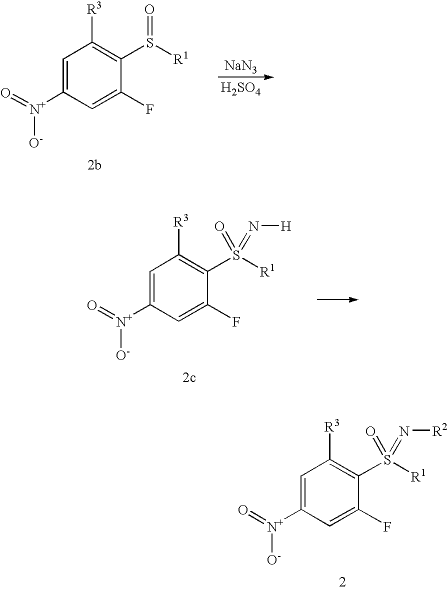
   or
c₂) direct reaction of the sulphoxide of the formula 2b to give a compound of the formula 2, 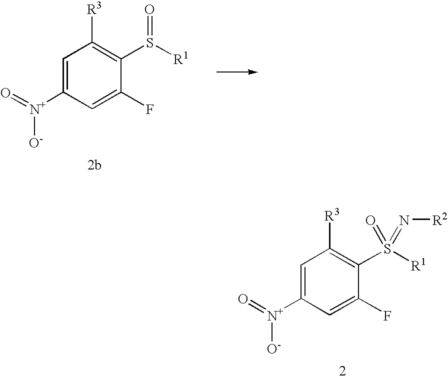
d) reaction of the compound of the formula 1 from process step a) with the compound of the formula 2 from process step c₁) or c₂) by a nucleophilic aromatic substitution to give a compound of the formula 3 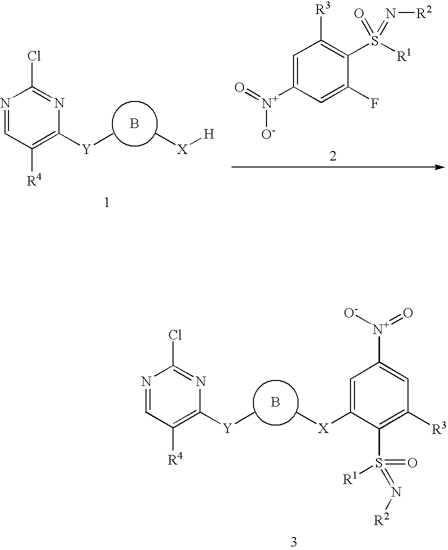
e) reduction of the compound of the formula 3 to a compound of the formula 4 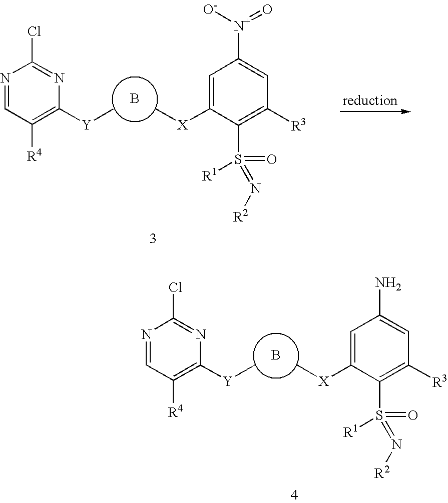
f) cyclization of the compound of the formula 4 under acidic or neutral conditions to give a compound of the formula I 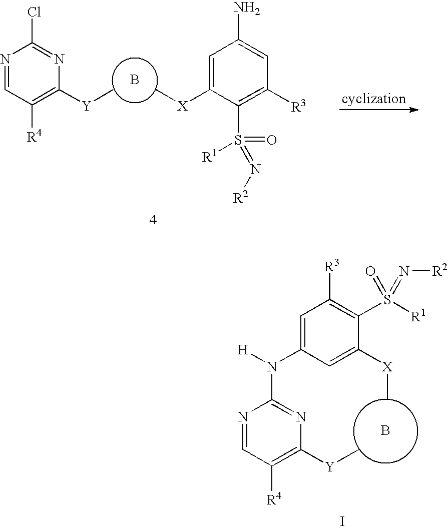
   Process Step a)

2,4-Dichloropyrimidine derivatives of the formula 1a can be functionalized in position 4 by reaction with nucleophiles under basic conditions (see, for example: a) U. Lücking, M. Krüger, R. Jautelat, G. Siemeister, WO 2005037800; b) U. Lücking, M. Krueger, R. Jautelat, O. Prien, G. Siemeister, A. Ernst, WO 2003076437; c) T. Brumby, R. Jautelat, O. Prien, M. Schäfer, G. Siemeister, U. Lücking, C. Huwe, WO 2002096888).

For N nucleophiles (Y═NH) in particular acetonitrile is suitable as solvent and triethylamine as base. The reaction preferably takes place at room temperature. For O nucleophiles (Y═O) in particular THF of DMF is suitable as solvent and sodium hydride as base. The reaction preferably takes place at 0° C. to room temperature.

For S nucleophiles (Y═S) in particular acetonitrile is suitable as solvent and triethylamine as base. The reaction preferably takes place at −20° C. to room temperature.

Depending on the nature of the substitutents X and Y, the use of a suitable protective group (PG) for group X is necessary where appropriate. The protective group (PG) is eliminated again where appropriate after successful introduction of 1b into position 4 of 1a (see, for example, T. W. Greene, P. G. M. Wuts, Protective Groups in Organic Synthesis, 2nd Edition, John Wiley & Sons, 1991).

Process Steps b)

A compound of the formula 2a is initially oxidized to the sulphoxide of the formula 2b. Numerous methods are available for conversion of a thioether into a sulphoxide (see, for example: a) M. H. Ali, W. C. Stevens, Synthesis 1997, 764-768; b) I. Fernandez, N. Khiar, Chem. Rev. 2003, 103, 3651-3705). The described used of periodic acid/iron(III) chloride is particularly suitable for preparing compounds of the formula 2b.

Process Step c₁)

A compound of the formula 2b can be reacted to give a compound of the formula 2c for example by use of sodium azide/sulphuric acid (see also: M. Reggelin, C. Zur, Synthesis 2000, 1, 1). The use of fuming sulphuric acid (oleum) is particularly suitable.

Various methods are available for further N-functionalization of the sulphoximine 2c to form compounds of the formula 2:
Alkylation (see, for example: C. R. Johnson, J. Org. Chem. 1993, 58, 1922-1923).
Acylation (see, for example: a) C. P. R. Hackenberger, G. Raabe, C. Bolm, Chem. Europ. J. 2004, 10, 2942-2952; b) C. Bolm, C. P. R. Hackenberger, O. Simic, M. Verrucci, D. Müller, F. Bienewald, Synthesis 2002, 7, 879-887; c) C. Bolm, G. Moll, J. D. Kahmann, Chem. Europ. J. 2001, 7, 1118-1128).
Arylation (see, for example: a) C. Bolm, J. P. Hildebrand, Tetrahedron Lett. 1998, 39, 5731-5734; b) C. Bolm, J. P. Hildebrand, J. Org. Chem. 2000, 65, 169-175; c) C. Bolm, J. P. Hildebrand, J. Rudolph, Synthesis 2000, 7, 911-913; d) Y. C. Gae, H. Okamura, C. Bolm, J. Org. Chem. 2005, 70, 2346-2349).
Reaction with isocyanates/isothiocyanates (see, for example: a) V. J. Bauer, W. J. Fanshawe, S. R. Safir, J. Org. Chem. 1966, 31, 3440-3441; b) C. R. Johnson, M. Haake, C. W. Schroeck, J. Am. Chem. Soc. 1970, 92, 6594-6598; c) S. Allenmark, L. Nielsen, W. H. Pirkle, Acta Chem. Scand. Ser. B 1983, 325-328)
Reaction with sulphonyl chlorides (see, for example: a) D. J. Cram, J. Day, D. R. Rayner, D. M von Schriltz, D. J. Duchamp, D. C. Garwood. J. Am. Chem. Soc. 1970, 92, 7369-7384), b) C. R. Johnson, H. G. Corkins, J. Org. Chem. 1978, 43, 4136-4140; c) D. Craig, N. J. Geach, C. J. Pearson, A. M. Z. Slawin, A. J. P. White, D. J. Williams, Tetrahedron 1995, 51, 6071-6098).
Reaction with chloroformates or anhydrides (see, for example: a) D. J. Cram, J. Day, D. R. Rayner, D. M von Schriltz, D. J. Duchamp, D. C. Garwood. J. Am. Chem. Soc. 1970, 92, 7369-7384), b) S. G. Pyne, Z. Dong, B. W. Skelton, A. H. Allan, J. Chem. Soc. Chem. Commun. 1994, 6, 751-752; c) C. R. Johnson, H. G. Corkins, J. Org. Chem. 1978, 43, 4136-4140; d) Y. C. Gae, H. Okamura, C. Bolm, J. Org. Chem. 2005, 2346-2349).
Silylation: (see, for example: A. J. Pearson, S. L. Blystone, H. Nar, A. A. Pinkerton, B. A. Roden, J. Yoon, J. Am. Chem. Soc. 1989, 111, 134-144).
   Process Step c₂)

A further possibility of synthesizing N-functionalized compounds of the formula 2 is direct reaction of a sulphoxide of the formula 2b, for example using the following reagents/methods:
TsN₃ ((a) R. Tanaka, K. Yamabe, J. Chem. Soc. Chem. Commun. 1983, 329; (b) H. Kwart, A. A. Kahn, J. Am. Chem. Soc. 1967, 89, 1959))
N-Tosyliminophenyliodinane and cat. amounts of Cu(1) triflate (J. F. K. Müller, P. Vogt, Tetrahedron Lett. 1998, 39, 4805)
Boc-Azide and cat. amounts of iron(II) chloride (T. Bach, C. Korber, Tetrahedron Lett. 1998, 39, 5015) or
o-Mesitylenesulphonylhydroxylamine (MSH) (C. R. Johnson, R. A. Kirchhoff, H. G. Corkins, J. Org. Chem. 1974, 39, 2458)
[N-(2-(Trimethylsilyl)ethanesulphonyl)imino]phenyliodinane (PhI=NSes) (S. Cren, T. C. Kinahan, C. L. Skinner and H. Tye, Tetrahedron Lett. 2002, 43, 2749).
Trifluoracetamide or sulphonylamides in combination with iodobenzene diacetate, magnesium oxide and catalytic amounts of rhodium(II) acetate dimer (H. Okamura, C. Bolm, Organic Letters 2004, 6, 1305.
Sulphonylamides in combination with iodobenzene diacetate and catalytic amounts of a chelating ligand and silver salts (G. Y. Cho, C. Bolm, Organic Letters 2005, 7, 4983).
NsNH₂ and iodobenzene diacetate (G. Y. Cho, C. Bolm, Tetrahedron Lett. 2005, 46, 8007).
   Process Step d)

In process variant 1, initially the compounds of the formula 1 and of the formula 2 are reacted by a nucleophilic aromatic substitution (see, for example: a) F. A. Carey, R. J. Sundberg, Organische Chemie, VCH, Weinheim, 1995, 1341-1359; b) Organikum, VEB Deutscher Verlag der Wissenschaften, Berlin, 1976, 421-430) to give a compound of the formula 3. Particularly suitable in this connection are polar aprotic solvents such as, for example, DMF or DMSO. The bases to be used may be varied depending on the nature of the nucleophile: for X═NH for example triethylamine is suitable, for X═O for example NaH is suitable and for X═S it is possible to use for example NaH, triethylamine or potassium carbonate.

Process Step e)

A number of reaction conditions are available in principle for the subsequent reduction of the aromatic nitro group to a compound of the formula 4 (see, for example: R. C. Larock, Comprehensive Organic Transformations, VCH, New York, 1989, 411-415). The described used of titanium(III) chloride is particularly suitable.

Process Step f)

The compound of the formula 4 is finally cyclized in the presence of an acid such as, for example, hydrogen chloride, or under neutral conditions to give a compound of the formula I. Various solvents/solvent mixtures can be used depending on the nature of the compound of the formula 4. It is particularly suitable for example to use acetonitrile or acetonitrile/water. It is further possible to use acidic, aqueous solutions or else water as solvent. The reaction temperature may be varied depending on the reactivity of the compound of the formula 4 and of the acid used and of the solvent used in the range from room temperature to reflux. The temperature range of 60-90° C. is particularly suitable for acetonitrile and acetonitrile/water mixtures in combination with hydrogen chloride as acid. Moreover, the cyclization in a microwave at relatively high temperatures and with relatively short reaction times is also very suitable. The use of HCl/water or water as solvent is particularly suitable for the reaction in a microwave. The reactions are preferably carried out in the temperature range of 110-160° C.

Process Variant 2

The compounds according to the invention in which X is —O— can be prepared by a process which is characterized by the following steps:
a) reaction of an alcohol of the formula 6 with a phenol of the formula 7 under Mitsunobu conditions 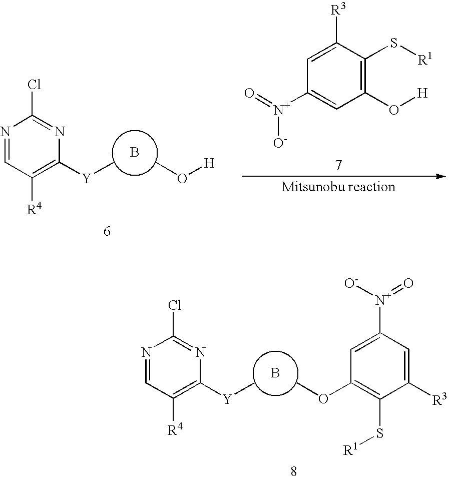
b₁) (i) oxidation of the thioether of the formula 8 to the sulphoxide and subsequently

(ii) reaction to give the sulphoximine of the formula 9. 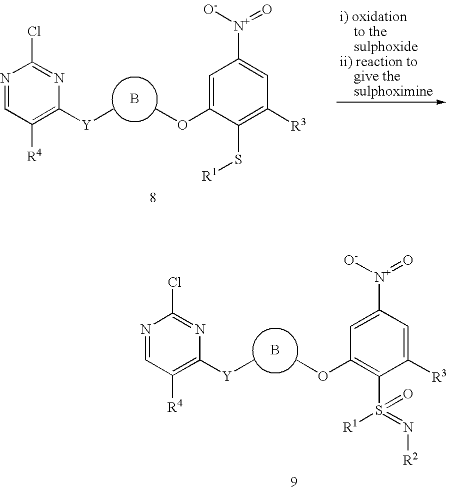
b₂) In the case of compounds of the type 9, in which R²═H, an N-functionalization of the sulphoximine can take place at this stage. 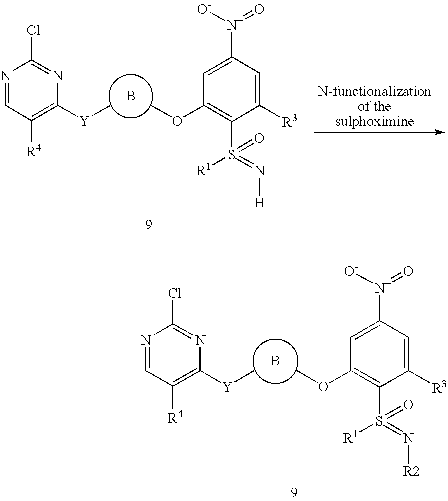
c₁) (i) reduction of the compound of the formula 9 and
   (ii) cyclization under acidic or neutral conditions to give compounds of the formula II. 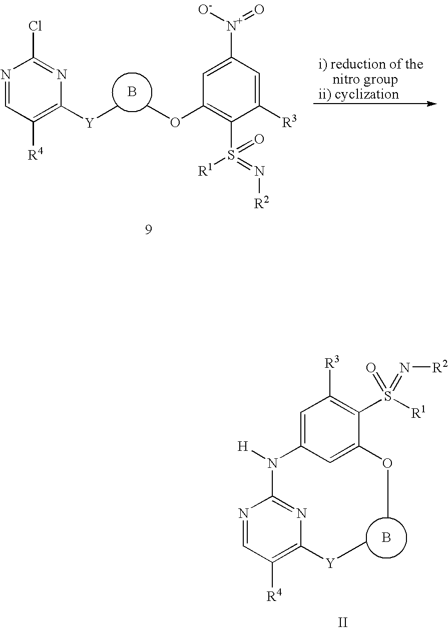
c₂) In the case of compounds of the formula II, in which R2=H, an N-functionalization of the sulphoximine can take place. 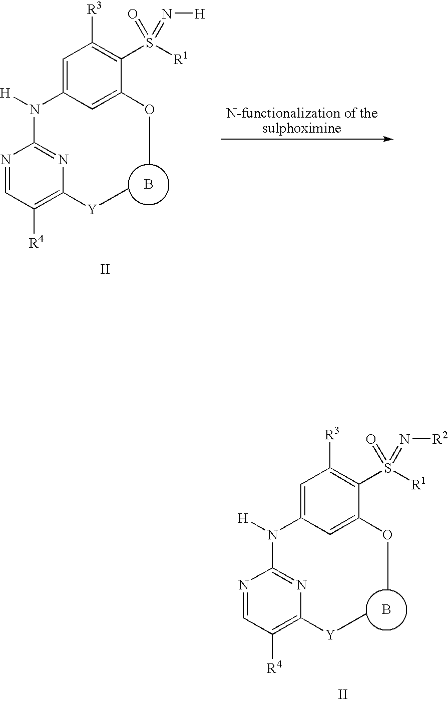
   Process Step a)

In process variant 2, alcohols of the formula 6 are coupled with phenols of the formula 7 under Mitsunobu conditions (see, for example: a) O. Mitsunobu, M. Yamada, T. Mukaiyama, Bull. Chem. Soc. Jpn. 1967, 40, 935; b) O. Mitsunobu, Synthesis 1981, 1; c) D. L. Hughes, ‘The Mitsunobu Reaction’, Organic Reactions, John Wiley & Sons, Ltd, 1992, 42, 335) to give compounds of the formula 8.

Process Step b₁)

(i) Firstly the thioether of the formula 8 is oxidized to the sulphoxide. Numerous methods are available for converting a thioether into a sulphoxide (see, for example: a) M. H. Ali, W. C. Stevens, Synthesis 1997, 764-768; b) I. Fernandez, N. Khiar, Chem. Rev. 2003, 103, 3651-3705). The described use of periodic acid/iron(III) chloride for example is particularly suitable.
(ii) Reaction to give the sulphoximine of the formula 9 subsequently takes place. Preferred methods here are for example reaction of the sulphoxide with [N-(2-(trimethylsilyl)ethanesulphonyl)imino]phenyliodinane (PhI=NSes) (S. Cren, T. C. Kinahan, C. L. Skinner and H. Tye, Tetrahedron Lett. 2002, 43, 2749) or trifluoroacetamide or sulphonylamides in combination with iodobenzene diacetate, magnesium oxide and catalytic amounts of rhodium(II) acetate dimer (H. Okamura, C. Bolm, Organic Letters 2004, 6, 1305). The described use of fuming sulphuric acid (oleum) and sodium azide is particularly preferred.
   Process Step b₂)

In the case of compounds of the type 9, in which R2=H, an N-functionalization of the sulphoximine can take place by the methods mentioned under process variant 1, process step c₁).

Process Step c₁)

(i) A number of reaction conditions are available in principle for the subsequent reduction of the aromatic nitro group (see, for example: R. C. Larock, Comprehensive Organic Transformations, VCH, New York, 1989, 411-415). The described use of titanium(III) chloride is particularly suitable.
(ii) Subsequently, cyclization takes place under acidic or neutral conditions to give compounds of the formula II. Cyclization in a microwave at relatively high temperatures and with relatively short reaction times is particularly preferred. The use of HCl/water or water as solvent is particularly suitable for the reaction in a microwave. The reactions are preferably carried out in the temperature range of 110-160° C.
   Process Step c₂)

In the case of compounds of the formula II, in which R2=H, an N-functionalization of the sulphoximine can take place by the methods mentioned under process variant 1, process step c₁).
Process Variant 3:

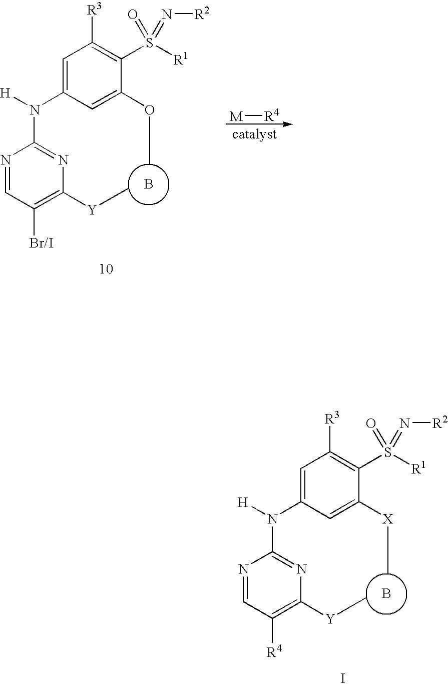

In process variant 3,5-bromo or 5-iodo derivatives of the formula 10 are reacted
in a Suzuki coupling (see, for example: a) F. Bellina, A. Carpita, R. Rossi. Synthesis 2004, 15, 2419; b) V. Wittmann, Nachrichten aus der Chemie 2002, 50, 1122; c) A. Herrmann, Applied Homogeneous Catalysis with Organometallic Compounds (2nd Edition) 2002, 1, 591; d) A. Suzuki in F. Diederich, P. J. Stang (Eds.) Metal-catalyzed Cross-Coupling Reactions, Wiley-VCH, New York, 1998, 47) with boronic acid derivatives (M=B(OH)₂ or B(OR)₂) or
in a Stille coupling (see, for example: a) Oliver Reiser, Chemie in unserer Zeit 2001, 35, 94; b) V. Farina, V. Krishnamurthy, W. J. Scott, Org. React. (N.Y.) 1997, 50, 1) with tin derivatives (M=SnR₃) or
in a Negishi coupling (see, for example: a) E. Negishi, X. Zeng,; Metal-Catalyzed Cross-Coupling Reactions (2nd Edition) 2004, 2, 815; b) E. Negishi Handbook of Organopalladium Chemistry for Organic Synthesis 2002, 1, 229-) with zinc derivatives (M=ZnR).

The following examples serve to explain the invention in more detail without restricting the invention thereto.

Process Variant 1

### EXAMPLE 1

### (RS)—S-[1⁵-Bromo-2,4,9-triaza-1(2,4)-pyrimidina-3(1,3)-benzenacyclononaphan-3⁴-yl]-N-(ethoxycarbonyl)-S-methylsulphoximide

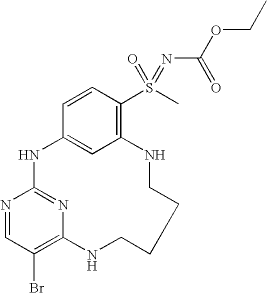 1. Preparation of the Intermediates
a) Compound 1.1:

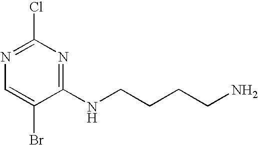

6.84 ml (49.4 mmol) of triethylamine are added to a suspension of 5.37 g (23.6 mmol) of 5-bromo-2,4-dichloropyrimidine and 4.88 g (25.9 mmol) of N-Boc-1,4-diaminobutane in 102 ml of acetonitrile while cooling in water. The reaction mixture is stirred at room temperature overnight and then added to NaCl solution. The mixture is extracted with ethyl acetate (3×). The combined organic phases are dried (Na₂SO₄), filtered and concentrated. The resulting residue is mixed with 250 ml of acetonitrile and 45 ml of a 4 N solution of hydrogen chloride in dioxane and stirred at room temperature for 2 hours. The mixture is evaporated to dryness. Toluene is added and the mixture is again evaporated to dryness. 8.50 g of the crude product are obtained as hydrochloride which is employed without further purification.
b) Compound 1.2

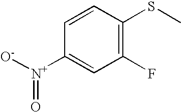

13.8 g (196.6 mmol) of sodium methanethiolate are added in portions to an ice-cooled solution of 25.7 g (161.5 mmol) of 1,2-difluoro-4-nitrobenzene in 172 ml of DMF and the mixture is stirred at room temperature for 24 hours. The mixture is added to ice-water and extracted with ethyl acetate (3×). The combined organic phases are dried (Na₂SO₄), filtered and concentrated. The resulting crude product is employed without further purification.

¹H-NMR (DMSO): 8.02 (m, 2H), 7.48 (m, 1H), 2.57 (s, 3H).
c) Compound 1.3

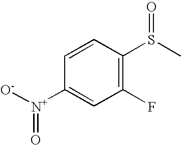

33.8 g (148 mmol) of periodic acid are added to a mixture of 25.9 g (138.5 mmol) of compound 1.2 and 644 mg (4.0 mmol) of iron(III) chloride in 112 ml of acetonitrile at room temperature. The reaction temperature is kept below 30° C. by cooling in water. The suspension is stirred at RT for 1 hour and then added to a mixture of 250 ml of DCM, 750 ml of ice-water and 150 g of sodium thiosulphate pentahydrate. The mixture is extracted with DCM (3×). The combined organic phases are dried (Na₂SO₄), filtered and concentrated. The resulting crude product is recrystallized from ethyl acetate/hexane. 15.7 g (77.2 mmol; corresponding to 56% of theory) of the product are obtained.

¹H-NMR (DMSO): 8.35 (m, 2H), 8.00 (m, 1H), 2.91 (s, 3H).
d) Compound 1.4

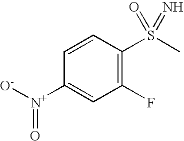

20.6 ml of conc. sulphuric acid are added dropwise to an ice-cooled suspension of 15.7 g (77.2 mmol) of compound 1.3 and 10.0 g (154.3 mmol) of sodium azide in 27 ml of chloroform while stirring. The mixture is slowly warmed to 45° C. and then stirred at this temperature for 24 hours. After cooling, the mixture is added to 800 ml of ice-water and basified with solid NaOH. Saturation with solid NaCl is followed by extraction with DCM (3×). The combined organic phases are washed with saturated NaCl solution (2×), dried (Na₂SO₄), filtered and concentrated. 15.3 g of the crude product are obtained and employed without further purification.

¹H-NMR (DMSO): 8.35 (m, 1H), 8.24 (m, 1H), 8.08 (m, 1H), 5.07 (s, 1H), 3.22 (s, 3H).
e) Compound 1.5

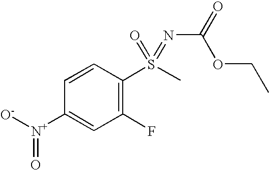

10.2 ml (106.4 mmol) of ethyl chloroformate are added dropwise to an ice-cooled solution of 5.0 g (22.9 mmol) of compound 1.4 in 215 ml of pyridine while stirring. The mixture is warmed to room temperature overnight and then added to saturated NaCl solution. It is extracted with ethyl acetate (3×). The combined organic phases are dried (Na₂SO₄), filtered and concentrated. 6.54 g of the crude product are obtained and employed without further purification.

¹H-NMR (DMSO): 8.43 (m, 1H), 8.29 (m, 1H), 8.15 (m, 1H), 3.87 (m, 2H), 3.55 (s, 3H), 1.05 (tr, 3H).
f) Compound 1.6

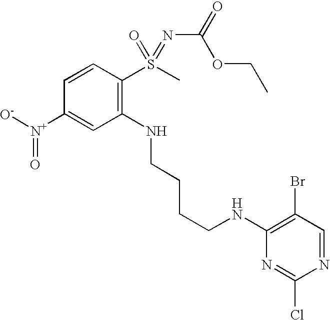

2.2 ml (15.6 mmol) of triethylamine are added to a suspension of 1.50 g (5.2 mmol) of compound 1.5 and 2.45 g (7.8 mmol) of compound 1.1 in 15 ml of acetonitrile under argon and stirred at room temperature for 5 min. The mixture is then warmed to 60° C. and stirred at this temperature for 8 hours. After cooling, the mixture is added to saturated NaCl solution and extracted 3× with ethyl acetate. The combined organic phases are dried (Na₂SO₄), filtered and concentrated. The remaining residue is purified by chromatography (hexane/ethyl acetate 1:1). 2.03 g (3.7 mmol; corresponding to 71% of theory) of the product are obtained.

¹H-NMR (DMSO): 8.19 (s, 1H), 7.86 (m, 1H), 7.73 (tr, 1H), 7.46 (m, 2H), 6.54 (tr, 1H), 3.86 (m, 2H), 3.44 (s, 3H), 3.35 (m, 4H), 1.60 (m, 4H), 1.04 (tr, 3H).
g) Compound 1.7:

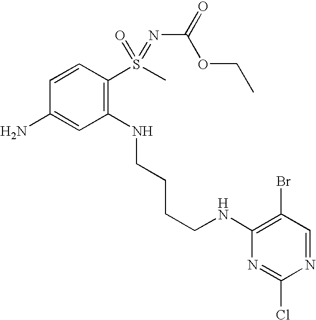

19 ml of an approx. 10% strength solution of titanium(III) chloride in 20-30% strength hydrochloric acid are added over a period of 20 minutes to a solution of 1.0 g (1.82 mmol) of compound 1.6 in 40 ml of THF under argon at 0° C. The mixture is slowly warmed to room temperature. After 4 hours, the mixture is cooled in an ice bath and adjusted to pH 7-8 with 1N NaOH solution. It is extracted with ethyl acetate (3×). The combined organic phases are dried (Na₂SO₄), filtered and concentrated. The remaining residue is purified by chromatography (DCM/EtOH 9:1). 736 mg (1.42 mmol, corresponding to 78% of theory) of the product are obtained.

¹H-NMR (DMSO): 8.19 (s, 1H), 7.73 (tr, 1H), 7.21 (m, 1H), 6.07 (tr, 1H), 5.88 (m, 4H), 3.88 (q, 2H), 3.38 (m, 2H), 3.20 (s, 3H), 3.03 (m, 2H), 1.60 (m, 4H), 1.06 (tr, 3H).

2. Preparation of the Final Product

A solution of 557 mg (1.07 mmol) of compound 1.7 in acetonitrile/water/methanol (35 ml/3.5 ml/3.5 ml) is added by means of a syringe driver over the course of 3 hours to a solution of acetonitrile/water/4 N solution of hydrogen chloride in dioxane (156 ml/17 ml/1.7 ml) at 60° C. After 68 hours, the mixture is evaporated and the resulting residue is purified by chromatography (DCM/EtOH 9:1). 223 mg (0.46 mmol, corresponding to 43% of theory) of the product are obtained.

¹H-NMR (DMSO): 9.80 (s, 1H), 8.61 (br, 1H), 8.08 (s, 1H), 7.74 (br, 1H), 7.37 (m, 1H), 6.47 (m, 1H), 6.38 (br, 1H), 3.88 (q, 2H), 3.38 (m, 4H), 3.27 (s, 3H), 1.78 (m, 2H), 1.63 (m, 2H), 1.06 (tr, 3H).

MS: 483 (ES+).

### EXAMPLE 2

The racemate from Example 1 is separated into the enantiomers by preparative chiral HPLC:

Column: Chiralpak AD-H 5μ; 250×20 mm

Eluent: hexane/ethanol; isocratic 50% ethanol

Flow rate: 10.0 ml/min

Detector: UV 300 nM

Temperature: RT

Retention: enantiomer 1: 24.94 min

### EXAMPLE 3

The racemate from Example 1 is separated into the enantiomers by preparative chiral HPLC:

Column: Chiralpak AD-H 5μ; 250×20 mm

Eluent: hexane/ethanol; isocratic 50% ethanol

Flow rate: 10.0 ml/min

Detector: UV 300 nM

Temperature: RT

Retention: enantiomer 2: 38.69 min

### EXAMPLE 4

### (RS)—N-(Ethoxycarbonyl)-S-[1⁵-iodo-2,4,9-triaza-1(2,4)-pyrimidina-3(1,3)-benzenacyclononaphan-3⁴-yl]-S-methylsulphoximide

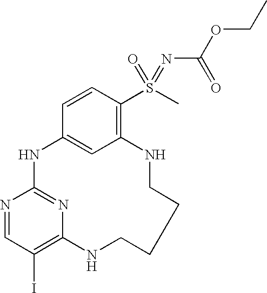 1. Preparation of the Intermediates:
a) Compound 4.1:

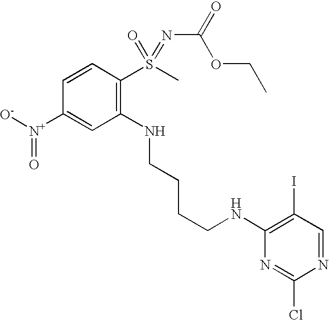

Reaction of 1.20 g (4.10 mmol) of compound 1.5 with 1.75 g (4.8 mmol) of N-(2-chloro-5-iodopyrimidin-4-yl)propane-1,3-diamine hydrochloride by the method for preparing compound 1.6 results in the product in 98% yield (2.40 g; 4.02 mmol)).

¹H-NMR (DMSO): 8.28 (s, 1H), 7.88 (m, 1H), 7.48 (m, 2H), 7.35 (tr, 1H), 6.52 (tr, 1H), 3.88 (m, 2H), 3.42 (s, 3H), 3.30 (m, 4H), 1.59 (m, 4H), 1.03 (tr, 3H).
b) Compound 4.2:

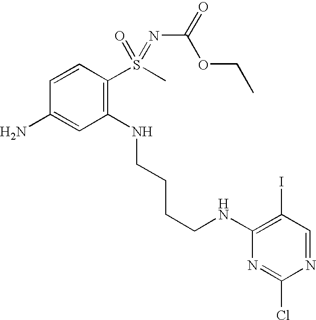

Reaction of 1.20 g (2.01 mmol) of compound 4.1 by the method for preparing compound 1.7 results in the product in 62% yield (0.70 g; 1.24 mmol).

¹H-NMR (DMSO): 8.28 (s, 1H), 7.34 (tr, 1H), 7.20 (m, 1H), 6.05 (tr, 1H), 5.88 (m, 4H), 3.87 (q, 2H), 3.35 (m, 2H), 3.19 (s, 3H), 3.03 (m, 2H), 1.53 (m, 4H), 1.06 (tr, 3H).

MS: 567 (ES+).

2. Preparation of the Final Product

A solution of 350 mg (0.61 mmol) of compound 4.2 in 10 ml of acetonitrile is added by means of a syringe driver over the course of 3 hours to a solution of acetonitrile/water/4 N solution of hydrogen chloride in dioxane (45.0 ml/5.0 ml/0.5 ml) at 60° C. After 16 hours, the mixture is evaporated and the resulting residue is purified by chromatography (DCM/EtOH 9:1). 160 mg (0.30 mmol, corresponding to 49% of theory) of the product are obtained.

¹H-NMR (DMSO): 9.53 (s, 1H), 8.69 (br, 1H), 8.12 (s, 1H), 7.33 (m, 1H), 7.08 (tr, 1H), 6.47 (m, 1H), 6.35 (tr, 1H), 3.88 (m, 2H), 3.30 (m, 7H), 1.59 (m, 4H), 1.08 (tr, 3H).

MS: 531 (ES+).

### EXAMPLE 5

### (RS)—S-[1⁵-Bromo-2,4,9-triaza-1(2,4)-pyrimidina-3(1,3)-benzenacyclononaphan-3⁴-yl]-S-methyl-N-[2-(trimethylsilyl)ethylsulphonyl]sulphoximide

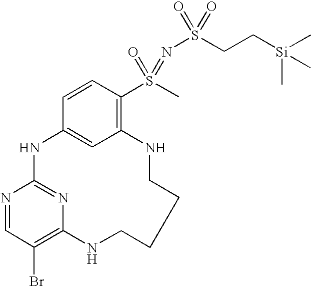 1. Preparation of the Intermediates
a) Compound 5.1

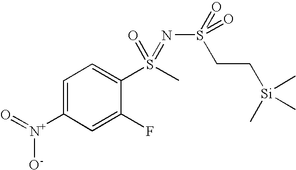

A solution of 2.12 g (10.5 mmol) of SES-CI (L. L. Parker, N. D. Gowans, S. W. Jones, D. J. Robin; Tetrahedron 2003, 59, 10165) in 25 ml of DCM is added over a period of 10 minutes to a solution of 1.90 g (8.7 mmol) of compound 1.4, 1.5 ml (10.5 mmol) of triethylamine and 106 mg (0.87 mmol) of DMAP in 25 ml of DCM while cooling in water. The mixture is stirred at room temperature for 3 hours and then mixed with dilute NaCl solution. The mixture is extracted with ethyl acetate (3×). The combined organic phases are filtered through a Whatman filter and concentrated. The resulting residue is purified by chromatography (hexane/ethyl acetate 1:1). 1.70 g (4.5 mmol; corresponding to 51% of theory) of the product are obtained.

¹H-NMR (DMSO): 8.51 (m, 1H), 8.30 (m, 1H), 8.16 (m, 1H), 3.71 (s, 3H), 2.95 (m, 2H), 0.91 (m, 2H), 0.01 (s, 9H).
b) Compound 5.2:

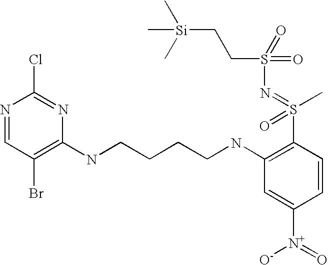

Reaction of 1.50 g (4.10 mmol) of compound 5.1 with 1.86 g (5.88 mmol) of compound 1.1 by the method for preparing compound 1.6 results in the product in 48% yield (1.21 g; 4.02 mmol)).

¹H-NMR (DMSO): 8.19 (s, 1H), 7.88 (m, 1H), 7.68 (tr, 1H), 7.49 (m, 2H), 6.49 (tr, 1H), 3.58 (s, 3H), 3.35 (m, 4H), 2.99 (m, 2H), 1.64 (m, 4H), 0.93 (m, 2H), −0.01 (s, 9H).

MS: 641 (ES+).
c) Compound 5.3:

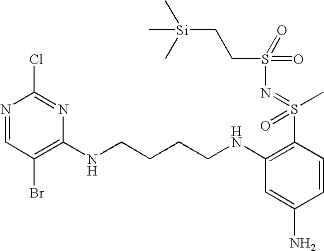

Reaction of 1.21 g (1.88 mmol) of compound 5.2 by the method for preparing compound 1.7 results in the product in 11% yield (0.13 g; 0.20 mmol).

¹H-NMR (DMSO): 8.20 (s, 1H), 7.72 (tr, 1H), 7.28 (m, 1H), 5.98 (m, 5H), 3.37 (m, 5H), 3.07 (m, 2H), 2.96 (m, 2H), 1.64 (m, 4H), 0.92 (m, 2H), −0.02 (s, 9H).

MS: 611 (ES+).

2. Preparation of the Final Product

A solution of 65 mg (0.11 mmol) of compound 5.3 in 3 ml of DCM is added by means of a syringe driver over the course of 3 hours to a solution of acetonitrile/water/4 N solution of hydrogen chloride in dioxane (45.0 ml/5.0 ml/0.5 ml) at 70° C. After 24 hours, the mixture is evaporated and the resulting residue is purified by chromatography (DCM/EtOH 9:1). 59 mg (0.10 mmol, corresponding to 96% of theory) of the product are obtained.

¹H-NMR (DMSO): 9.70 (s, 1H), 8.72 (br, 1H), 8.05 (s, 1H), 7.54 (br, 1H), 7.39 (m, 1H), 6.50 (m, 1H), 6.30 (br, 1H), 3.30 (m, 7H), 2.93 (m, 2H), 1.65 (m, 4H), 0.92 (m, 2H), −0.02 (s, 9H).

MS: 575 (ES+).

### EXAMPLE 6

### (RS)—S-[1⁵-Bromo-2,4,9-triaza-1(2,4)-pyrimidina-3(1,3)-benzenacyclononaphan-3⁴-yl]-N-(ethylcarbamoyl)-S-methylsulphoximide

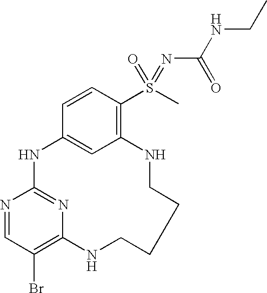 1. Preparation of the Intermediates
a) Compound 6.1:

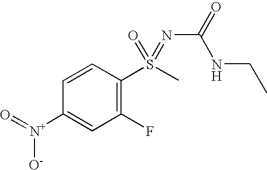

A solution of 1.32 g (6.0 mmol) of compound 1.4, 0.48 ml of ethyl isocyanate and 0.8 ml (6.0 mmol) of triethylamine in 80 ml of DCM is stirred at 40° C. for 5 days. A further 0.25 ml (3.0 mmol) of ethyl isocyanate and 0.4 ml (6.0 mmol) of triethylamine are added. After 3 days, the mixture is concentrated and the residue is purified by chromatography (DCM/EtOH 95:5). 1.30 g (4.49 mmol; corresponding to 75% of theory) of the product are obtained.

¹H-NMR (DMSO): 8.35 (m, 1H), 8.24 (m, 1H), 8.11 (m, 1H), 7.08 (tr, 1H), 3.41 (s, 3H), 2.85 (m, 2H), 0.89 (tr, 3H).

MS: 290 (ES+).
b) Compound 6.2:

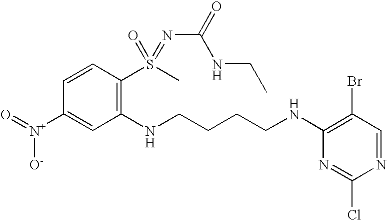

Reaction of 289 mg (1.50 mmol) of compound 6.1 with 419 mg (1.5 mmol) of compound 1.1 by the method for preparing compound 1.6, and aqueous workup with dilute citric acid, result in the crude product in quantitative yield (593 mg).

¹H-NMR (DMSO): 8.17 (s, 1H), 7.83 (m, 1H), 7.72 (tr, 1H), 7.48 (m, 2H), 7.10 (tr, 1H), 6.62 (tr, 1H), 3.35 (m, 4H), 3.30 (s, 3H), 2.91 (m, 2H), 1.61 (m, 4H), 0.92 (tr, 3H).

MS: 548 (ES+).
c) Compound 6.3:

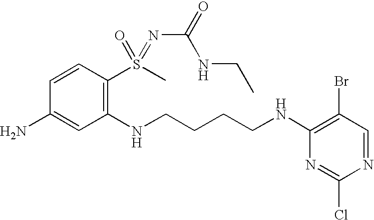

Reaction of 590 mg (1.07 mmol) of compound 6.2 by the method for preparing compound 1.7 results in the product in 39% yield (216 mg, 0.42 mmol).

¹H-NMR (DMSO): 8.19 (s, 1H), 7.73 (tr, 1H), 7.22 (m, 1H), 6.85 (tr, 1H), 6.21 (tr, 1H), 5.95 (m, 1H), 5.84 (m, 3H), 3.38 (m, 2H), 3.17 (s, 3H), 2.98 (m, 4H), 1.60 (m, 4H), 0.93 (tr, 3H).

MS: 518 (ES+).

2. Preparation of the Final Product

A solution of 117 mg (0.23 mmol) of compound 6.3 in 5 ml of acetonitrile is added by means of a syringe driver over the course of 3 hours to a solution of acetonitrile/water/4 N solution of hydrogen chloride in dioxane (45.0 ml/5.0 ml/0.5 ml) at 80° C. and stirred for a further 19 hours. Cooling is followed by addition of NaHCO₃ solution to the mixture and extraction with ethyl acetate (3×). The combined organic phases are filtered through a Whatman filter and concentrated. The resulting residue is purified by chromatography (DCM/EtOH 95:5). 44 mg (0.09 mmol; corresponding to 40% of theory) of the product are obtained.

¹H-NMR (DMSO): 9.55 (s, 1H), 8.70 (br, 1H), 8.03 (s, 1H), 7.39 (tr, 1H), 7.31 (m, 1H), 6.96 (tr, 1H), 6.59 (br, 1H), 6.44 (m, 1H), 3.35 (m, 4H), 3.19 (s, 3H), 2.92 (m, 2H), 1.65 (m, 4H), 0.93 (tr, 3H).

MS: 482 (ES+)

### EXAMPLE 7

### (RS)—S-[1⁵-Bromo-2,4,9-triaza-1(2,4)-pyrimidina-3(1,3)-benzenacyclononaphan-3⁴-yl]-S-methyl-N-(propylsulphonyl)sulphoximide

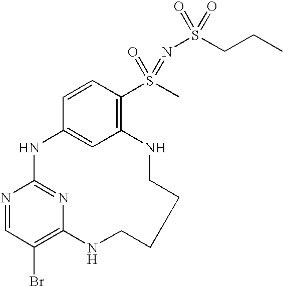 1. Preparation of the Intermediates
a) Compound 7.1

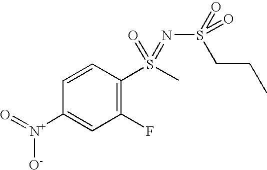

0.56 ml (5.04 mmol) of propane-1-sulphonyl chloride is added to a stirred solution of 1000 mg (4.58 mmol) of compound 1.4 in 30 ml of pyridine under argon. The mixture is stirred at room temperature for 5 hours. 0.7 ml (5.04 mmol) of triethylamine is added, and the mixture is stirred overnight. 0.52 ml (4.68 mmol) of propane-1-sulphonyl chloride is again added to the mixture, which is stirred at room temperature for a further night. Dilute citric acid is added, and the mixture is extracted with ethyl acetate (3×). The combined organic phases are washed with NaHCO₃ solution and NaCl solution, dried (Na₂SO₄), filtered and concentrated. The resulting residue is purified by chromatography (hexane/ethyl acetate 1:1). 500 mg (1.56 mmol; corresponding to 34% of theory) of the product are obtained.

MS: 325 (ES+).

b) Compound 7.2

Preparation analogous to the methods for compounds 1.6 and 1.7.

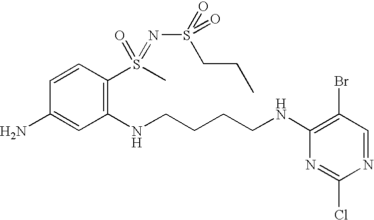

¹H-NMR (DMSO): 8.19 (s, 1H), 7.72 (tr, 1H), 7.25 (m, 1H), 5.98 (m, 3H), 5.88 (m, 2H), 3.35 (m, 5H), 3.03 (m, 4H), 1.61 (m, 6H), 0.92 (tr, 3H).

MS: 553 (ES+).

2. Preparation of the Final Product

A solution of 99 mg (0.18 mmol) of compound 7.2 in 10 ml of acetonitrile is added by means of a syringe driver over the course of 3 hours to a solution of acetonitrile/water/4 N solution of hydrogen chloride in dioxane (45.0 ml/5.0 ml/0.5 ml) at 70° C. and stirred for a further 40 hours. Cooling is followed by addition of NaHCO₃ solution to the mixture and extraction with ethyl acetate (3×). The combined organic phases are dried (Na₂SO₄), filtered and concentrated. The resulting residue is purified by chromatography (DCM/EtOH 95:5). 41 mg (0.08 mmol; corresponding to 44% of theory) of the product are obtained.

¹H-NMR (DMSO): 9.67 (s, 1H), 8.80 (br, 1H), 8.09 (s, 1H), 7.45 (m, 2H), 6.54 (m, 1H), 6.33 (tr, 1H), 3.45 (m, 7H), 3.07 (m, 2H), 1.72 (m, 6H), 0.98 (tr, 3H).

MS: 517 (ES+).

### EXAMPLE 8

### (RS)—S-[1⁵-Bromo-4-oxa-2,9-diaza-1(2,4)-pyrimidina-3(1,3)-benzenacyclononaphan-3⁴-yl]-S-methyl-N-(propoxycarbonyl)sulphoximide

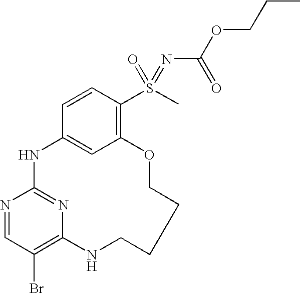 1. Preparation of the Intermediates
a) Compound 8.1:

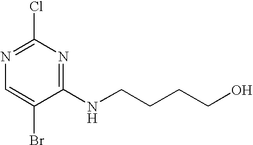

1.1 ml (12.0 mmol) of 4-aminobutanol are added to a solution of 2.28 g (10.0 mmol) of 5-bromo-2,4-dichloropyrimidine and 1.7 ml (12.0 mmol) of triethylamine in 10 ml of acetonitrile at 0° C. The reaction mixture is slowly warmed to room temperature while stirring by removing the ice bath. After 16 hours, the precipitate which has formed is filtered off. The filtrate is completely evaporated and digested with diisopropyl ether. 2.74 g (9.8 mmol, corresponding to 98% of theory) of the product are obtained.

¹H-NMR (DMSO): 8.19 (s, 1H), 7.72 (t, 1H), 4.45 (br, 1H), 3.38 (m, 4H), 1.56 (m, 2H), 1.45 (m, 2H).

MS: 279 (E1).
b) Compound 8.2:

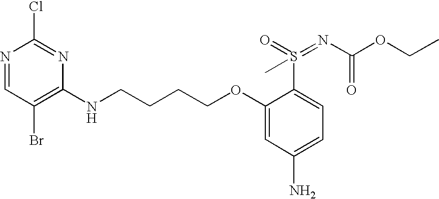

1.51 g (5.35 mmol) of compound 8.1 and 252 mg of sodium hydride (55-65%) are weighed out under argon and then 15 ml of DMF are added. The mixture is stirred at room temperature for 10 min and then a solution of 1.76 g (6.1 mmol) of compound 1.5 in 20 ml of DMF is added. The mixture is stirred overnight and added to a saturated NaCl solution. The mixture is extracted with ethyl acetate (3×). The combined organic phases are washed with saturated NaCl solution, dried (Na₂SO₄), filtered and evaporated to dryness. The resulting residue is purified by chromatography (DCM/EtOH 96:4). 1.20 g of the product, which is contaminated by a further component, are obtained. The product mixture is dissolved in 40 ml of THF under argon and, while stirring, 8.5 ml of an approx. 10% strength solution of titanium(III) chloride in 20-30% strength hydrochloric acid are added. The mixture is stirred at room temperature for 3 hours. A further 3 ml of the approx. 10% strength solution of titanium(III) chloride in 20-30% strength hydrochloric acid are added in portions over a period of 90 minutes. The mixture is diluted with ethyl acetate and then basified with NaHCO₃ solution. It is extracted with ethyl acetate (3×). The combined organic phases are dried (Na₂SO₄), filtered and concentrated. The resulting residue is purified by chromatography (DCM/EtOH 9:1). 0.30 g (0.58 mmol; corresponding to 11% of theory) of the product is obtained.

¹H-NMR (DMSO): 8.20 (s, 1H), 7.73 (tr, 1H), 7.38 (m, 1H), 6.22 (m, 2H), 6.11 (s, 2H), 3.98 (m, 2H), 3.79 (m, 2H), 3.35 (m, 2H), 3.24 (s, 3H), 1.68 (m, 4H), 1.00 (tr, 3H).

MS: 520 (ES+).

2. Preparation of the Final Product

A solution of 9 ml of propanol/1 ml of 4 N solution of hydrogen chloride in dioxane is added by means of a syringe driver over a period of 5 hours to 158 mg (0.30 mmol) of compound 8.2 in 200 ml of propanol while stirring at 70° C. The mixture is subsequently stirred at 70° C. for 40 hours and then evaporated to dryness. The resulting residue is purified by HPLC 73 mg (0.15 mmol, corresponding to 48% of theory) of the product are obtained.

¹H-NMR (DMSO): 9.91 (s, 1H), 9.08 (m, 1H), 8.07 (s, 1H), 7.57 (m, 2H), 6.81 (m, 1H), 4.41 (m, 2H), 3.72 (tr, 2H), 3.39 (m, 2H), 3.31 (s, 3H), 1.75 (m, 4H), 1.42 (m, 2H), 0.75 (tr, 3H).

HPLC:

Column: Purospher Star C18 5μ; 125×25 mm

Eluent: A: H₂O+0.1% TFA, B:MeCN;

Gradient: 76% A+24% B(1′)->24->38% B(10′)->95% B(0, 1′)

Flow rate: 25 ml/min

Detector: UV 254 nm; MS-ESI+

Temperature: RT

Retention: 8.8-9.6 min (peak: 497 m/z).

Process Variant 2

### EXAMPLE 9

### (RS)—S-[1⁵-Bromo-4-oxa-2,9-diaza-1(2,4)-pyrimidina-3(1,3)-benzenacyclononaphan-3⁴-yl]-S-methyl-N-[2-(trimethylsilyl)ethylsulphonyl]sulphoximide

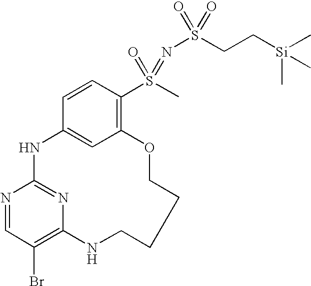 1. Preparation of the Intermediates
a) Compound 9.1

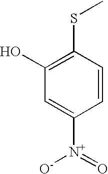

1.67 g (10.0 mmol) of 3,4-methylenedioxynitrobenzene are added to a suspension of 1.0 g (14.3 mmol) of sodium methanethiolate in 3 ml of N-methylpyrrolidone (NMP) at 35-40° C. After 30 minutes, the mixture is added to ice-water and neutralized with acetic acid. The precipitate which has formed is filtered off with suction, washed with water and dried. 1.7 g (9.1 mmol; corresponding to 91% of theory) of the product are obtained.

¹H-NMR (DMSO): 10.98 (s, 1H), 7.72 (m, 1H), 7.57 (m, 1H), 7.29 (m, 1H), 2.48 (s, 3H).
b) Compound 9.2

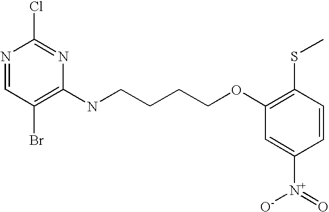

0.42 ml of DEAD reagent is added to a mixture of 606 mg (2.02 mmol) of compound 8.1, 332 mg (1.82 mmol) of compound 9.1 and 641 mg (2.44 mmol) of triphenylphosphine in 25 ml of THF at 0° C. under argon. The mixture is stirred overnight, concentrated over silica gel and purified by chromatography (hexane/ethyl acetate 4:1). 568 mg (1.27 mmol; corresponding to 63% of theory) of the product are obtained.

¹H-NMR (DMSO): 8.20 (s, 1H), 7.83 (m, 2H), 7.65 (m, 1H), 7.32 (m, 1H), 4.21 (m, 2H), 3.43 (m, 2H), 2.50 (s, 3H), 1.75 (m, 4H).
c) Compound 9.3

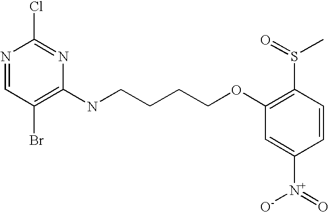

170 mg (1.05 mmol) of iron(III) chloride and 264 mg (1.15 mmol) of periodic acid are added to a mixture of 470 mg (1.05 mmol) of compound 9.2 in 30 ml of acetonitrile at room temperature. The mixture is stirred at room temperature for 20 minutes and then added to NaHCO₃ solution. The mixture is extracted with ethyl acetate (3×). The combined organic phases are washed with NaCl solution, dried (Na₂SO₄), filtered and concentrated. 468 mg of the crude product are obtained and employed without further purification.

¹H-NMR (DMSO): 8.22 (s, 1H), 8.07 (m, 1H), 7.82 (m, 3H), 4.30 (m, 2H), 3.43 (m, 2H), 2.79 (s, 3H), 1.73 (m, 4H).

MS: 463 (ES+).
d) Compound 9.4

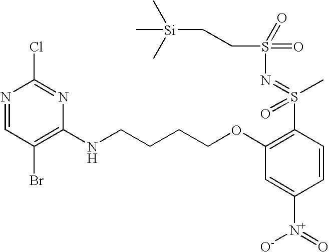

A two-neck flask with molecular sieves (3 Å) is heat-dried in vacuo and then 300 mg (0.65 mmol) of compound 9.3 are weighed in. Evacuation and flushing with argon are carried out (3×). 15 ml of acetonitrile are added, and the mixture is stirred at room temperature for 10 min. Then, under argon, 150 mg (0.40 mmol) of CuPF₆(MeCN)₄ are added and stirred at room temperature for 20 min. The reaction mixture is cooled to 0° C., and 300 mg (0.78 mmol) of Ph-I=N-Ses reagent (H. Tye, C. L. Skinner, T. C. Kinahan, S. Cren Tetrahedron Lett. 2002, 43, 2749-2751) are added. The ice bath is removed and the mixture is stirred at room temperature for 90 min. After a TLC check, the mixture is again cooled to 0° C. and 150 mg (0.39 mmol) of Ph-I=N-Ses reagent are added, and the mixture is again stirred at room temperature. This procedure is repeated 3 times over the course of a further 18 h, and a total of 616 mg (1.61 mmol) of Ph-I=N-Ses reagent and 130 mg (0.35 mmol) of CuPF₆(MeCN)₄ are added. The mixture is diluted with ethyl acetate and washed with saturated NaCl solution. The aqueous phase is again extracted with ethyl acetate (2×). The combined organic phases are dried (Na₂SO₄), filtered and concentrated. The remaining residue is purified by chromatography (hexane/EtOAc 4:1). 280 mg (0.44 mmol, corresponding to 67% of theory) of the product are obtained.

¹H-NMR (DMSO): 8.22 (s, 1H), 8.10 (m, 1H), 8.05 (m, 2H), 7.72 (t, 1H), 4.39 (m, 2H), 3.65 (s, 3H), 3.42 (m, 2H), 2.90 (m, 2H), 1.82 (m, 4H), 0.91 (m, 2H), −0.02 (s, 9H).

MS: 642 (ES).
e) Compound 9.5

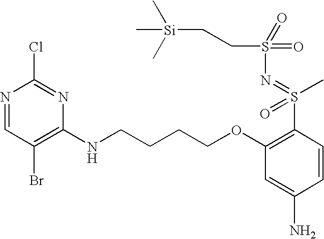

3.0 ml of an approx. 10% strength solution of titanium(III) chloride in 20-30% strength hydrochloric acid are added to a solution of 280 mg (0.44 mmol) of compound 9.4 in 20 ml of THF under argon at room temperature. After 20 min, another 0.5 ml of the titanium(III) chloride solution is added to the reaction solution, which is stirred for a further 4 hours. Another 0.5 ml of the reducing agent is added, and the mixture is stirred overnight. After a TLC check, 0.3 ml of the titanium(III) chloride solution is added. After 2 hours, the mixture is diluted with ethyl acetate and basified with 1N NaOH solution. The phases are separated and the aqueous phase is again extracted with ethyl acetate. The combined organic phases are dried (Na₂SO₄), filtered and concentrated. The remaining residue is purified by chromatography (DCM/EtOH 9:1). 184 mg (0.30 mmol, corresponding to 69% of theory) of the product are obtained.

¹H-NMR (DMSO): 8.22 (s, 1H), 7.73 (t, 1H), 7.42 (m, 1H), 6.35 (m, 4H), 4.05 (m, 2H), 3.40 (m, 5H), 2.80 (m, 2H), 1.78 (m, 4H), 0.88 (m, 2H), −0.02 (s, 9H).

MS: 612 (ES).

2. Preparation of the Final Product

A solution of 178 mg (0.29 mmol) of compound 9.5 in acetonitrile/water/n-butanol (9 ml/1 ml/3 ml) is added by means of a syringe driver over the course of 4 hours to a refluxing solution of acetonitrile/water/4 N solution of hydrogen chloride in dioxane (45 ml/5 ml/0.5 ml). After 10 days, the mixture is diluted with water and extracted with ethyl acetate (2×). The aqueous phase is neutralized with NaHCO₃ solution and again extracted with ethyl acetate (2×). The combined organic phases are dried (Na₂SO₄), filtered and concentrated. The resulting residue is purified by chromatography (DCM/EtOH 9:1). 80 mg (0.14 mmol, corresponding to 48% of theory) of the product are obtained.

¹H-NMR (DMSO): 9.98 (s, 1H), 9.24 (m, 1H), 8.10 (s, 1H), 7.61 (m, 1H), 7.55 (t, 1H), 6.85 (m, 1H), 4.53 (m, 2H), 3.53 (s, 3H), 3.40 (m, 2H), 2.80 (m, 2H), 1.91 (m, 4H), 0.91 (m, 2H), −0.02 (s, 9H).

MS: 576 (ES).

### EXAMPLE 10

### (RS)—S-[1⁵-Bromo-4-oxa-2,9-diaza-1(2,4)-pyrimidina-3(1,3)benzenacyclononaphan-3⁴-yl]-N-(methylcarbamoyl)-S-methylsulphoximide

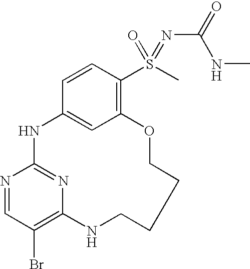 1. Preparation of the Intermediates
a) Compound 10.1

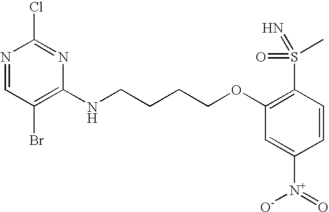

280 mg (4.3 mmol) of sodium azide are added in portions to a stirred mixture of 2.0 g (4.3 mmol) of compound 9.3 in 10 ml of fuming sulphuric acid (oleum, Riedel de Haen, 20% SO₃) at 0° C. The mixture is stirred at 45° C. for one hour and again cooled to 0° C., and a further 130 mg (2.0 mmol) of sodium azide are added. The mixture is stirred at 45° C. for 30 minutes and, after cooling, added to ice-water. The mixture is basified with NaHCO₃ solution and extracted with ethyl acetate (2×). The combined organic phases are dried (Na₂SO₄), filtered and concentrated. 1.95 g (4.1 mmol; corresponding to 94% of theory) of the product are obtained.

¹H-NMR (DMSO): 8.19 (s, 1H), 8.05 (m, 1H), 7.91 (m, 2H), 7.77 (tr, 1H), 4.57 (br, 1H), 4.30 (m, 2H), 3.40 (m, 2H), 3.17 (s, 3H), 1.77 (m, 4H).

MS (ES): 478.
b) Compound 10.2

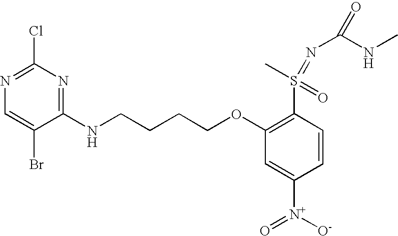

0.025 ml (0.42 mmol) of methyl isocyanate is added to a solution of 200 mg (0.42 mmol) of compound 10.1 in 5 ml of DMF and 0.058 ml (0.42 mmol) of triethylamine at room temperature, and the mixture is stirred at room temperature for 24 hours. 0.025 ml (0.42 mmol) of methyl isocyanate is again added to the mixture, which is stirred for a further 24 hours. The mixture is mixed with NaCl solution and extracted with ethyl acetate (2×). The combined organic phases are washed with 1N HCl, saturated NaHCO₃ solution and NaCl solution, dried (Na₂SO₄), filtered and concentrated. 206 mg (0.38 mmol; corresponding to 92% of theory) of the product are obtained.

¹H-NMR (DMSO): 8.24 (s, 1H), 8.10 (m, 1H), 7.98 (m, 2H), 7.77 (tr, 1H), 6.77 (q, 1H), 4.34 (m, 2H), 3.40 (m, 5H), 2.43 (d, 3H), 1.78 (m, 4H).

MS (ES): 535 (ES).
c) Compound 10.3

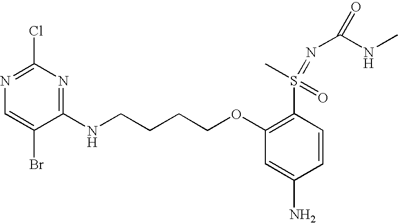

1.9 ml of a 15% strength solution of titanium(III) chloride in approx. 10% strength hydrochloric acid are added to a solution of 200 mg (0.37 mmol) of compound 10.2 in 5.5 ml of THF at 0° C. The mixture is stirred at room temperature for 4 hours. The mixture is basified with 2N NaOH solution while cooling in ice and, after addition of solid NaCl, extracted with ethyl acetate (2×). The combined organic phases are washed with NaCl solution, dried (Na₂SO₄), filtered and concentrated. 169 mg (0.33 mmol; corresponding to 90% of theory) of the product are obtained.

¹H-NMR (DMSO): 8.19 (s, 1H), 7.74 (tr, 1H), 7.40 (m, 1H), 6.39 (br, 1H), 6.18 (m, 2H), 6.00 (s, 2H), 3.96 (m, 2H), 3.38 (m, 2H), 3.29 (s, 3H), 2.41 (d, 3H), 1.70 (m, 4H).

MS: 505 (ES)

2. Preparation of the Final Product

a) Cyclization Under Acidic Conditions:

20 mg (0.040 mmol) of compound 10.3 are mixed with 4 ml of water and 0.1 ml of a 4N solution of hydrogen chloride in dioxane. The reaction vessel is closed and the mixture is heated in a microwave (Biotage Initiator) at 120° C. for 30 minutes.

The mixture is analysed by HPLC MS:

Column: Acquity UPLC BEH C18; 1.7 μm; 2.1×50 mm

Eluents: A: H₂O+0.1% TFA; B: MeCN; 1%->99% B in 1.7 min

Flow rate: 0.8 ml/min

Detector: UV DAD (200-400 nM) TAC
MS ESI+ (160-800 Da) TIC

Temperature: 60° C.

Retention: 0.68 min (mass found: 468.1)

The mixture is filtered and concentrated. 8 mg (0.017 mmol, corresponding to 43% of theory) of the product are obtained.

¹H-NMR (DMSO): 10.64 (s, 1H), 8.86 (m, 1H), 8.38 (m, 1H), 8.27 (s, 1H), 7.73 (m, 2H), 6.75 (m, 1H), 4.39 (m, 2H), 3.41 (m, 2H), 3.35 (s, 3H), 2.45 (d, 3H), 1.65 (m, 4H).

MS: 469 (ES).

b) Cyclization Under Neutral Conditions:

20 mg (0.040 mmol) of compound 10.3 are mixed with 4 ml of water. The reaction vessel is closed and the mixture is heated in a microwave (Biotage Initiator) at 120° C. for 60 minutes.

The mixture is analysed by HPLC MS:

Column: Acquity UPLC BEH C18; 1.7 μm, 2.1×50 mm

Eluents: A: H₂O+0.1% TFA; B: MeCN; 1%->99% B in 1.7 min

Flow rate: 0.8 ml/min

Detector: UV DAD (200-400 nM) TAC
MS ESI+ (160-800 Da) TIC

Temperature: 60° C.

Retention: 0.68 min (mass found: 468.1)

The mixture is purified by HPLC.

### EXAMPLE 11

### (RS)—S-[1⁵-Bromo-4-oxa-2,9-diaza-1(2,4)-pyrimidina-3(1,3)-benzenacyclononaphan-3⁴-yl]-S-methyl-N-(phenyl-carbamoyl)sulphoximide

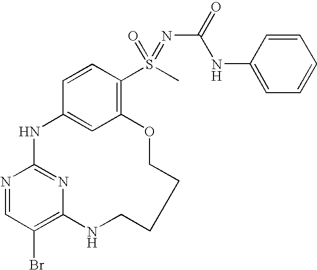 1. Preparation of the Intermediates
a) Compound 11.1

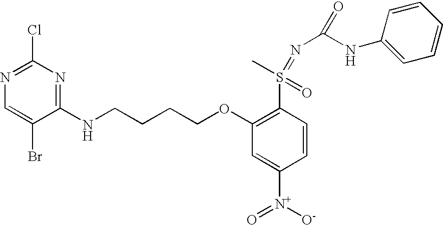

0.045 ml (0.42 mmol) of phenyl isocyanate is added to a solution of 200 mg (0.42 mmol) of compound 10.1 in 5 ml of DMF and 0.058 ml (0.42 mmol) of triethylamine at room temperature, and the mixture is stirred at room temperature for 4 hours. The mixture is mixed with NaCl solution and extracted with ethyl acetate. The combined organic phases are dried (Na₂SO₄), filtered and concentrated. 273 mg of the crude product are obtained.

MS (ES): 597.
b) Compound 11.2

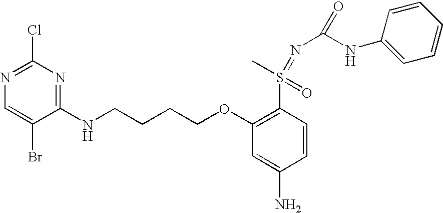

2.3 ml of a 15% strength solution of titanium (III) chloride in approx. 10% strength hydrochloric acid are added to a solution of 265 mg (0.44 mmol) of compound 11.1 in 6.5 ml of THF at 0° C. The mixture is stirred at room temperature for 4 hours. The mixture is basified with 2N NaOH solution while cooling in ice and, after addition of solid NaCl, extracted with ethyl acetate (2×). The combined organic phases are washed with NaCl solution, dried (Na₂SO₄), filtered and concentrated. The resulting residue is purified by chromatography (DCM/EtOH 9:1). 153 mg (0.27 mmol; corresponding to 61% of theory) of the product are obtained.

¹H-NMR (DMSO): 9.06 (s, 1H), 8.22 (s, 1H), 7.72 (tr, 1H), 7.48 (m, 3H), 7.16 (m, 2H), 6.86 (m, 1H), 6.26 (m, 2H), 6.11 (s, 2H), 4.00 (m, 2H), 3.40 (m, 5H), 1.75 (m, 4H).

MS: 567 (ES).

2. Preparation of the Final Product

10 mg (0.04 mmol) of compound 11.2 are mixed with 2 ml of water and 0.05 ml of a 4N solution of hydrogen chloride in dioxane. The reaction vessel is closed and the mixture is heated in a microwave (Biotage Initiator) at 120° C. for 30 minutes.

The mixture is analysed by HPLC MS:

Column: Acquity UPLC BEH C18; 1.7 μm; 2.1×50 mm

Eluents: A: H₂O+0.1% TFA; B: MeCN; 1%->99% B in 1.7 min

Flow rate: 0.8 ml/min

Detector: UV DAD (200-400 nM) TAC
MS ESI+ (160-800 Da) TIC

Temperature: 60° C.

Retention: 0.92 min (mass found: 530.07)

The mixture is purified by HPLC.

### EXAMPLE 12

### (RS)—S-[1⁵-Bromo-4-oxa-2,9-diaza-1(2,4)-pyrimidina-3(1,3)-benzenacyclononaphan-3⁴-yl]-S-methyl-N-(3-pyridyl-carbamoyl)sulphoximide

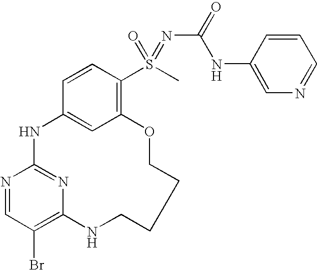 1. Preparation of the Intermediates
a) Compound 12.1

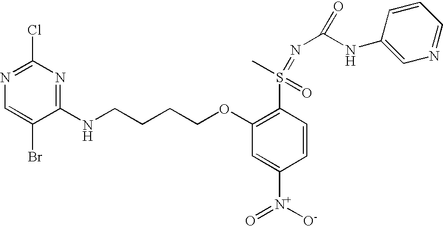

50 mg (0.42 mmol) of 3-isocyanate-pyridine are added to a solution of 200 mg (0.42 mmol) of compound 10.1 in 5 ml of DMF and 0.058 ml (0.42 mmol) of triethylamine at room temperature, and the mixture is stirred at room temperature for 24 hours. 25 mg (0.21 mmol) of 3-isocyanate-pyridine are again added to the mixture, which is stirred for a further 24 hours. The mixture is mixed with saturated NaHCO₃ solution and extracted with ethyl acetate (2×). The combined organic phases are dried (Na₂SO₄), filtered and concentrated. The resulting residue is purified by chromatography (DCM/EtOH 9:1). 167 mg (0.28 mmol; corresponding to 67% of theory) of the product are obtained.

¹H-NMR (DMSO): 9.57 (br, 1H), 8.59 (m, 1H), 8.21 (s, 1H), 8.18 (m, 1H), 8.06 (m, 3H), 7.83 (m, 1H), 7.70 (tr, 1H), 7.18 (m, 1H), 4.34 (m, 2H), 3.55 (s, 3H), 3.37 (m, 2H), 1.78 (m, 4H).

MS: 598 (ES)
b) Compound 12.2

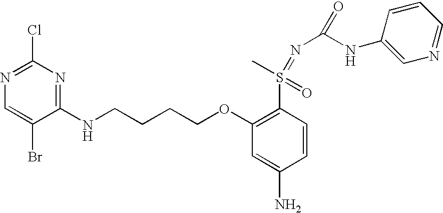

1.4 ml of a 15% strength solution of titanium(III) chloride in approx. 10% strength hydrochloric acid are added to a solution of 160 mg (0.27 mmol) of compound 12.1 in 4.0 ml of THF at 0° C. The mixture is stirred at room temperature for 4 hours. The mixture is basified with 2N NaOH solution while cooling with ice and, after addition of solid NaCl, extracted with ethyl acetate (2×). The combined organic phases are washed with NaCl solution, dried (Na₂SO₄), filtered and concentrated. 144 mg (0.25 mmol; corresponding to 95% of theory) of the product are obtained.

¹H-NMR (DMSO): 9.25 (s, 1H), 8.58 (m, 1H), 8.16 (s, 1H), 8.02 (m, 1H), 7.85 (m, 1H), 7.66 (m, 1H), 7.47 (m, 1H), 7.14 (m, 1H), 7.24 (m, 2H), 6.09 (s, 2H), 4.00 (m, 2H), 3.38 (m, 5H), 1.69 (m, 4H).

MS: 568 (ES)

2. Preparation of the Final Product

10 mg (0.04 mmol) of compound 12.2 are mixed with 2 ml of water and 0.05 ml of 4N solution of hydrogen chloride in dioxane. The reaction vessel is closed and the mixture is heated in a microwave (Biotage Initiator) at 120° C. for 30 minutes.

The mixture is analysed by HPLC MS:

Column: Acquity UPLC BEH C18; 1.7 μm; 2.1×50 mm

Eluents: A: H₂O+0.1% TFA; B: MeCN; 1%->99% B in 1.7 min

Flow rate: 0.8 ml/min

Detector: UV DAD (200-400 nM) TAC
MS ESI+ (160-800 Da) TIC

Temperature: 60° C.

Retention: 0.68 min (mass found: 532.4)

The mixture is purified by HPLC.

### EXAMPLE 13

### (RS)—S-[1⁵-Bromo-4-oxa-2,9-diaza-1(2,4)-pyrimidina-3(1,3)-benzenacyclononaphan-3⁴-yl]-N-{[4-(dimethylamino)phenyl]-carbamoyl}-S-methylsulphoximide

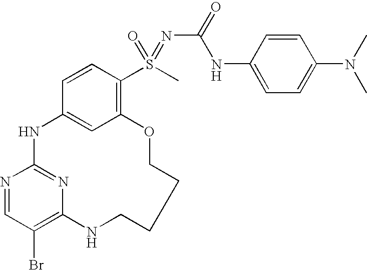 1. Preparation of the Intermediates
a) Compound 13.1

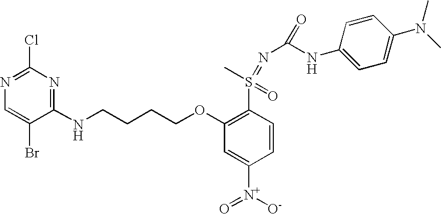

68 mg (0.42 mmol) of (4-isocyanatophenyl)dimethylamine are added to a solution of 200 mg (0.42 mmol) of compound 10.1 in 5 ml of DMF and 0.058 ml (0.42 mmol) of triethylamine at room temperature, and the mixture is stirred at room temperature for 24 hours. The mixture is mixed with NaCl solution and extracted with ethyl acetate (2×). The combined organic phases are washed with 1N HCl, saturated NaHCO₃ solution and NaCl solution, dried (Na₂SO₄), filtered and concentrated. The resulting residue is purified by chromatography (DCM/EtOH 9:1). 128 mg (0.20 mmol; corresponding to 48% of theory) of the product are obtained.

¹H-NMR (DMSO): 8.99 (br, 1H), 8.23 (s, 1H), 8.16 (m, 1H), 8.04 (m, 1H), 7.99 (m, 1H), 7.70 (tr, 1H), 7.22 (m, 2H), 6.57 (m, 2H), 4.35 (m, 2H), 3.49 (s, 3H), 3.40 (m, 2H), 2.78 (s, 6H), 1.79 (m, 4H).

MS (ES): 640 (ES).
b) Compound 13.2

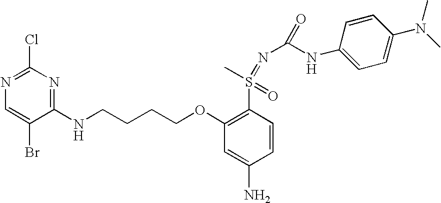

1.4 ml of a 15% strength solution of titanium(111) chloride in approx. 10% strength hydrochloric acid are added to a solution of 120 mg (0.19 mmol) of compound 13.1 in 2.8 ml of THF at 0° C. The mixture is stirred at room temperature for 4 hours. The mixture is basified with 2N NaOH solution while cooling in ice and, after addition of solid NaCl, extracted with ethyl acetate (2×). The combined organic phases are washed with NaCl solution, dried (Na₂SO₄), filtered and concentrated. 110 mg (0.18 mmol; corresponding to 96% of theory) of the product are obtained.

¹H-NMR (DMSO): 8.65 (s, 1H), 8.17 (s, 1H), 7.72 (tr, 1H), 7.47 (m, 1H), 7.23 (m, 2H), 6.54 (m, 2H), 6.21 (m, 2H), 6.04 (s, 2H), 3.98 (m, 2H), 3.35 (m, 5H), 2.74 (s, 6H), 1.70 (m, 4H).

MS: 610 (ESI).

2. Preparation of the Final Product

10 mg (0.04 mmol) of compound 13.2 are mixed with 2 ml of water and 0.05 ml of a 4N solution of hydrogen chloride in dioxane. The reaction vessel is closed and the mixture is heated in a microwave (Biotage Initiator) at 120° C. for 30 minutes.

The mixture is analysed by HPLC MS:

Column: Acquity UPLC BEH C18; 1.7 μm; 2.1×50 mm

Eluents: A: H₂O+0.1% TFA; B: MeCN; 1%->99% B in 1.7 min

Flow rate: 0.8 ml/min

Detector: UV DAD (200-400 nM) TAC
MS ESI+ (160-800 Da) TIC

Temperature: 60° C.

Retention: 0.70 min (mass found: 574.5)

The mixture is purified by HPLC.

### EXAMPLE 14

### (RS)—N-(Allylcarbamoyl)-S-[1⁵-bromo-4-oxa-2,9-diaza-1(2,4)-pyrimidina-3(1,3)-benzenacyclononaphan-3⁴-yl]-S-methyl-sulphoximide

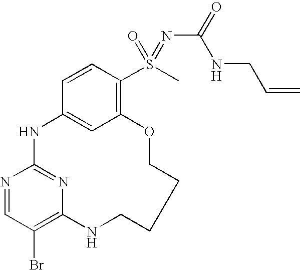 1. Preparation of the Intermediates
a) Compound 14.1

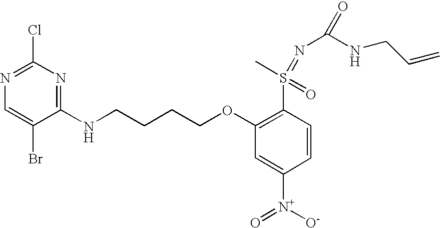

35 mg (0.42 mmol) of allyl isocyanate are added to a solution of 200 mg (0.42 mmol) of compound 10.1 in 5 ml of DMF and 0.058 ml (0.42 mmol) of triethylamine at room temperature, and the mixture is stirred at room temperature for 24 hours. 17 mg (0.21 mmol) of allyl isocyanate are again added to the mixture, which is stirred for a further 24 hours. The mixture is mixed with NaCl solution and extracted with ethyl acetate. The combined organic phases are washed with 1N HCl, saturated NaHCO₃ solution and NaCl solution, dried (Na₂SO₄), filtered and concentrated. The resulting residue is purified by chromatography (DCM/EtOH 9:1). 160 mg (0.28 mmol; corresponding to 68% of theory) of the product are obtained.

¹H-NMR (DMSO): 8.24 (s, 1H), 8.11 (m, 1H), 7.99 (m, 2H), 7.77 (tr, 1H), 7.04 (tr, 1H), 5.48 (m, 1H), 4.99 (m, 2H), 4.34 (m, 2H), 3.40 (m, 7H), 1.80 (m, 4H).

MS (ES): 561 (ES).
b) Compound 14.2

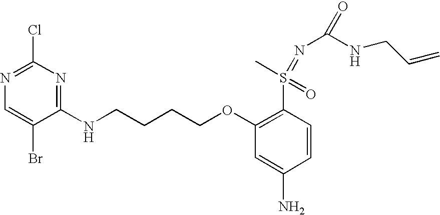

1.4 ml of a 15% strength solution of titanium(111) chloride in approx. 10% strength hydrochloric acid are added to a solution of 152 mg (0.27 mmol) of compound 14.1 in 4.0 ml of THF at 0° C. The mixture is stirred at room temperature for 4 hours. The mixture is basified with 2N NaOH solution while cooling in ice and, after addition of solid NaCl, extracted with ethyl acetate (2×). The combined organic phases are washed with NaCl solution, dried (Na₂SO₄), filtered and concentrated. 144 mg (0.27 mmol; corresponding to 100% of theory) of the product are obtained.

¹H-NMR (DMSO): 8.19 (s, 1H), 7.73 (tr, 1H), 7.41 (m, 1H), 6.68 (tr, 1H), 6.21 (m, 1H), 6.17 (m, 1H), 6.00 (s, 2H), 5.68 (m, 1H), 5.03 (m, 1H), 4.91 (m, 1H), 3.95 (m, 2H), 3.49 (m, 2H), 3.37 (m, 2H), 3.29 (s, 3H), 1.70 (m, 4H).

MS: 531 (ESI).

2. Preparation of the Final Product

10 mg (0.04 mmol) of compound 14.2 are mixed with 2 ml of water and 0.05 ml of a 4N solution of hydrogen chloride in dioxane. The reaction vessel is closed and the mixture is heated in a microwave (Biotage Initiator) at 120° C. for 30 minutes.

The mixture is analysed by HPLC MS:

Column: Acquity UPLC BEH C18; 1.7 μm; 2.1×50 mm

Eluents: A: H₂O+0.1% TFA; B: MeCN; 1%->99% B in 1.7 min

Flow rate: 0.8 ml/min

Detector: UV DAD (200-400 nM) TAC
MS ESI+ (160-800 Da) TIC

Temperature: 60° C.

Retention: 0.78 min (mass found: 495.4)

The mixture is purified by HPLC.

### EXAMPLE 15

### (RS)—S-[1⁵-Bromo-4-oxa-2,9-diaza-1(2,4)-pyrimidina-3(1,3)-benzenacyclononaphan-3⁴-yl]-N-(cyclopentylcarbamoyl)-S-methylsulphoximide

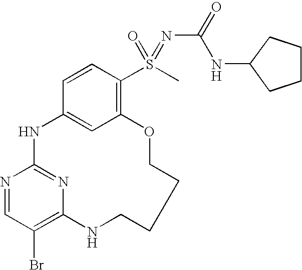 1. Preparation of the Intermediates
a) Compound 15.1

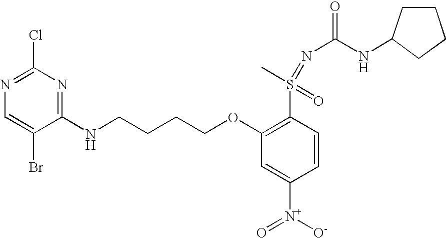

0.047 ml (0.42 mmol) of cyclopentyl isocyanate is added to a solution of 200 mg (0.42 mmol) of compound 10.1 in 5 ml of DMF and 0.058 ml (0.42 mmol) of triethylamine at room temperature, and the mixture is stirred at room temperature for 24 hours. 0.024 mg (0.21 mmol) of cyclopentyl isocyanate is again added to the mixture, which is stirred for a further 24 hours. The mixture is mixed with NaCl solution and extracted with ethyl acetate (2×). The combined organic phases are washed with 1N HCl, saturated NaHCO₃ solution and NaCl solution, dried (Na₂SO₄), filtered and concentrated. The resulting residue is purified by chromatography (DCM/EtOH 9:1). 117 mg (0.20 mmol; corresponding to 48% of theory) of the product are obtained.

¹H-NMR (DMSO): 8.25 (s, 1H), 8.10 (m, 1H), 7.96 (m, 2H), 7.60 (m, 1H), 5.67 (d, 1H), 4.31 (m, 2H), 3.83 (p, 1H), 3.42 (m, 5H), 1.50 (m, 12H).
b) Compound 15.2

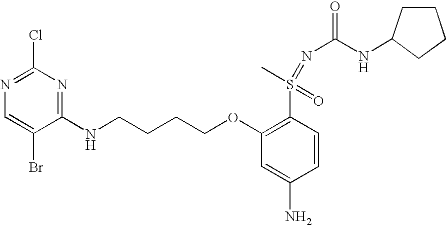

Compound 15.1 can be reduced with Ti(III) chloride to the desired product 15.2 in analogy to the method described for compound 14.2

2. Preparation of the Final Product

Compound 15.2 can be cyclized to the desired product 15 in a microwave in analogy to the methods described in Example 10.

### EXAMPLE 16

Further intermediates which can be used to prepare products according to the invention by a process variant 2:
Compound 16.1

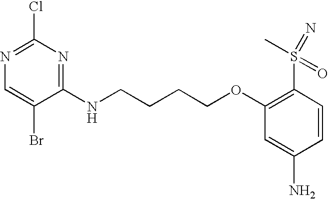

2.0 ml of a 15% strength solution of titanium(III) chloride in approx. 10% strength hydrochloric acid are added to a solution of 190 mg (0.40 mmol) of compound 10.1 in 20 ml of THF at room temperature. The mixture is stirred at room temperature for 2 hours. The mixture is diluted with ethyl acetate, made slightly basic with NaHCO₃ solution and then extracted with ethyl acetate (2×). The combined organic phases are washed with NaCl solution, dried (Na₂SO₄), filtered and concentrated. 154 mg (0.34 mmol; corresponding to 86% of theory) of the product are obtained.

¹H-NMR (DMSO): 8.19 (s, 1H), 7.77 (tr, 1H), 7.40 (m, 1H), 6.20 (m, 1H), 6.12 (m, 1H), 8.82 (br, 2H), 3.98 (m, 2H), 3.60 (s, 1H), 3.40 (m, 2H), 2.99 (s, 3H), 1.72 (m, 4H).

MS: 449 (E1).
b) Compound 16.2

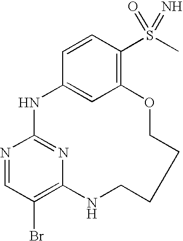

95 mg (0.21 mmol) of compound 16.1 are mixed with 19 ml of water and 0.34 ml of a 4N solution of hydrogen chloride in dioxane. The reaction vessel is closed and the mixture is heated in a microwave (Biotage Initiator) at 130° C. for 2 hours. After cooling, the mixture is diluted with ethyl acetate and basified with 2N NaOH solution. The mixture is extracted with ethyl acetate (3×). The combined organic phases are dried (Na₂SO₄), filtered and concentrated. The resulting residue is purified by chromatography (DCM/EtOH 9:1). 9 mg (0.02 mmol; corresponding to 10% of theory) of the product are obtained.

¹H-NMR (DMSO): 9.76 (s, 1H), 9.13 (br, 1H), 8.08 (s, 1H), 7.62 (m, 1H), 7.50 (tr, 1H), 6.75 (m, 1H), 4.46 (m, 2H), 3.94 (s, 1H), 3.44 (m, 2H), 3.10 (s, 3H), 1.88 (m, 4H).

MS: 412 (ES).

Compound 16.2 can be converted by N-functionalization of the sulphoximine by process variant 1, c₁) to compounds according to the invention:
Alkylation (see, for example: C. R. Johnson, J. Org. Chem. 1993, 58, 1922-1923).
Acylation (see, for example: a) C. P. R. Hackenberger, G. Raabe, C. Bolm, Chem. Europ. J. 2004, 10, 2942-2952; b) C. Bolm, C. P. R. Hackenberger, O. Simic, M. Verrucci, D. Müller, F. Bienewald, Synthesis 2002, 7, 879-887; c) C. Bolm, G. Moll, J. D. Kahmann, Chem. Europ. J. 2001, 7, 1118-1128).
Arylation (see, for example: a) C. Bolm, J. P. Hildebrand, Tetrahedron Lett. 1998, 39, 5731-5734; b) C. Bolm, J. P. Hildebrand, J. Org. Chem. 2000, 65, 169-175; c) C. Bolm, J. P. Hildebrand, J. Rudolph, Synthesis 2000, 7, 911-913; d) Y. C. Gae, H. Okamura, C. Bolm, J. Org. Chem. 2005, 70, 2346-2349).
Reaction with isocyanates/isothiocyanates (see, for example: a) V. J. Bauer, W. J. Fanshawe, S. R. Safir, J. Org. Chem. 1966, 31, 3440-3441; b) C. R. Johnson, M. Haake, C. W. Schroeck, J. Am. Chem. Soc. 1970, 92, 6594-6598; c) S. Allenmark, L. Nielsen, W. H. Pirkle, Acta Chem. Scand. Ser. B 1983, 325-328).
Reaction with sulphonyl chlorides (see, for example: a) D. J. Cram, J. Day, D. R. Rayner, D. M. von Schriltz, D. J. Duchamp, D. C. Garwood, J. Am. Chem. Soc. 1970, 92, 7369-7384), b) C. R. Johnson, H. G. Corkins, J. Org. Chem. 1978, 43, 4136-4140; c) D. Craig, N. J. Geach, C. J. Pearson, A. M. Z. Slawin, A. J. P. White, D. J. Williams, Tetrahedron 1995, 51, 6071-6098).
Reaction with chloroformates or anhydrides (see, for example: a) D. J. Cram, J. Day, D. R. Rayner, D. M. von Schriltz, D. J. Duchamp, D. C. Garwood, J. Am. Chem. Soc. 1970, 92, 7369-7384), b) S. G. Pyne, Z. Dong, B. W. Skelton, A. H. Allan, J. Chem. Soc. Chem. Commun. 1994, 6, 751-752; c) C. R. Johnson, H. G. Corkins, J. Org. Chem. 1978, 43, 4136-4140; d) Y. C. Gae, H. Okamura, C. Bolm, J. Org. Chem. 2005, 2346-2349).
Silylation: (see, for example: A. J. Pearson, S. L. Blystone, H. Nar, A. A. Pinkerton, B. A. Roden, J. Yoon, J. Am. Chem. Soc. 1989, 111, 134-144).
   Process Variant 3

### EXAMPLE 17

### (RS)—N-(Ethoxycarbonyl)-S-methyl-S-[1⁵-3-pyridyl-2,4,9-triaza-1(2,4)-pyrimidina-3(1,3)-benzenacyclononaphan-3⁴-yl]sulphoximide

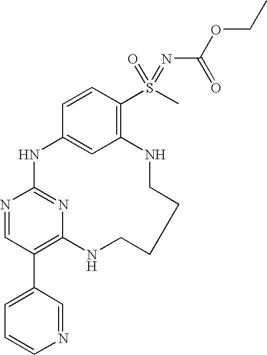

1.6 ml of a 0.5 molar sodium hydroxide solution are added to 150 mg (0.28 mmol) of (RS)—N-(ethoxycarbonyl)-S-[1⁵-iodo-2,4,9-triaza-1(2,4)-pyrimidina-3(1,3)-benzenacyclononaphan-3⁴-yl]-S-methylsulphoximide (Example 4), 52 mg (0.42 mmol) of 3-pyridineboronic acid and 122 mg (0.11 mmol) of palladium tetrakistriphenylphosphine in 5 ml of dimethoxyethane under argon. The mixture is flushed with argon and heated to 90° C. After 90 minutes, the mixture is cooled and added to a saturated NaCl solution. The mixture is extracted with ethyl acetate. The combined organic phases are dried (Na₂SO₄), filtered and concentrated. The resulting residue is purified by chromatography.

MS: 482 (ES+)

Assay 1

Aurora-C Kinase Assay

Recombinant Aurora-C protein was expressed in transiently transfected HEK293 cells and then purified. The kinase substrate used was the biotinylated peptide having the amino acid sequence biotin-FMRLRRLSTKYRT, which was purchased from Jerini A G in Berlin.

Aurora-C [5 nM in the test mixture, test volume 5 μl] was incubated in the presence of various concentrations of test substances (0 μM and 10 measurement points within the range 0.001-20 μM in duplicate) in assay buffer [25 mM HEPES pH 7.4, 0.5 mM MnCl₂, 0.1 mM Na ortho-vanadate, 2.0 mM dithiothreitol, 0.05% bovine serum albumin (BSA), 0.01% Triton X-100, 3 μM adenosine trisphosphate (ATP), 0.67 nCi/μl gama-P33-ATP, 2.0 μM substrate peptide biotin-FMRLRRLSTKYRT, 1.0% dimethyl sulphoxide] at 22° C. for 90 min. The reaction was stopped by adding 12.5 μl of an EDTA/detection solution [16 mM EDTA, 40 mM ATP, 0.08% Triton X-100, 4 mg/ml PVT streptavidin SPA beads (from Amersham)]. After incubation for 10 minutes, the SPA beads were pelleted by centrifugation at 1000×G for 10 minutes. Measurement took place in a PerkinElmer Topcount scintillation counter. The measured data were normalized to 0% inhibition (enzyme reaction without inhibitor) and 100% inhibition (enzyme reaction in the presence of 0.1 μM staurosporine (from Sigma)). The IC50 values were determined by means of a 4-parameter fit using the company's own software.

Assay 2

Aurora-A Kinase Assay

Recombinant Aurora-A protein, expressed in Sf21 insect cells, was purchased from Upstate. The kinase substrate used was the biotinylated peptide having the amino acid sequence biotin-LNYNRRLSLGPMF, which was purchased from Jerini A G in Berlin.

Aurora-A [15 nM in the test mixture, test volume 5 μl] was incubated in the presence of various concentrations of test substances (0 μM and 10 measurement points within the range 0.001-20 μM in duplicate) in assay buffer [25 mM HEPES pH 7.4, 3 mM MnCl₂, 5 mM MnCl₂, 0.1 mM Na ortho-vanadate, 2.0 mM dithiothreitol, 0.05% bovine serum albumin (BSA), 0.01% Triton X-100, 8 μM ATP, 4 nCi/μl gama-P33-ATP, 5.0 μM substrate peptide biotin-LNYNRRLSLGPMF, 1.0% dimethyl sulphoxide] at 22° C. for 90 min. The reaction was stopped by adding 12.5 μl of an EDTA/detection solution [16 mM EDTA, 40 mM ATP, 0.08% Triton X-100, 4 mg/ml PVT streptavidin SPA beads (from Amersham)]. After incubation for 10 minutes, the SPA beads were pelleted by centrifugation at 1000×G for 10 minutes. Measurement took place in a PerkinElmer Topcount scintillation counter.

The measured data were normalized to 0% inhibition (enzyme reaction without inhibitor) and 100% inhibition (enzyme reaction in the presence of 0.1 μM staurosporine (from Sigma)). The IC50 values were determined by means of a 4-parameter fit using the company's own software.

Assay 3

CDK1/CycB Kinase Assay

Recombinant CDK1- and CycB-GST fusion proteins, purified from baculovirus-infected insect cells (Sf9), were purchased from ProQinase GmbH, Freiburg. The histone IIIS used as kinase substrate can be purchased from Sigma. CDK1/CycB (200 ng/measurement point) was incubated in the presence of various concentrations of test substances (0 μM, and within the range 0.01-100 μM) in assay buffer [50 mM Tris/HCl pH 8.0, 10 mM MgCl2, 0.1 mM Na ortho-vanadate, 1.0 mM dithiothreitol, 0.5 μM ATP, 10 μg/measurement point histone IIIS, 0.2 μCi/measurement point 33P-gamma ATP, 0.05% NP40, 1.25% dimethyl sulphoxide] at 22° C. for 10 min. The reaction was stopped by adding EDTA solution (250 mM, pH 8.0, 15 μl/measurement point).

15 μl of each reaction mixture were loaded onto P30 filter strips (from Wallac), and non-incorporated 33P-ATP was removed by washing the filter strips three times in 0.5% strength phosphoric acid for 10 min each time. After the filter strips had been dried at 70° C. for 1 hour, the filter strips were covered with scintillator strips (MeltiLex™ A, from Wallac) and baked at 90° C. for 1 hour. The amount of incorporated 33P (substrate phosphorylation) was determined by scintillation measurement in a gamma radiation counter (Wallac).

Assay 4

CDK2/CycE Kinase Assay

Recombinant CDK2- and CycE-GST fusion proteins, purified from baculovirus-infected insect cells (Sf9), were purchased from ProQinase GmbH, Freiburg. The histone IIIS used as kinase substrate was purchased from Sigma. CDK2/CycE (50 ng/measurement point) was incubated in the presence of various concentrations of test substances (0 μM, and within the range 0.01-100 μM) in assay buffer [50 mM Tris/HCl pH 8.0, 10 mM MgCl₂, 0.1 mM Na ortho-vanadate, 1.0 mM dithiothreitol, 0.5 μM ATP, 10 μg/measurement point histone IIIS, 0.2 μCi/measurement point ³³P-gamma ATP, 0.05% NP40, 1.25% dimethyl sulphoxide] at 22° C. for 10 min. The reaction was stopped by adding EDTA solution (250 mM, pH 8.0, 15 μl/measurement point).

15 μl of each reaction mixture were loaded onto P30 filter strips (from Wallac), and non-incorporated ³³P-ATP was removed by washing the filter strips three times in 0.5% strength phosphoric acid for 10 min each time.

After the filter strips had been dried at 70° C. for 1 hour, the filter strips were covered with scintillator strips (MeltiLex™ A, from Wallac) and baked at 90° C. for 1 hour. The amount of incorporated ³³P (substrate phosphorylation) was determined by scintillation measurement in a gamma radiation counter (Wallac).

Assay 5

Chk1 Kinase Assay

Recombinant Chk1 protein was expressed in Sf9 insect cells and then purified. The kinase substrate used was the biotinylated peptide having the amino acid sequence biotin-ALKLVRTPSFVITAK, which was purchased from Biosynthan GmbH in Berlin.

Chk1 [0.11 μg/ml in the test mixture, test volume 5 μl] was incubated in the presence of various concentrations of test substances (0 μM, and 10 measurement points within the range 0.001-20 μM in duplicate) in assay buffer [50 mM HEPES pH 7.5, 10 mM MgCl₂, 1.0 mM MgCl₂, 0.1 mM Na ortho-vanadate, 1.0 mM dithiothreitol, 1 tablet/2.5 ml complete protease inhibitor (from Roche), 10 μM ATP, 1.0 μM substrate peptide biotin-ALKLVRTPSFVITAK, 1.0% dimethyl sulphoxide] at 22° C. for 60 min. The reaction was stopped by adding 5 μl of an EDTA/detection solution [100 mM EDTA, 800 mM potassium fluoride, 0.2% BSA, 0.2 μM streptavidin-XLent (from CisBio), 9.6 nM anti-phospho-Akt antibody (from Cell Signalling Technology), 4 nM protein-A-Eu(K) (from CisBio)]. The fluorescence emission at 620 nm and 665 nm after excitation with light of the wavelength 350 nm was measured in a Rubystar HTRF instrument from BMG Labsystems.

The measured data (ratio of emission 665 divided by emission 620 multiplied by 10 000) were normalized to 0% inhibition (enzyme reaction without inhibitor) and 100% inhibition (all assay components apart from enzyme). The IC50 values were determined by means of a 4-parameter fit using the company's own software.

Assay 6

c-Kit Kinase Assay

Recombinant c-kit protein was expressed in E. coli and then purified. The kinase substrate used was the biotinylated peptide having the amino acid sequence biotin-poly GluTyr, which was purchased from CisBio.

C-kit [test volume 5 μl] was incubated in the presence of various concentrations of test substances (0 μM, and 10 measurement points within the range 0.001-20 μM in duplicate) in assay buffer [50 mM HEPES pH 7.0, 1.0 mM MgCl₂, 1.0 mM MgCl₂, 0.1 mM Na ortho-vanadate, 1.0 mM dithiothreitol, 0.001% NP40, 10 μM ATP, 0.03 μM substrate peptide biotin-poly GluTyr, 1.0% dimethyl sulphoxide] at 22° C. for 30 min. The reaction was stopped by adding 5 μl of an EDTA/detection solution [50 mM HEPES pH 7.5, 80 mM EDTA, 0.2% BSA, 0.1 μM streptavidin-XLent (from CisBio), 1 nM PT66-Eu (from PerkinElmer)]. The fluorescence emission at 620 nm and 665 nm after excitation with light of the wavelength 350 nm was measured in a Rubystar HTRF instrument from BMG Labsystems. The measured data (ratio of emission 665 divided by emission 620 multiplied by 10 000) were normalized to 0% inhibition (enzyme reaction without inhibitor) and 100% inhibition (all assay components apart from enzyme). The IC50 values were determined by means of a 4-parameter fit using the company's own software.

Assay 7

KDR Kinase Assay

Recombinant GST-KDR protein was expressed in SF9 insect cells and then purified. The kinase substrate used was the biotinylated peptide biotin-polyGluAlaTyr from Cisbio International.

GST-KDR [test volume 15 μl] was incubated in the presence of various concentrations of test substances (0 μM, and 10 measurement points within the range 0.001-20 μM in duplicate) in assay buffer [50 mM HEPES pH 7.0, 25 mM MgCl₂, 5 mM MgCl₂, 0.5 mM Na ortho-vanadate, 1 mM dithiothreitol, 10% glycerol, 1 μM ATP, 23.5 mg/L substrate peptide biotin-polyGluAlaTyr, 1% dimethyl sulphoxide, 1× protease inhibitor mix (from Roche)] at 22° C. for 20 min. The reaction was stopped by adding 5 μl of an EDTA/detection solution [50 mM HEPES pH 7.0, 250 mM EDTA, 0.5% BSA, 22 mg/L streptavidin-XL (from CisBio), 1 mg/L PT66-Eu (from PerkinElmer)]. The fluorescence emission at 620 nm and 665 nm after excitation with light of the wavelength 350 nm was measured in a Rubystar HTRF instrument from BMG Labsystems 60 minutes after addition of the EDTA/detection solution.

The measured data (ratio of emission 665 divided by emission 620 multiplied by 10 000) were normalized to 0% inhibition (enzyme reaction without inhibitor) and 100% inhibition (all assay components apart from enzyme). The IC50 values were determined by means of a 4-parameter fit using the company's own software.

Assay 8

Tie-2 Kinase Assay

Recombinant Tie-2 protein was expressed in Hi5 insect cells and then purified. The kinase substrate used was the biotinylated peptide having the amino acid sequence biotin-EPKDDAYPLYSDFG, which was purchased from Biosynthan. Tie-2 [concentration in the mixture 5 ng/μl] was preincubated in the presence of 100 μM ATP in assay buffer [50 mM HEPES pH 7.0, 0.5 mM MgCl₂, 1.0 mM dithiothreitol, 0.01% NP40, 1 tablet/2.5 ml complete protease inhibitor (from Roche)] at 22° C. for 20 min. The enzyme reaction [0.5 ng/μl Tie-2 in the test mixture, test volume 5 μl] then took place in the presence of various concentrations of test substances (0 μM, and 10 measurement points within the range 0.001-20 μM in duplicate) in assay buffer with 10 μM ATP, 1.0 μM substrate peptide biotin-EPKDDAYPLYSDFG, 1.0% dimethyl sulphoxide for 20 min. The reaction was stopped by adding 5 μl of an EDTA/detection solution [50 mM HEPES pH 7.5, 89 mM EDTA, 0.28% BSA, 0.2 μM streptavidin-XLent (from CisBio), 2 nM PT66-Eu (from PerkinElmer)]. The fluorescence emission at 620 nm and 665 nm after excitation with light of the wavelength 350 nm was measured in a Rubystar HTRF instrument from BMG Labsystems.

The measured data (ratio of emission 665 divided by emission 620 multiplied by 10 000) were normalized to 0% inhibition (enzyme reaction without inhibitor) and 100% inhibition (all assay components apart from enzyme). The IC50 values were determined by means of a 4-parameter fit using the company's own software.

### EXAMPLE 18

The compounds of Examples 1 to 9 were tested in the various kinase assays for their inhibitory effect.

Table 1 shows that the compounds according to the invention inhibit Aurora in the nanomolar range, whereas the inhibition of CDKs is weaker. The examples further demonstrate that the inhibition profiles can be adjusted by structural alterations. Thus, for example, compounds No. 4, No. 7 and No. 9 represent potent combined Aurora, c-kit and VEGF-R2 (KDR) inhibitors.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding European application No. 06090001.6, filed Jan. 3, 2006, and U.S. Provisional Application Ser. No. 60/835,862, filed Aug. 7, 2006, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

TABLE 1
IC₅₀ values

| | | | | | | c-kit | KDR | Tie-2 |
|---|---|---|---|---|---|---|---|---|
| Example | Aurora-C, | Aurora-A, | CDK1 | CDK2 | Chk1 | kinase | kinase | kinase |
| No. | [nM] | [nM] | [nM] | [nM] | [nM] | [nM] | [nM] | [nM] |
| 1 | 19 | 27 | 173 | 323 | 4643 | 29 | 61 | 2920 |
| 2 | 13 | 26 | 104 | 229 | 3626 | 46 | 198 | |
| 3 | 69 | 77 | 210 | 990 | 6839 | 64 | 289 | |
| 4 | 24 | 31 | 118 | 319 | 1850 | 23 | 9 | |
| 5 | 88 | 844 | >1000 | >1000 | >20 000 | 164 | 22 | |
| 6 | 19 | 21 | 70 | 71 | 3769 | 14 | 22 | 1413 |
| 7 | 34 | 44 | 452 | >1000 | >20 000 | 17 | 7 | 1699 |
| 8 | 283 | | 438 | >1000 | >20 000 | 357 | 136 | 17 110 |
| 9 | 95 | 652 | >1000 | >1000 | >12 500 | >12 500 | 60 | >10 000 |

1. Compounds of the general formula I, 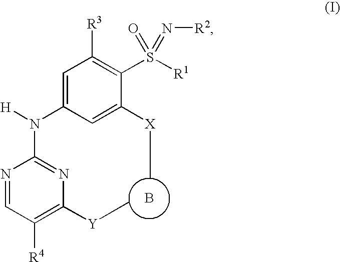
in which
B is a prop-1,3-ylene, but-1,4-ylene, pent-1,5-ylene or hex-1,6-ylene group which may be substituted one or more times, identically or differently, by
(i) hydroxy, —NR¹¹R¹², cyano, halogen, —CF₃, C₁-C₆-alkoxy, —NR¹³—C(O)—C₁-C₃-alkyl, —NR¹³—SO₂—C₁-C₃-alkyl, —OCF₃ and/or
(ii) one or more C₁-C₆-alkyl radicals which are optionally substituted one or more times, identically or differently, by hydroxy, —NR¹¹R¹², cyano, halogen, —CF₃, C₁-C₆-alkoxy, —NR¹³—C(O)—C₁-C₃-alkyl, —NR¹³—SO₂—C₁-C₃-alkyl or —OCF₃,
R¹ is
(i) a C₁-C₆-alkyl radical which is optionally substituted one or more times, identically or differently, by hydroxy, —NR¹¹R¹², cyano, halogen, C₁-C₆-alkoxy, —F₃ and/or —OCF₃, or
(ii) a C₃-C₇-cycloalkyl ring which is optionally substituted one or more times, identically or differently, by hydroxy, —NR¹¹R¹², cyano, halogen, —CF₃, C₁-C₆-alkoxy, —OCF₃ and/or C₁-C₆-alkyl, or
(iii) a C₆-aryl ring which is optionally substituted one or more times, identically or differently, by hydroxy, —NR¹¹R¹², cyano, halogen, —CF₃, C₁-C₆-alkoxy, —OCF₃ and/or C₁-C₆-alkyl, or
(iv) a heteroaryl ring which is optionally substituted one or more times, identically or differently, by hydroxy, —NR¹¹R¹², cyano, halogen, —CF₃, C₁-C₆-alkoxy, —OCF₃ and/or C₁-C₆-alkyl and has 5 or 6 ring atoms,
R² is R⁵, —SO₂—R⁶, —C(O)O—R⁶, —C(O)—R⁶, —C(O)—NR¹¹R¹², —C(S)—NR¹¹R¹², —Si(R⁷R⁸R⁹), —R¹⁰—Si(R⁷R⁸R⁹) or —SO₂—R¹⁰—Si(R⁷R⁸R⁹),
R³ is
(i) hydrogen, hydroxy, halogen, cyano, —CF₃, C₁-C₆-alkoxy —OCF₃ or —NR¹¹R¹², or
(ii) a C₁-C₆-alkyl radical which is optionally substituted one or more times, identically or differently, by halogen, hydroxy, C₁-C₆-alkoxy or the group —NR¹¹R¹², or
(iii) a C₁-C₆-alkoxy group which is optionally substituted one or more times, identically and/or differently, by halogen, hydroxy, C₁-C₆-alkoxy or the group —NR¹¹R¹², or
(iv) a C₃-C₇-cycloalkyl ring which is optionally substituted one or more times, identically or differently, by halogen, hydroxy, C₁-C₆-alkoxy, the group —NR¹¹R¹² and/or C₁-C₆-alkyl,
R⁴ is
(i) halogen, cyano, nitro, —NR¹¹R¹², —CF₃, C₁-C₆-alkoxy or —OCF₃ or
(ii) a C₁-C₆-alkyl, C₂-C₆-alkenyl or C₂-C₆-alkynyl radical which is optionally substituted one or more times, identically or differently, by hydroxy, —NR¹¹R¹², cyano, halogen, C₁-C₆-alkoxy, —CF₃ and/or —OCF₃, or
(iii) a C₆-aryl ring which is optionally substituted one or more times, identically or differently, by hydroxy, —NR¹¹R¹², cyano, halogen, —CF₃, C₁-C₆-alkoxy, —OCF₃ and/or C₁-C₆-alkyl, or
(iv) a heteroaryl ring which is optionally substituted one or more times, identically or differently, by hydroxy, —NR¹¹R¹², cyano, halogen, —CF₃, C₁-C₆-alkoxy, —OCF₃ and/or C₁-C₆-alkyl and has 5 or 6 ring atoms,
X is —S—, —S(O), —NH— or —O—,
Y is —NR¹³—, —S—, —S(O)—, or —O—,
where
R⁵ may be a
(i) C₁-C₆-alkyl radical or
(ii) C₃-C₆-alkenyl radical or
(iii) C₃-C₆-alkynyl radical or
(iv) C₆-aryl ring or
(v) heteroaryl ring having 5 or 6 ring atoms,
each of which may optionally be substituted one or more times, identically or differently, by hydroxy, —NR¹¹R¹², cyano, halogen, —CF₃, C₁-C₆-alkoxy and/or —OCF₃,
R⁶ may be a
(i) C₁-C₆-alkyl radical or
(ii) C₂-C₆-alkenyl radical or
(iii) C₂-C₆-alkynyl radical or
(iv) C₆-aryl ring or
(v) heteroaryl ring having 5 or 6 ring atoms,
each of which may optionally be substituted one or more times, identically or differently, by hydroxy, —NR¹¹R¹², cyano, halogen, —CF₃, C₁-C₆-alkoxy and/or —OCF₃,
R⁷, R⁸
and R⁹ may be independently of one another
(i) a C₁-C₆-alkyl radical, and/or
(ii) a C₆-aryl ring,
R¹⁰ is a C₁-C₃-alkylene group, and
R¹¹ and R¹² may be independently of one another
(i) hydrogen and/or
(ii) a C₁-C₆-alkyl radical, a C₃-C₇-cycloalkyl radical, a C₂-C₆-alkenyl radical, and/or
(iii) a C₆-aryl ring and/or
(iv) a heteroaryl ring having 5 or 6 ring atoms, where (ii), (iii) and (iv) may optionally be substituted one or more times, identically or differently, by hydroxy, —NR¹³R¹⁴, cyano, halogen, —CF₃, C₁-C₆-alkoxy and/or —OCF₃, or
R¹¹ and R¹² together with the nitrogen atom form a heterocyclyl ring which has 3 to 7 ring atoms and may optionally be substituted one or more times, identically or differently, by hydroxy, —NR¹³R¹⁴, cyano, halogen, —CF₃, C₁-C₆-alkoxy and/or —OCF₃ and may comprise a further heteroatom, and
R¹³ and R¹⁴ are independently of one another hydrogen or a C₁-C₆-alkyl radical which is optionally substituted one or more times, identically or differently, by hydroxy, —NH₂, cyano, halogen, —CF₃, C₁-C₆-alkoxy and/or —OCF₃,
and the salts, diastereomers and enantiomers thereof.

2. Compounds according to claim 1,
in which
R¹¹ and R¹² may be independently of one another
(i) hydrogen and/or
(ii) a C₁-C₆-alkyl radical, and/or
(iii) a C₆-aryl ring and/or
(iv) a heteroaryl ring having 5 or 6 ring atoms,
where (ii), (iii) and (iv) may optionally be substituted one or more times, identically or differently, by hydroxy, —NR¹³R¹⁴, cyano, halogen, —CF₃, C₁-C₆-alkoxy and/or —OCF₃,
and the salts, diastereomers and enantiomers thereof.

3. Compounds according to claim 1,
in which
R¹ is a C₁-C₆-alkyl radical which is optionally substituted one or more times, identically or differently, by hydroxy, —NR¹¹R¹², cyano, halogen, —CF₃, C₁-C₆-alkoxy and/or —OCF₃, R² is R⁵, —SO₂—R⁶, —C(O)O—R⁶, —C(O)—R⁶, —C(O)—NR¹¹R¹² or —SO₂—R¹⁰—Si(R⁶R⁷R⁸),
R³ is hydrogen,
R⁴ is
(i) halogen or
(ii) a C₆-aryl ring which is optionally substituted one or more times, identically or differently, by hydroxy, —NR¹¹R¹², cyano, halogen,
—CF₃, C₁-C₆-alkoxy, —OCF₃ and/or C₁-C₆-alkyl, or
(iii) a heteroaryl ring which is optionally substituted one or more times, identically or differently, by hydroxy, —NR¹¹R¹², cyano, halogen,
—CF₃, C₁-C₆-alkoxy, —OCF₃ and/or C₁-C₆-alkyl and has 5 or 6 ring atoms,
X is —O— or —NH—,
Y is —S— or —NH—
where
R⁵, R⁶, R⁷,
R⁸ and R⁹ are independently of one another a C₁-C₆-alkyl radical which are optionally substituted one or more times, identically or differently, by hydroxy, —NR¹¹R¹², cyano, halogen, —CF₃, C₁-C₆-alkoxy and/or —OCF₃,
R¹⁰ is a C₁-C₃-alkylene group,
R¹¹ and R¹² may be independently of one another
(i) hydrogen and/or
(ii) a C₁-C₆-alkyl radical,
where (ii) is optionally substituted one or more times, identically or differently, by hydroxy, —NR¹³R¹⁴, cyano, halogen, —CF₃, C₁-C₆-alkoxy and/or —OCF₃, and
R¹³ and R¹⁴ may be independently of one another hydrogen or a C₁-C₆-alkyl radical which is optionally substituted one or more times, identically or differently, by hydroxy,
—NH₂, cyano, halogen, —CF₃, C₁-C₆-alkoxy and/or —OCF₃,
and B has the meaning stated in claim 1,
and the salts, diastereomers and enantiomers thereof.

4. Compounds according to claim 1,
in which
B is a prop-1,3-ylene, but-1,4-ylene or pent-1,5-ylene group which may optionally be substituted one or more times, identically or differently, by hydroxy and/or one or more C₁-C₆-alkyl radicals which are optionally substituted one or more times, identically or differently, by —NR¹¹R¹², cyano, halogen, —CF₃, C₁-C₆-alkoxy, —NR¹³—C(O)—C₁-C₃-alkyl, —NR¹³—SO₂—C₁-C₃-alkyl or —OCF₃,
and the salts, diastereomers and enantiomers thereof.

5. Compounds according to claim 1,
B is a prop-1,3-ylene, but-1,4-ylene or pent-1,5-ylene group which may optionally be substituted one or more times, identically or differently, by hydroxy and/or one or more C₁-C₆-alkyl radicals which are optionally substituted one or more times, identically or differently, by —NR¹¹R¹², C₁-C₆-alkoxy, —NR¹³—C(O)—C₁-C₃-alkyl or —NR¹³—SO₂—C₁-C₃-alkyl,
and the salts, diastereomers and enantiomers thereof.

6. Compounds according to claim 1,
in which
B is a but-1,4-ylene group which may be substituted one or more times, identically or differently, by hydroxy and/or one or more C₁-C₆-alkyl radicals which are optionally substituted one or more times, identically or differently, by —NR¹¹R¹², C₁-C₆-alkoxy, —NR¹³—C(O)—C₁-C₃-alkyl or —NR¹³—SO₂—C₁-C₃-alkyl,
and the salts, diastereomers and enantiomers thereof.

7. Compounds according to claim 1,
in which
Y is —NH— or —S—,
and the salts, diastereomers and enantiomers thereof.

8. Compounds according to claim 1,
in which
X is —NH— or —O—,
and the salts, diastereomers and enantiomers thereof.

9. Compounds according to claim 1,
in which
R³ is
(i) hydrogen, hydroxy, halogen, C₁-C₆ alkoxy, —NR¹¹R¹², or
(ii) a —C₁-C₆-alkyl radical which is optionally substituted one or more times, identically or differently, by halogen, hydroxy, C₁-C₆-alkoxy or the group —NR¹¹R¹² or
(iii) a C₁-C₆-alkoxy group which is optionally substituted one or more times, identically and/or differently, by halogen, hydroxy, C₁-C₆-alkoxy or the group —NR¹¹R¹².

10. Compounds according to claim 1,
in which
R³ is hydrogen,
and the salts, diastereomers and enantiomers thereof.

11. Compounds according to claim 1,
in which
R⁴ is
(i) halogen or —CF₃ or
(ii) C₆-aryl ring which is optionally substituted one or more times, identically or differently, by hydroxy, —NR¹¹R¹², cyano, halogen, —CF₃, C₁-C₆-alkoxy, —OCF₃ and/or C₁-C₆-alkyl, or
(iii) a heteroaryl ring which is optionally substituted one or more times, identically or differently, by hydroxy, —NR¹¹R¹², cyano, halogen, —CF₃, C₁-C₆-alkoxy, —OCF₃ and/or C₁-C₆-alkyl and has 5 or 6 ring atoms, where
R¹¹ and R¹² may be independently of one another
(i) hydrogen and/or
(ii) a C₁-C₆-alkyl radical,
where (ii) is optionally substituted one or more times, identically or differently, by hydroxy, —NR¹³R¹⁴, cyano, halogen, —CF₃, C₁-C₆-alkoxy and/or
—OCF₃, and
R¹³ and R¹⁴ are independently of one another hydrogen or a C₁-C₆-alkyl radical which is optionally substituted one or more times, identically or differently, by hydroxy, —NH₂, cyano, halogen, —CF₃, C₁-C₆-alkoxy and/or —OCF₃.

12. Compounds according to claim 1,
in which
R⁴ is halogen,
and the salts, diastereomers and enantiomers thereof.

13. Compounds according to claim 1,
in which
R² is R⁵, —SO₂—R⁶, —C(O)O—R⁶, —C(O)—R⁶, —C(O)—NR¹¹R¹² or —SO₂—R¹⁰—Si(R⁷R⁸R⁹),
where
R⁵, R⁶, R⁷,
R⁸ and R⁹ is independently of one another a C₁-C₆-alkyl radical which is optionally substituted one or more times, identically or differently, by hydroxy, —NR¹¹R¹², cyano, halogen,
—CF₃, C₁-C₆-alkoxy and/or —OCF₃,
R¹⁰ is a C₁-C₃-alkylene group, and
R¹¹ and R¹² may be independently of one another
(i) hydrogen and/or
(ii) a C₁-C₆-alkyl radical,
where (ii) is optionally substituted one or more times, identically or differently, by hydroxy, —NR¹³R¹⁴, cyano, halogen, —CF₃, C₁-C₆-alkoxy and/or
—OCF₃, and
R¹³ and R¹⁴ may be independently of one another hydrogen or a C₁-C₆-alkyl radical which is optionally substituted one or more times, identically or differently, by hydroxy,
—NH₂, cyano, halogen, —CF₃ and/or —OCF₃.

14. Compounds according to claim 1,
in which
R² is —SO₂—R⁶, —C(O)O—R⁶, —C(O)—NR¹¹R¹² or —SO₂—R¹⁰—Si(R⁷R⁸R⁹),
where
R⁶, R⁷, R⁸ and R⁹ are independently of one another C₁-C₅-alkyl radicals,
R¹⁰ is a C₁-C₅-alkylene group, and
R¹¹ and R¹² may be independently of one another hydrogen and/or a C₁-C₆-alkyl radical,
and the salts, diastereomers and enantiomers thereof.

15. Compounds according to claim 1,
in which
R² is —SO₂—R⁶, —C(O)O—R⁶, —C(O)—R⁶, —C(O)—NR¹¹R¹² or —SO₂—R¹⁰—Si(R⁷R⁸R⁹), where
R⁶ is a C₁-C₆-alkyl radical
R⁷, R⁸
and R⁹ may be independently of one another a C₁-C₆-alkyl radical,
R¹⁰ is a C₁-C₃-alkylene group,
R¹¹ and R¹² may be independently of one another
(i) hydrogen and/or
(ii) a C₁-C₆-alkyl radical, a C₃-C₇-cycloalkyl radical, a C₂-C₆-alkenyl radical, and/or
(iii) a C₆-aryl ring and/or
(iv) a heteroaryl ring having 5 or 6 ring atoms, where (ii), (iii) and (iv) may optionally be substituted one or more times, identically or differently, by hydroxy, —NR¹³R¹⁴, cyano, halogen, —CF₃, C₁-C₆-alkoxy and/or —OCF₃,
R¹³ and R¹⁴ are independently of one another a C₁-C₆-alkyl radical,
and the salts, diastereomers and enantiomers thereof.

16. Compounds according to claim 1,
in which
R⁵, R⁶, R⁷, R⁸ and R⁹ are independently of one another C₁-C₆-alkyl radicals,
and the salts, diastereomers and enantiomers thereof.

17. Compounds according to claim 1,
in which
R¹⁰ is ethylene,
and the salts, diastereomers and enantiomers thereof.

18. Compounds according to claim 1,
in which
R¹¹ and R¹² may be independently of one another
(i) hydrogen and/or
(ii) a C₁-C₆-alkyl radical, a C₃-C₇-cycloalkyl radical, a C₂-C₆-alkenyl radical, and/or
(iii) a C₆-aryl ring and/or
(iv) a heteroaryl ring having 5 or 6 ring atoms, where (ii), (iii) and (iv) may optionally be substituted one or more times, identically or differently, by hydroxy, —NR¹³R¹⁴, cyano, halogen, —CF₃, C₁-C₆-alkoxy and/or —OCF₃, and R¹³ and R¹⁴ are independently of one another C₁-C₆-alkyl radicals
and the salts, diastereomers and enantiomers thereof.

19. Compounds according to claim 1,
in which
R¹¹ and R¹² may be independently of one another hydrogen and/or C₁-C₆-alkyl radicals,
and the salts, diastereomers and enantiomers thereof.

20. Compounds of the general formula I according to claim 1,
in which
B is a but-1,4-ylene group,
R¹ is a C₁-C₆-alkyl radical,
R² is —SO₂—R⁶, —C(O)O—R⁶, —C(O)—R⁶, —C(O)—NR¹¹R¹² or —SO₂—R¹⁰—Si(R⁷R⁸R⁹),
R³ is hydrogen,
R⁴ is halogen or a heteroaryl ring having 5 or 6 ring atoms,
X is —NH— or —O—,
Y is —NR¹³—,
where
R⁶ is a C₁-C₆-alkyl radical,
R⁷, R⁸
and R⁹ may independently of one another be a C₁-C₆-alkyl radical,
R¹⁰ is a C₁-C₃-alkylene group,
R¹¹ and R¹² may be independently of one another
(i) hydrogen and/or
(ii) a C₁-C₆-alkyl radical, a C₃-C₇-cycloalkyl radical, a C₂-C₆-alkenyl radical and/or
(iii) a C₆-aryl ring and/or
(iv) a heteroaryl ring having 5 or 6 ring atoms, where (ii), (iii) and (iv) may optionally be substituted one or more times, identically or differently, by hydroxy, —NR¹³R¹⁴, cyano, halogen, —CF₃, C₁-C₆-alkoxy and/or —OCF₃,
R¹³ and R¹⁴ are independently of one another hydrogen and/or a C₁-C₆-alkyl radical,
and the salts, diastereomers and enantiomers thereof.

21. Compounds of the general formula I according to claim 1,
in which
B is a prop-1,3-ylene, but-1,4-ylene, pent-1,5-ylene or hex-1,6-ylene group,
R¹ is a C₁-C₅-alkyl radical,
R² is —SO₂—R⁵, —C(O)O—R⁶, —C(O)—NR¹¹R¹² or —SO₂—R¹⁰—Si(R⁷R⁸R⁹), where
R⁵, R⁶, R⁷,
R⁸ and R⁹ are independently of one another C₁-C₅-alkyl radicals,
R¹⁰ is a C₁-C₅-alkylene group,
R¹¹ and R¹² may be independently of one another hydrogen and/or C₁-C₆-alkyl radicals,
R³ is hydrogen, and
R⁴ is a halogen,
and the salts, diastereomers and enantiomers thereof.

22. Compounds of the general formula I according to claim 1,
in which
B is a but-1,4-ylene group,
R¹ is a methyl group,
R² is an —SO₂—R⁶, —C(O)O—R⁶, —C(O)—NHR⁶ or —SO₂—C₂H₄—Si(CH₃)₃, where R⁶ can be an ethyl or propyl radical,
R³ is hydrogen,
R⁴ is a halogen,
X is —O— or —NH—, and
Y is —NH—,
and the salts, diastereomers and enantiomers thereof.

23. Compounds of the general formula I according to claim 1 for use as medicaments.

24. Use of compounds of the general formula I according to claim 1 for manufacturing a medicament for the treatment of cancer.

25. Use of compounds of the general formula I according to claim 1 for manufacturing a medicament for the treatment of cardiovascular disorders.

26. Process for preparing a compound according to claim 1, characterized by the steps:
a) functionalization of position 4 of 2,4-dichloropyrimidine derivatives of the formula 1a by reaction with nucleophiles under basic conditions, where appropriate with use of a protective group for group X, which is eliminated again where appropriate after introduction of 1b into position 4 of 1a, 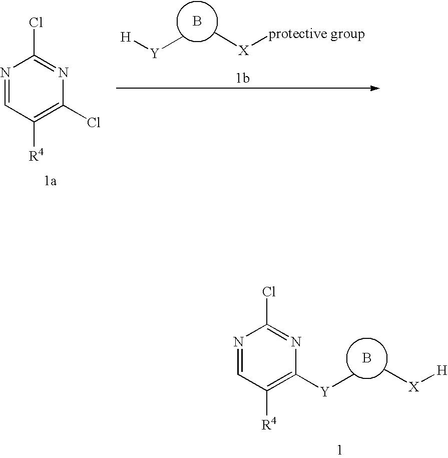
b) oxidation of a compound of the formula 2a to the sulphoxide of the formula 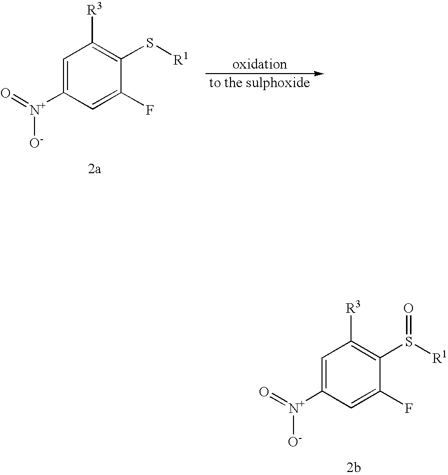
c₁) reaction of the compound of the formula 2b with sodium azide/sulphuric acid to give a compound of the formula 2c and N-functionalization of the sulphoximine to give a compound of the formula 2 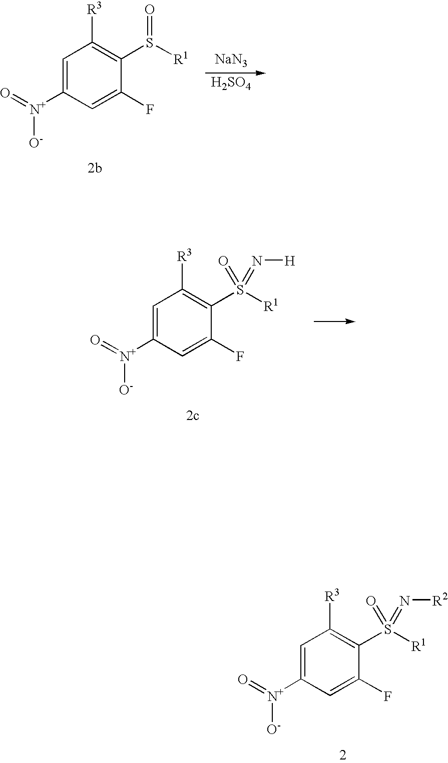
or
c₂) direct reaction of the sulphoxide of the formula 2b to give a compound of the formula 2, 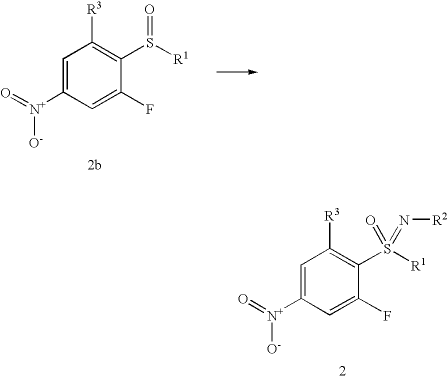
d) reaction of the compound of the formula 1 from process step a) with the compound of the formula 2 from process step b) by a nucleophilic aromatic substitution to give a compound of the formula 3 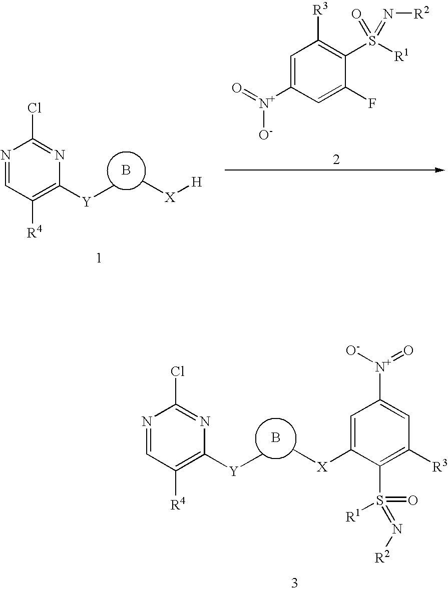
e) reduction of the compound of the formula 3 to a compound of the formula 4 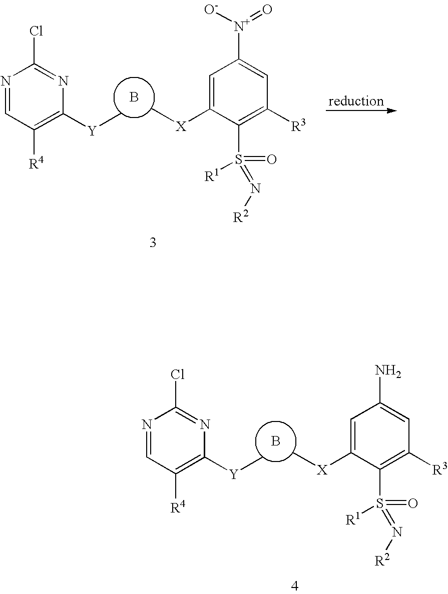
f) cyclization of the compound of the formula 4 under acidic or neutral conditions to give compounds of the formula I

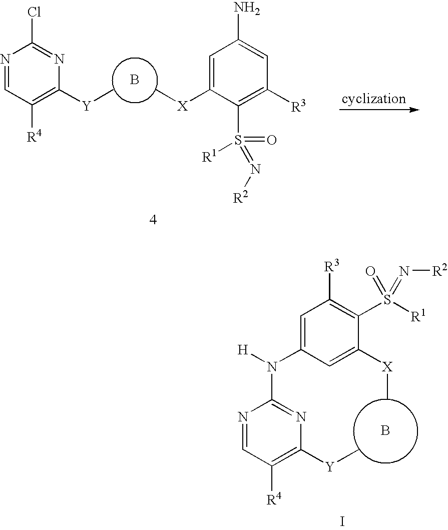

27. Process for preparing a compound according to claim 1, in which X is —O—, characterized by the steps:
a) reaction of an alcohol of the formula 6 with a phenol of the formula 7 under Mitsunobu conditions 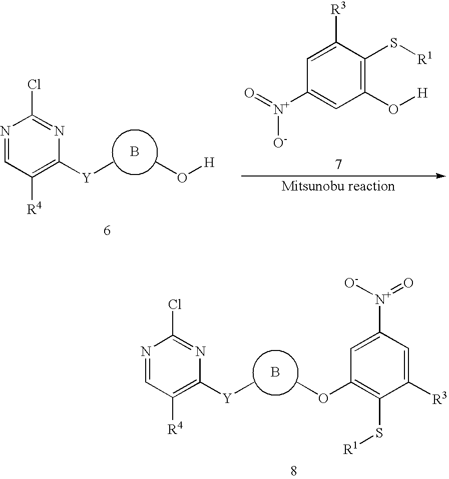
b₁) (i) oxidation of the thioether of the formula 8 to the sulphoxide and subsequently (ii) reaction to give the sulphoximine of the formula 9. 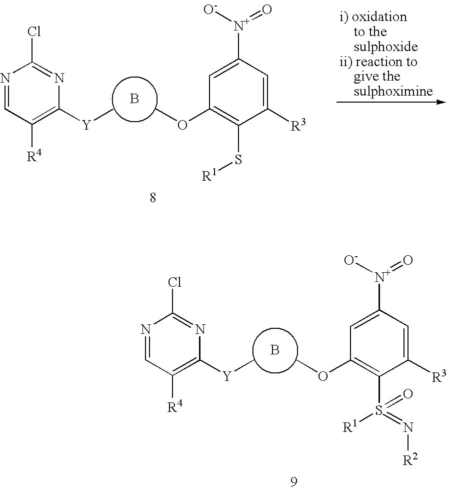
b₂) optionally in the case of compounds of the type 9, in which R2=H, an N-functionalization of the sulphoximine can take place. 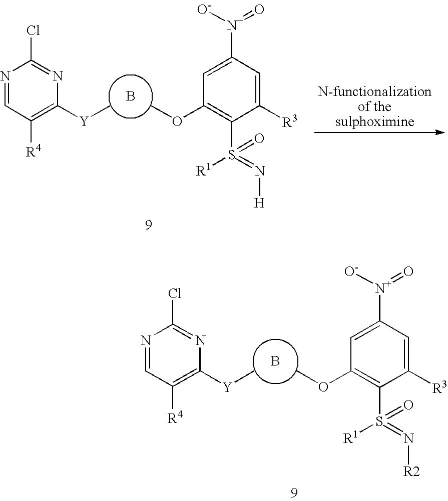
c₁) (i) reduction of the compound of the formula 9 and
(ii) cyclization under acidic or neutral conditions to give compounds of the formula II. 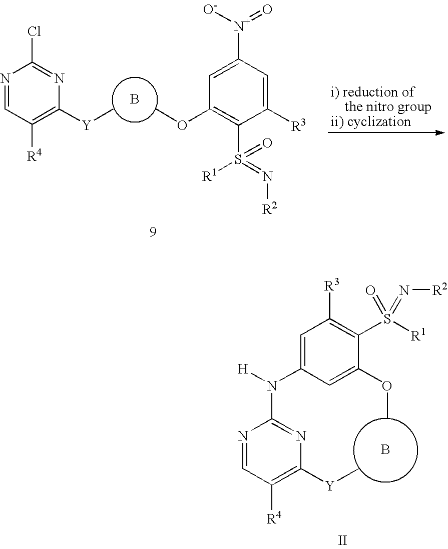
c₂) optionally in the case of compounds of the formula II, in which R2=H, an N-functionalization of the sulphoximine can take place.

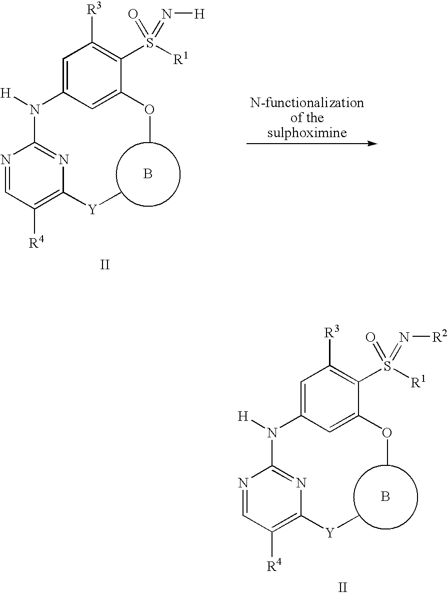

28. Pharmaceutical formulation comprising one or more compounds according to claim 1.
